(19) United States
(12) Patent Application Publication
(10) Pub. No.: US 2025/0255931 A1
(43) Pub. Date: Aug. 14, 2025

(54) METHODS FOR TREATING LEUKOCYTOSIS, ENDOTHELIAL DYSFUNCTION AND CARDITIS USING LIPID BINDING PROTEIN-BASED COMPLEXES

(71) Applicant: ABIONYX PHARMA SA, Balma (FR)

(72) Inventors: Cyrille TUPIN, Castelmaurou (FR); Constance PEYROTTES, Le Castéra (FR); Ronald BARBARAS, Seilh (FR)

(73) Assignee: ABIONYX PHARMA SA, Balma (FR)

(21) Appl. No.: 18/854,185

(22) PCT Filed: Apr. 6, 2023
(86) PCT No.: PCT/IB2023/000183

§ 371 Date: Oct. 4, 2024

Related U.S. Application Data
(60) Provisional application No. 63/328,210, filed on Apr. 6, 2022.

(51) Int. Cl. A61K 38/17; A61K 47/54; A61P 9/00; A61P 9/10; A61P 13/12; A61P 31/14

(52) U.S. Cl. CPC A61K 38/1709; A61K 47/544; A61P 9/00; A61P 9/10; A61P 13/12; A61P 31/14

(57) ABSTRACT

Methods for treating leukocytosis, endothelial dysfunction, and carditis comprising administering to a subject a lipid binding protein-based complex.

### 1. CROSS-REFERENCE TO RELATED APPLICATIONS

This application claims priority to U.S. provisional application 63/328,210, filed Apr. 6, 2022, the contents of which are incorporated herein in their entireties by reference thereto.

### 2. SEQUENCE LISTING

The instant application contains a Sequence Listing which has been submitted electronically and is hereby incorporated by reference in its entirety. Said electronic copy, created on Apr. 3, 2023, is an XML file named CRN-048WO_ST26 and is 3,244 bytes in size.

### 3. SUMMARY

This disclosure is based, in part, on the discovery that subjects treated with the lipid binding protein-based complex CER-001 as described in Example 2 showed an unexpectedly rapid improvement in biomarkers of inflammation, leukocytosis, and endothelial dysfunction.

Accordingly, in some aspects, the disclosure provides methods for treating subjects having or at risk of developing leukocytosis with a dose (e.g., a high dose) of a lipid binding protein-based complex.

In some aspects, the disclosure provides methods for treating subjects having one or more symptoms associated with leukocytosis, comprising administering to the subject a dose of a lipid binding protein-based complex with a dose (e.g., a high dose) of a lipid binding protein-based complex.

In some aspects, the disclosure provides methods for treating subjects having endothelial dysfunction (e.g., subjects having acute coronary syndrome or stroke or who has experienced acute coronary syndrome or stroke) with a dose (e.g., a high dose) of a lipid binding protein-based complex.

In some aspects, the disclosure provides methods of treating a subject experiencing acute coronary syndrome or stroke or who has experienced acute coronary syndrome or stroke with a high dose of a lipid binding protein-based complex.

In some aspects, the disclosure provides methods of treating a subject having or at risk of developing carditis with a dose (e.g., a high dose) of a lipid binding protein-based complex.

In the methods of the disclosure, the lipid binding-protein based complex is typically administered in a high dose. A high dose is typically higher than a dose that would be used to treat a chronic condition such as familial hypercholesterolemia. The high dose is typically administered over a relatively short period of time, for example, over a period of two days to two weeks, and typically comprises multiple administrations of the lipid binding protein-based complex, for example two to 20 individual doses. The individual doses can be separated by less than one day (e.g., twice daily administration), or one day or more (e.g., once daily administration).

In some embodiments of the methods of the disclosure, the lipid binding protein-based complex comprises a sphingomyelin and/or a negatively charged lipid, for example CER-001. CER-001 is a negatively charged lipoprotein complex, and comprises recombinant human ApoA-I, sphingomyelin (SM), and 1, 2-dihexadecanoyl-sn-glycero-3-phospho-(1′-rac-glycerol) (Dipalmitoylphosphatidyl-glycerol; DPPG). It mimics natural, nascent discoidal pre-beta HDL, which is the form that HDL particles take prior to acquiring cholesterol. Without being bound by theory, it is believed that CER-001 therapy can (1) reduce serum levels of inflammatory cytokines such as IL-6, (2) reduce white blood cell count in subject having leukocytosis or at risk of leukocytosis, and (3) reduce serum levels of ICAM-1 and VCAM-1, thereby providing a clinical benefit to subjects having or at risk of developing leukocytosis, subjects having endothelial dysfunction, and subjects having or at risk of developing carditis.

In some aspects, the present disclosure provides dosing regimens for lipid binding protein-based therapy (e.g., CER-001 therapy) for subjects as described herein.

The dosing regimens of the disclosure typically entail multiple administrations of CER-001 to a subject (e.g., administered daily or administered approximately 12 hours apart). The CER-001 therapy can be continued for a pre-determined period, e.g., for one week or a period longer than one week (e.g., two weeks). Alternatively, administration of CER-001 to a subject can be continued until one or more symptoms of a condition experienced by the subject are reduced or continued until the serum levels of one or more inflammatory markers are reduced, for example reduced to a normal level or reduced relative to a baseline measurement taken prior to the start of CER-001 therapy. For subjects having an infection, the therapy can in some embodiments be continued until the subject has recovered from the infection.

The dosing regimens of the disclosure can entail administering a lipid binding protein-based complex (e.g., CER-001) to a subject according to an initial “induction” regimen, optionally followed by administering the lipid binding protein-based complex to the subject according to a “consolidation” regimen.

The induction regimen typically comprises administering multiple doses of the lipid binding protein-based complex (e.g., CER-001) to the subject, for example six doses over three days.

The consolidation regimen typically comprises administering one or more doses of a lipid binding protein-based complex (e.g., CER-001) to the subject following the final dose of the induction regimen, for example one or more days after the final dose of the induction regimen. In some embodiments, the first dose of the consolidation regimen is administered on the third day after the final dose of the induction regimen. For example, a dosing regimen can comprise administration of a lipid binding protein-based complex (e.g., CER-001) to a subject according to an induction regimen on days 1, 2, and 3, and administration of the lipid binding protein-based complex to the subject according to a consolidation regimen on day 6. In some embodiments, the consolidation regimen comprises two doses of the lipid binding protein-based complex.

In certain embodiments, the disclosure provides methods of treating a subject as described herein with a lipid binding protein-based complex (e.g., CER-001) according to a dosage regimen comprising:
2 doses per day on days 1, 2, and 3 (induction regimen) optionally followed by
2 subsequent doses on day 4 or later (consolidation regimen).
   In some embodiments, the regimen comprises:
2 doses per day on days 1, 2, and 3 (induction regimen) followed by
2 doses on day 6 (consolidation regimen).

In some embodiments, the dosage regimen comprises a single phase, e.g., corresponding to an induction regimen described herein.

In certain aspects, a lipid binding protein-based complex (e.g., CER-001) is administered in combination with a standard of care therapy such as antibiotic therapy for an infection and/or hemodynamic support.

In certain aspects, an antihistamine (e.g., dexchlorpheniramine, hydroxyzine, diphenhydramine, cetirizine, fexofenadine, or loratadine) can be administered before administration of a lipid binding protein-based complex (e.g., CER-001). The antihistamine can reduce the likelihood of allergic reactions.

### 4. BRIEF DESCRIPTION OF THE FIGURES

FIG. 1: shows IL-6 serum levels in a pig model of sepsis-induced AKI (Example 1).

FIG. 2: shows soluble VCAM-1 serum levels in a pig model of sepsis-induced AKI (Example 2).

FIG. 3: shows soluble ICAM-1 serum levels in a pig model of sepsis-induced AKI (Example 3).

FIG. 4: shows LPS serum levels in a pig model of sepsis-induced AKI (Example 1).

FIG. 5: shows a schematic of the clinical study of Example 2.

FIG. 6 is a flowsheet for the study of Example 3.

FIG. 7 is a flowsheet for the study of Example 4.

FIGS. 8A-8J show change in MCP1 (FIG. 8A), TNF-α (FIG. 8B), VCAM (FIG. 8C), ICAM (FIG. 8D), Ferritin (FIG. 8E), white blood cell (FIG. 8F), CRP (FIG. 8G), KIM-1 (FIG. 8H), IL-8 (FIG. 8I), and triglycerides (FIG. 8J) in the first cohort of subjects treated in the study of Example 2.

FIGS. 9A-9F show VCAM changes for the standard of care (SOC) group and the three CER-001 groups in the clinical study of Example 2. FIG. 9A, changes from baseline for SOC group versus aggregated CER-001 groups. FIG. 9B, changes from baseline for each of group. FIG. 9C, changes as a percentage of peak for the SOC group and the aggregated study groups. FIG. 9D, changes as a percentage of peak for the SOC group and the study groups, broken out by whether the subject was enrolled from the ICU or the nephrology department of the center. FIG. 9E, changes from baseline for each subject in the SOC and the aggregated CER-001 groups. FIG. 9F changes from baseline for each subject in the SOC group and each study group.

FIGS. 10A-10F show ICAM changes for the standard of care (SOC) group and the three CER-001 groups in the clinical study of Example 2. FIG. 10A, changes from baseline for SOC group versus aggregated CER-001 groups. FIG. 10B, changes from baseline for each of group. FIG. 10C, changes as a percentage of peak for the SOC group and the aggregated study groups. FIG. 10D, changes as a percentage of peak for the SOC group and the study groups, broken out by whether the subject was enrolled from the ICU or the nephrology department of the center. FIG. 10E, changes from baseline for each subject in the SOC and the aggregated CER-001 groups. FIG. 10F changes from baseline for each subject in the SOC group and each study group.

FIG. 11 shows MTT cell viability assay results for cultured endothelial cells upon challenge with LPS and CER-001 infusions, as described in Example 6.

FIG. 12 summarizes endothelial nitric oxide synthase (eNOS)-based (eNOS(phosphoS1177)) FACS results for cultured endothelial cells upon challenge with LPS and CER-001 infusions, as described in Example 6.

FIG. 13 shows eNOS(phosphoS1177)-based FACS results for cultured endothelial cells upon challenge with LPS and CER-001 infusions in one representative of three independent experiments, compared to basal and VEFG (positive control) cells, as described in Example 6.

FIG. 14 shows MTT cell viability assay results for PBMCs from healthy donors upon challenge with LPS and CER-001 infusions, as described in Example 6.

FIG. 15 shows TNF-α synthesis for PBMCs from healthy donors upon challenge with LPS and CER-001 infusions, as described in Example 6.

FIG. 16 shows CD14-based FACS results for PBMCs from healthy donors upon challenge with LPS and CER-001 infusions in one representative of three independent experiments, as described in Example 6.

FIG. 17 summarizes CD14-based FACS results for PBMCs from healthy donors upon challenge with LPS and CER-001 infusions, as described in Example 6.

FIG. 18A-FIG. 18F show white blood cell count changes for the standard of care (SOC) group and the CER-001 groups in the clinical study of Example 2. FIG. 18A: white blood cell count changes from baseline for SOC group and aggregated CER-001 groups. FIG. 18B: white blood cell count changes from baseline for SOC group and each CER-001 group. FIG. 18C: white blood cell count changes, reported as a percentage relative to peak white blood cell counts (peak=100%), for SOC group and aggregated CER-001 groups. The treatment x study day effect relative to peak was p=0.5492. FIG. 18D: white blood cell count changes for SOC group and aggregated CER-001 groups, broken out by whether the subject was enrolled from the ICU or the nephrology department of the center. FIG. 18E: individual data points summarized in FIG. 18A. FIG. 18F: individual data points summarized in FIG. 18B.

### 5. DETAILED DESCRIPTION

In some aspects, the disclosure provides methods for treating subjects having or at risk of developing leukocytosis with a dose (e.g., a high dose) of a lipid binding protein-based complex.

In some aspects, the disclosure provides methods for treating subjects having one or more symptoms associated with leukocytosis, comprising administering to the subject a dose of a lipid binding protein-based complex with a dose (e.g., a high dose) of a lipid binding protein-based complex.

In some aspects, the disclosure provides methods for treating subjects having endothelial dysfunction (e.g., subjects having acute coronary syndrome or stroke or who has experienced acute coronary syndrome or stroke) with a dose (e.g., a high dose) of a lipid binding protein-based complex.

In some aspects, the disclosure provides methods of treating a subject experiencing acute coronary syndrome or stroke or who has experienced acute coronary syndrome or stroke with a high dose of a lipid binding protein-based complex.

In some aspects, the disclosure provides methods of treating a subject having or at risk of developing carditis with a dose (e.g., a high dose) of a lipid binding protein-based complex.

In some embodiments of the methods of the disclosure, the lipid binding protein-based complex is an Apomer, a Cargomer, a HDL based complex, or a HDL mimetic based complex. In specific embodiments, the lipid binding protein-based complex is CER-001.

Exemplary features of lipid binding protein-based complexes that can be used in the methods and compositions of the disclosure are described in Section 5.1. Exemplary subject populations who can be treated by the methods of the disclosure and with the compositions of the disclosure are described in Section 5.2.

In some embodiments, methods of the disclosure comprise administering a lipid binding protein-based complex (e.g., CER-001) to a subject in two phases. First, the lipid binding protein-based complex (e.g., CER-001) is administered in an initial, intense “induction” regimen. The induction regimen is followed by a less intense “consolidation” regimen. Alternatively, a lipid binding protein-based complex (e.g., CER-001) can be administered to a subject in a single phase, for example according to an administration regimen corresponding to the dose and administration frequency of an induction or consolidation regimen described herein.

Induction regimens that can be used in the methods of the disclosure are described in Section 5.3 and consolidation regimens that can be used in the methods of the disclosure are described in Section 5.3.2. The dosing regimens of the disclosure comprise administering a lipid binding protein-based complex (e.g., CER-001) as monotherapy or as part of a combination therapy with one or more medications, for example in combination with a standard of care therapy for sepsis or other infection such as antibiotic treatment and/or hemodynamic support. Combination therapies are described in Section 5.4.

### 5.1. Lipid Binding Protein-Based Complexes

### 5.1.1. HDL and HDL Mimetic-Based Complexes

In one aspect, the lipid binding protein-based complexes comprise HDL or HDL mimetic-based complexes. For example, complexes can comprise a lipoprotein complex as described in U.S. Pat. No. 8,206,750, PCT publication WO 2012/109162, PCT publication WO 2015/173633 A2 (e.g., CER-001) or US 2004/0229794 A1, the contents of each of which are incorporated herein by reference in their entireties. The terms “lipoproteins” and “apolipoproteins” are used interchangeably herein, and unless required otherwise by context, the term “lipoprotein” encompasses lipoprotein mimetics. The terms “lipid binding protein” and “lipid binding polypeptide” are also used interchangeably herein, and unless required otherwise by context, the terms do not connote an amino acid sequence of particular length.

Lipoprotein complexes can comprise a protein fraction (e.g., an apolipoprotein fraction) and a lipid fraction (e.g., a phospholipid fraction). The protein fraction includes one or more lipid-binding protein molecules, such as apolipoproteins, peptides, or apolipoprotein peptide analogs or mimetics, for example one or more lipid binding protein molecules described in Section 5.1.2.

The lipid fraction typically includes one or more phospholipids which can be neutral, negatively charged, positively charged, or a combination thereof. Exemplary phospholipids and other amphipathic molecules which can be included in the lipid fraction are described in Section 5.1.4.

In certain embodiments, the lipid fraction contains at least one neutral phospholipid (e.g., a sphingomyelin (SM)) and, optionally, one or more negatively charged phospholipids. In lipoprotein complexes that include both neutral and negatively charged phospholipids, the neutral and negatively charged phospholipids can have fatty acid chains with the same or different number of carbons and the same or different degree of saturation. In some instances, the neutral and negatively charged phospholipids will have the same acyl tail, for example a C16:0, or palmitoyl, acyl chain. In specific embodiments, particularly those in which egg SM is used as the neutral lipid, the weight ratio of the apolipoprotein fraction:lipid fraction ranges from about 1:2.7 to about 1:3 (e.g., 1:2.7).

Any phospholipid that bears at least a partial negative charge at physiological pH can be used as the negatively charged phospholipid. Non-limiting examples include negatively charged forms, e.g., salts, of phosphatidylinositol, a phosphatidylserine, a phosphatidylglycerol and a phosphatidic acid. In a specific embodiment, the negatively charged phospholipid is 1,2-dipalmitoyl-sn-glycero-3-[phospho-rac-(1-glycerol)], or DPPG, a phosphatidylglycerol. Preferred salts include potassium and sodium salts.

In some embodiments, a lipoprotein complex used in the methods of the disclosure is a lipoprotein complex as described in U.S. Pat. No. 8,206,750 or WO 2012/109162 (and its U.S. counterpart, US 2012/0232005), the contents of each of which are incorporated herein in its entirety by reference. In particular embodiments, the protein component of the lipoprotein complex is as described in Section 6.1 and preferably in Section 6.1.1 of WO 2012/109162 (and US 2012/0232005), the lipid component is as described in Section 6.2 of WO 2012/109162 (and US 2012/0232005), which can optionally be complexed together in the amounts described in Section 6.3 of WO 2012/109162 (and US 2012/0232005). The contents of each of these sections are incorporated by reference herein. In certain aspects, a lipoprotein complex of the disclosure is in a population of complexes that is at least 85%, at least 90%, at least 95%, at least 97%, or at least 99% homogeneous, as described in Section 6.4 of WO 2012/109162 (and US 2012/0232005), the contents of which are incorporated by reference herein.

In a specific embodiment, a lipoprotein complex that can be used in the methods of the disclosure comprises 2-4 ApoA-I equivalents, 2 molecules of charged phospholipid, 50-80 molecules of lecithin and 20-50 molecules of SM.

In another specific embodiment, a lipoprotein complex that can be used in the methods of the disclosure comprises 2-4 ApoA-I equivalents, 2 molecules of charged phospholipid, 50 molecules of lecithin and 50 molecules of SM.

In yet another specific embodiment, a lipoprotein complex that can be used in the methods of the disclosure comprises 2-4 ApoA-I equivalents, 2 molecules of charged phospholipid, 80 molecules of lecithin and 20 molecules of SM.

In yet another specific embodiment, a lipoprotein complex that can be used in the methods of the disclosure comprises 2-4 ApoA-I equivalents, 2 molecules of charged phospholipid, 70 molecules of lecithin and 30 molecules of SM.

In yet another specific embodiment, a lipoprotein complex that can be used in the methods of the disclosure comprises 2-4 ApoA-I equivalents, 2 molecules of charged phospholipid, 60 molecules of lecithin and 40 molecules of SM.

In a specific embodiment, a lipoprotein complex that can be used in the methods of the disclosure consists essentially of 2-4 ApoA-I equivalents, 2 molecules of charged phospholipid, 50-80 molecules of lecithin and 20-50 molecules of SM.

In another specific embodiment, a lipoprotein complex that can be used in the methods of the disclosure consists essentially of 2-4 ApoA-I equivalents, 2 molecules of charged phospholipid, 50 molecules of lecithin and 50 molecules of SM.

In yet another specific embodiment, a lipoprotein complex that can be used in the methods of the disclosure consists essentially of 2-4 ApoA-I equivalents, 2 molecules of charged phospholipid, 80 molecules of lecithin and 20 molecules of SM.

In yet another specific embodiment, a lipoprotein complex that can be used in the methods of the disclosure consists essentially of 2-4 ApoA-I equivalents, 2 molecules of charged phospholipid, 70 molecules of lecithin and 30 molecules of SM.

In yet another specific embodiment, a lipoprotein complex that can be used in the methods of the disclosure consists essentially of 2-4 ApoA-I equivalents, 2 molecules of charged phospholipid, 60 molecules of lecithin and 40 molecules of SM.

In a specific embodiment, a lipoprotein complex that can be used in the methods of the disclosure comprises a lipid component that comprises about 90 to 99.8 wt % SM and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, or 0.2-10 wt % total negatively charged phospholipid(s). In another specific embodiment, a lipoprotein complex that can be used in the methods of the disclosure comprises about 90 to 99.8 wt % lecithin and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt % or 0.2-10 wt % total negatively charged phospholipid(s).

In a specific embodiment, a lipoprotein complex that can be used in the methods of the disclosure comprises a lipid component that consists essentially of about 90 to 99.8 wt % SM and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, or 0.2-10 wt % total negatively charged phospholipid(s). In another specific embodiment, a lipoprotein complex that can be used in the methods of the disclosure consists essentially of about 90 to 99.8 wt % lecithin and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt % or 0.2-10 wt % total negatively charged phospholipid(s).

In still another specific embodiment, a lipoprotein complex that can be used in the methods of the disclosure comprises a lipid fraction that comprises about 9.8 to 90 wt % SM, about 9.8 to 90 wt % lecithin and about 0.2-10 wt % negatively charged phospholipid, for example, from about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, to 0.2-10 wt % total negatively charged phospholipid(s).

In still another specific embodiment, a lipoprotein complex that can be used in the methods of the disclosure comprises a lipid fraction that consists essentially of about 9.8 to 90 wt % SM, about 9.8 to 90 wt % lecithin and about 0.2-10 wt % negatively charged phospholipid, for example, from about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, to 0.2-10 wt % total negatively charged phospholipid(s).

In another specific embodiment, a lipoprotein complex that can be used in the methods of the disclosure comprises an ApoA-I apolipoprotein and a lipid fraction, wherein the lipid fraction comprises sphingomyelin and about 3 wt % of a negatively charged phospholipid, wherein the molar ratio of the lipid fraction to the ApoA-I apolipoprotein is about 2:1 to 200:1, and wherein said complex is a small or large discoidal particle containing 2-4 ApoA-I

### EQUIVALENTS

In another specific embodiment, a lipoprotein complex that can be used in the methods of the disclosure comprises an ApoA-I apolipoprotein and a lipid fraction, wherein the lipid fraction consists essentially of sphingomyelin and about 3 wt % of a negatively charged phospholipid, wherein the molar ratio of the lipid fraction to the ApoA-I apolipoprotein is about 2:1 to 200:1, and wherein said complex is a small or large discoidal particle containing 2-4 ApoA-I equivalents.

HDL-based or HDL mimetic-based complexes can include a single type of lipid-binding protein, or mixtures of two or more different lipid-binding proteins, which may be derived from the same or different species. Although not required, the complexes will preferably comprise lipid-binding proteins that are derived from, or correspond in amino acid sequence to, the animal species being treated, in order to avoid inducing an immune response to the therapy. Thus, for treatment of human patients, lipid-binding proteins of human origin are preferably used. The use of peptide mimetic apolipoproteins may also reduce or avoid an immune response.

In some embodiments, the lipid component includes two types of phospholipids: a sphingomyelin (SM) and a negatively charged phospholipid. Exemplary SMs and negatively charged lipids are described in Section 5.1.4.1.

Lipid components including SM can optionally include small quantities of additional lipids. Virtually any type of lipids may be used, including, but not limited to, lysophospholipids, galactocerebroside, gangliosides, cerebrosides, glycerides, triglycerides, and cholesterol and its derivatives.

When included, such optional lipids will typically comprise less than about 15 wt % of the lipid fraction, although in some instances more optional lipids could be included. In some embodiments, the optional lipids comprise less than about 10 wt %, less than about 5 wt %, or less than about 2 wt %. In some embodiments, the lipid fraction does not include optional lipids.

In a specific embodiment, the phospholipid fraction contains egg SM or palmitoyl SM or phytosphingomyelin and DPPG in a weight ratio (SM:negatively charged phospholipid) ranging from 90:10 to 99:1, more preferably ranging from 95:5 to 98:2. In one embodiment, the weight ratio is 97:3.

The molar ratio of the lipid component to the protein component of complexes of the disclosure can vary, and will depend upon, among other factors, the identity(ies) of the apolipoprotein comprising the protein component, the identities and quantities of the lipids comprising the lipid component, and the desired size of the complex. Because the biological activity of apolipoproteins such as ApoA-I are thought to be mediated by the amphipathic helices comprising the apolipoprotein, it is convenient to express the apolipoprotein fraction of the lipid:apolipoprotein molar ratio using ApoA-I protein equivalents. It is generally accepted that ApoA-I contains 6-10 amphipathic helices, depending upon the method used to calculate the helices. Other apolipoproteins can be expressed in terms of ApoA-I equivalents based upon the number of amphipathic helices they contain. For example, ApoA-IM, which typically exists as a disulfide-bridged dimer, can be expressed as 2 ApoA-I equivalents, because each molecule of ApoA-I_{M} contains twice as many amphipathic helices as a molecule of ApoA-I. Conversely, a peptide apolipoprotein that contains a single amphipathic helix can be expressed as a 1/10-⅙ ApoA-I equivalent, because each molecule contains 1/10-⅙ as many amphipathic helices as a molecule of ApoA-I. In general, the lipid:ApoA-I equivalent molar ratio of the lipoprotein complexes (defined herein as “Ri”) will range from about 105:1 to 110:1. In some embodiments, the Ri is about 108:1. Ratios in weight can be obtained using a MW of approximately 650-800 for phospholipids.

In some embodiments, the molar ratio of lipid:ApoA-I equivalents (“RSM”) ranges from about 80:1 to about 110:1, e.g., about 80:1 to about 100:1. In a specific example, the RSM for complexes can be about 82:1.

In some embodiments, lipoprotein complexes used in the methods of the disclosure are negatively charged complexes which comprise a protein fraction which is preferably mature, full-length ApoA-I, and a lipid fraction comprising a neutral phospholipid, sphingomyelin (SM), and negatively charged phospholipid.

In a specific embodiment, the lipid component contains SM (e.g., egg SM, palmitoyl SM, phytoSM, or a combination thereof) and negatively charged phospholipid (e.g., DPPG) in a weight ratio (SM:negatively charged phospholipid) ranging from 90:10 to 99:1, more preferably ranging from 95:5 to 98:2, e.g., 97:3.

In specific embodiments, the ratio of the protein component to lipid component can range from about 1:2.7 to about 1:3, with 1:2.7 being preferred. This corresponds to molar ratios of ApoA-I protein to lipid ranging from approximately 1:90 to 1:140. In some embodiments, the molar ratio of protein to lipid in the complex is about 1:90 to about 1:120, about 1:100 to about 1:140, or about 1:95 to about 1:125.

In particular embodiments, the complex comprises CER-001, CSL-111, CSL-112, CER-522 or ETC-216. In a preferred embodiment, the complex is CER-001.

CER-001 as used in the literature and in the Examples below refers to a complex described in Example 4 of WO 2012/109162. WO 2012/109162 refers to CER-001 as a complex having a 1:2.7 lipoprotein weight:total phospholipid weight ratio with a SM:DPPG weight:weight ratio of 97:3. Example 4 of WO 2012/109162 also describes a method of its manufacture.

When used in the context of a method and/or CER-001 dosing regimen of the disclosure, CER-001 refers to a lipoprotein complex whose individual constituents can vary from CER-001 as described in Example 4 of WO 2012/109162 by up to 20%. In certain embodiments, the constituents of the lipoprotein complex vary from CER-001 as described in Example 4 of WO 2012/109162 by up to 10%. Preferably, the constituents of the lipoprotein complex are those described in Example 4 of WO 2012/109162 (plus/minus acceptable manufacturing tolerance variations). The SM in CER-001 can be natural or synthetic. In some embodiments, the SM is a natural SM, for example a natural SM described in WO 2012/109162, e.g., chicken egg SM. In some embodiments, the SM is a synthetic SM, for example a synthetic SM described in WO 2012/109162, e.g., synthetic palmitoylsphingomyelin, for example as described in WO 2012/109162. Methods for synthesizing palmitoylsphingomyelin are known in the art, for example as described in WO 2014/140787. The lipoprotein in CER-001, apolipoprotein A-I (ApoA-I), preferably has an amino acid sequence corresponding to amino acids 25 to 267 of SEQ ID NO:1 of WO 2012/109162 (identical to SEQ ID NO:2 of this application). ApoA-I can be purified by animal sources (and in particular from human sources) or produced recombinantly. In preferred embodiments, the ApoA-I in CER-001 is recombinant ApoA-I. CER-001 used in a dosing regimen of the disclosure is preferably highly homogeneous, for example at least 80%, at least 85%, at least 90%, at least 95%, at least 97%, at least 98%, or at least 99% homogeneous, as reflected by a single peak in gel permeation chromatography. See, e.g., Section 6.4 of WO 2012/109162.

CSL-111 is a reconstituted human ApoA-I purified from plasma complexed with soybean phosphatidylcholine (SBPC) (Tardif et al., 2007, JAMA 297:1675-1682).

CSL-112 is a formulation of ApoA-I purified from plasma and reconstituted to form HDL suitable for intravenous infusion (Diditchenko et al., 2013, DOI 10.1161/ATVBAHA.113.301981).

ETC-216 (also known as MDCO-216) is a lipid-depleted form of HDL containing recombinant ApoA-I_{Milano}. See Nicholls et al., 2011, Expert Opin Biol Ther. 11(3):387-94. doi: 10.1517/14712598.2011.557061.

In another embodiment, a complex that can be used in the methods of the disclosure is CER-522. CER-522 is a lipoprotein complex comprising a combination of three phospholipids and a 22 amino acid peptide, CT80522:

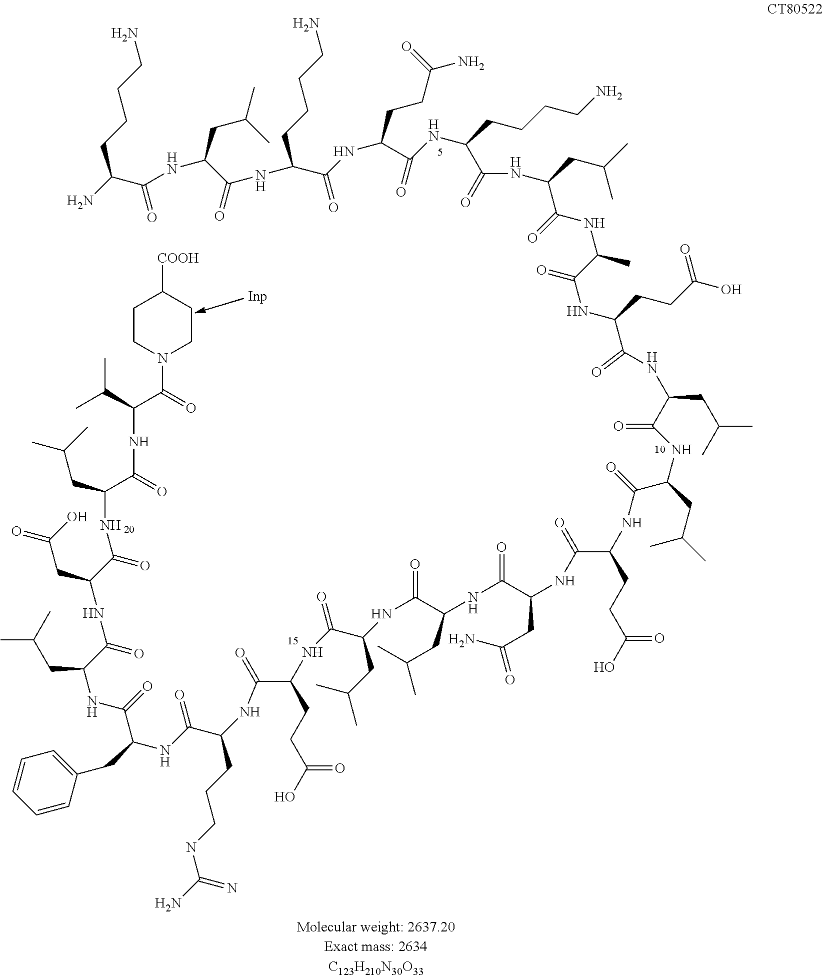 [SMILES: CC(C)C[C@H](NC(=O)[C@H](CCCCN)NC(=O)[C@H](CCC(N)=O)NC(=O)[C@H](CCCCN)NC(=O)[C@H](CC(C)C)NC(=O)[C@@H](N)CCCCN)C(=O)N[C@@H](C)C(=O)N[C@@H](CCC(=O)O)C(=O)N[C@@H](CC(C)C)C(=O)N[C@@H](CC(C)C)C(=O)N[C@@H](CCC(=O)O)C(=O)N[C@@H](CC(N)=O)C(=O)N[C@@H](CC(C)C)C(=O)N[C@@H](CC(C)C)C(=O)N[C@@H](CCC(=O)O)C(=O)N[C@@H](CCCNC(=N)N)C(=O)N[C@@H](Cc1ccccc1)C(=O)N[C@@H](CC(C)C)C(=O)N[C@@H](CC(=O)O)C(=O)N[C@@H](CC(C)C)C(=O)N[C@H](C(=O)N1CCC(C(=O)O)CC1)C(C)C]

The phospholipid component of CER-522 consists of egg sphingomyelin,1,2-dipalmitoyl-sn-glycero-3-phosphocholine (Dipalmitoylphosphatidylcholine, DPPC) and 1,2-dipalmitoyl-sn-glycero-3-[phospho-rac-(1-glycerol)](Dipalmitoylphosphatidyl-glycerol, DPPG) in a 48.5:48.5:3 weight ratio. The ratio of peptide to total phospholipids in the CER-522 complex is 1:2.5 (w/w).

In some embodiments, the lipoprotein complex is delipidated HDL. Most HDL in plasma is cholesterol-rich. The lipids in HDL can be depleted, for example partially and/or selectively depleted, e.g., to reduce its cholesterol content. In some embodiments, the delipidated HDL can resemble small a, prep-1, and other prep forms of HDL. A process for selective depletion of HDL is described in Sacks et al., 2009, J Lipid Res. 50(5): 894-907.

In certain embodiments, a lipoprotein complex comprises a bioactive agent delivery particle as described in US 2004/0229794.

A bioactive agent delivery particle can comprise a lipid binding polypeptide (e.g., an apolipoprotein as described previously in this Section or in Section 5.1.2), a lipid bilayer (e.g., comprising one or more phospholipids as described previously in this Section or in Section 5.1.4.1), and a bioactive agent (e.g., an anti-cancer agent), wherein the interior of the lipid bilayer comprises a hydrophobic region, and wherein the bioactive agent is associated with the hydrophobic region of the lipid bilayer. In some embodiments, a bioactive agent delivery particle as described in US 2004/0229794.

In some embodiments, a bioactive agent delivery particle does not comprise a hydrophilic core.

In some embodiments, a bioactive agent delivery particle is disc shaped (e.g., having a diameter from about 7 to about 29 nm).

Bioactive agent delivery particles include bilayer-forming lipids, for example phospholipids (e.g., as described previously in this Section or in Section 5.1.4.1). In some embodiments, a bioactive agent delivery particle includes both bilayer-forming and non-bilayer-forming lipids. In some embodiments, the lipid bilayer of a bioactive agent delivery particle includes phospholipids. In one embodiment, the phospholipids incorporated into a delivery particle include dimyristoylphosphatidylcholine (DMPC) and dimyristoylphosphatidylglycerol (DMPG). In one embodiment, the lipid bilayer includes DMPC and DMPG in a 7:3 molar ratio.

In some embodiments, the lipid binding polypeptide is an apolipoprotein (e.g., as described previously in this Section or in Section 5.1.2). The predominant interaction between lipid binding polypeptides, e.g., apolipoprotein molecules, and the lipid bilayer is generally a hydrophobic interaction between residues on a hydrophobic face of an amphipathic structure, e.g., an α-helix of the lipid binding polypeptide and fatty acyl chains of lipids on an exterior surface at the perimeter of the particle. Bioactive agent delivery particles may include exchangeable and/or non-exchangeable apolipoproteins. In one embodiment, the lipid binding polypeptide is ApoA-I.

In some embodiments, bioactive agent delivery particles include lipid binding polypeptide molecules, e.g., apolipoprotein molecules, that have been modified to increase stability of the particle. In one embodiment, the modification includes introduction of cysteine residues to form intramolecular and/or intermolecular disulfide bonds.

In another embodiment, bioactive agent delivery particles include a chimeric lipid binding polypeptide molecule, e.g., a chimeric apolipoprotein molecule, with one or more bound functional moieties, for example one or more targeting moieties and/or one or more moieties having a desired biological activity, e.g., antimicrobial activity, which may augment or work in synergy with the activity of a bioactive agent incorporated into the delivery particle.

### 5.1.2. ApoA-I Formulations

In one aspect, the disclosure relates to an ApoA-I formulation comprising ApoA-I and one or more lipids, wherein the ApoA-I and the lipids are in the form of lipoprotein complexes.

The ApoA-I can be any such apolipoprotein described in Section 5.1.3.1, including, among others, ApoA-I having the amino acid sequence of amino acids 25-267 of SEQ ID NO:2 and/or ApoA-I that is recombinantly expressed.

The lipids can be any one or more of those described in Section 5.1.4.1. The lipids can include neutral lipids and/or negatively charged lipids. The neutral lipids can comprise sphingomyelin or consist of sphingomyelin, such as natural sphingomyelin (e.g., chicken egg sphingomyelin) and/or synthetic sphingomyelin (e.g., palmitoylsphingomyelin). The negatively charged lipids can comprise 1,2-dipalmitoyl-sn-glycero-3-[phospho-rac-(1-glycerol)](“DPPG”) or a salt thereof, or consist of DPPG or a salt thereof.

The lipids can include neutral phospholipid and negatively charged phospholipid at any weight or molar ratio described herein. The lipids can consist of 95 to 99 weight % neutral phospholipid and 1 to 5 weight % negatively charged phospholipid, such as 96 to 98 weight % neutral phospholipid and 2 to 4 weight % negatively charged phospholipid, or 97 weight % neutral phospholipid and 3 weight % negatively charged phospholipid.

The formulations can comprise ApoA-I and the lipids at any weight or molar ratio described herein. For an example, the molar ratio of the components of the negatively charged lipid to the neutral lipid to the ApoA-I in the formulation is 2-6:90-120:1. Exemplary formulations can include ApoA-I to lipid ratios ranging from 1:2 to 1:3 by weight, such as about 1:2.7 by weight.

The formulations can be used in a method of treating a subject having one or more symptoms associated with leukocytosis, having endothelial dysfunction, having or at risk of developing carditis, having or at risk of developing leukocytosis, experiencing acute coronary syndrome or stroke, and/or who has experienced acute coronary syndrome or stroke, such as the methods described at Sections 5.2-5.4. The formulations can also be for use in treating such diseases or disorders. The formulations can also be for use in the manufacture of a medicament for treating such diseases or disorders.

Examples of ApoA-I formulations and uses thereof include those set forth as numbered embodiments 1-72 of Group 2.

### 5.1.3. Lipid Binding Protein Molecules

Lipid binding protein molecules that can be used in the complexes described herein include apolipoproteins such as those described in Section 5.1.3.1 and apolipoprotein mimetic peptides such as those described in Section 5.1.3.2. In some embodiments, the complex comprises a mixture of lipid binding protein molecules. In some embodiments, the complex comprises a mixture of one or more lipid binding protein molecules and one or more apolipoprotein mimetic peptides.

In some embodiments, the complex comprises 1 to 8 ApoA-I equivalents (e.g., 1, 2, 3, 4, 5, 6, 7, 8, 1 to 7, 1 to 6, 1 to 5, 1 to 4, 1 to 3, 1 to 2, 2 to 8, 2 to 6, 2 to 4, 4 to 6, or 4 to 8 ApoA-I equivalents). Lipid binding proteins can be expressed in terms of ApoA-I equivalents based upon the number of amphipathic helices they contain. For example, ApoA-I_{M}, which typically exists as a disulfide-bridged dimer, can be expressed as 2 ApoA-I equivalents, because each molecule of ApoA-I_{M} contains twice as many amphipathic helices as a molecule of ApoA-I. Conversely, a peptide mimetic that contains a single amphipathic helix can be expressed as a 1/10-⅙ ApoA-I equivalent, because each molecule contains 1/10-⅙ as many amphipathic helices as a molecule of ApoA-I.

### 5.1.3.1. Apolipoproteins

Suitable apolipoproteins that can be included in the lipid binding protein-based complexes include apolipoproteins ApoA-I, ApoA-II, ApoA-IV, ApoA-V, ApoB, ApoC-I, ApoC-II, ApoC-III, ApoD, ApoE, ApoJ, ApoH, and any combination of two or more of the foregoing.

Polymorphic forms, isoforms, variants and mutants as well as truncated forms of the foregoing apolipoproteins, the most common of which are Apolipoprotein A-IMilano (ApoA-IM), Apolipoprotein A-IParis (ApoA-IP), and Apolipoprotein A-IZaragoza (ApoA-IZ), can also be used. Apolipoproteins mutants containing cysteine residues are also known, and can also be used (see, e.g., U.S. Publication No. 2003/0181372). The apolipoproteins may be in the form of monomers or dimers, which may be homodimers or heterodimers. For example, homo- and heterodimers (where feasible) of ApoA-I (Duverger et al., 1996, Arterioscler. Thromb. Vasc. Biol. 16(12):1424-29), ApoA-IM (Franceschini et al., 1985, J. Biol. Chem. 260:1632-35), ApoA-IP (Daum et al., 1999, J. Mol. Med. 77:614-22), ApoA-II (Shelness et al., 1985, J. Biol. Chem. 260(14):8637-46; Shelness et al., 1984, J. Biol. Chem. 259(15):9929-35), ApoA-IV (Duverger et al., 1991, Euro. J. Biochem. 201(2):373-83), ApoE (McLean et al., 1983, J. Biol. Chem. 258(14):8993-9000), ApoJ and ApoH may be used.

The apolipoproteins can be modified in their primary sequence to render them less susceptible to oxidations, for example, as described in U.S. Publication Nos. 2008/0234192 and 2013/0137628, and U.S. Pat. Nos. 8,143,224 and 8,541,236. The apolipoproteins can include residues corresponding to elements that facilitate their isolation, such as His tags, or other elements designed for other purposes. Preferably, the apolipoprotein in the complex is soluble in a biological fluid (e.g., lymph, cerebrospinal fluid, vitreous humor, aqueous humor, blood, or a blood fraction (e.g., serum or plasma).

In some embodiments, the complex comprises covalently bound lipid-binding protein monomers, e.g., dimeric apolipoprotein A-IMilano, which is a mutated form of ApoA-I containing a cysteine. The cysteine allows the formation of a disulfide bridge which can lead to the formation of homodimers or heterodimers (e.g., ApoA-I Milano-ApoA-II).

In some embodiments, the apolipoprotein molecules comprise ApoA-I, ApoA-II, ApoA-IV, ApoA-V, ApoB, ApoC-I, ApoC-II, ApoC-III, ApoD, ApoE, ApoJ, or ApoH molecules or a combination thereof.

In some embodiments, the apolipoprotein molecules comprise or consist of ApoA-I molecules. In some embodiments, said ApoA-I molecules are human ApoA-I molecules. In some embodiments, said ApoA-I molecules are recombinant. In some embodiments, the ApoA-I molecules are not ApoA-IMilano.

In some embodiments, the ApoA-I molecules are Apolipoprotein A-IMilano (ApoA-IM), Apolipoprotein A-IParis (ApoA-IP), or Apolipoprotein A-IZaragoza (ApoA-IZ) molecules.

Apolipoproteins can be purified from animal sources (and in particular from human sources) or produced recombinantly as is well-known in the art, see, e.g., Chung et al., 1980, J. Lipid Res. 21(3):284-91; Cheung et al., 1987, J. Lipid Res. 28(8):913-29. See also U.S. Pat. Nos. 5,059,528, 5,128,318, 6,617,134; U.S. Publication Nos. 2002/0156007, 2004/0067873, 2004/0077541, and 2004/0266660; and PCT Publications Nos. WO 2008/104890 and WO 2007/023476. Other methods of purification are also possible, for example as described in PCT Publication No. WO 2012/109162, the disclosure of which is incorporated herein by reference in its entirety.

The apolipoprotein can be in prepro-form, pro-form, or mature form. For example, a complex can comprise ApoA-I (e.g., human ApoA-I) in which the ApoA-I is preproApoA-I, proApoA-I, or mature ApoA-I. In some embodiments, the complex comprises ApoA-I that has at least 90% sequence identity to SEQ ID NO:1:

(SEQ ID NO: 1)
PPQSPWDRVKDLATVYVDVLKDSGRDYVSQFEGSALGKQLNLKLLDNWD
SVTSTFSKLREQLGPVTQEFWDNLEKETEGLRQEMSKDLEEVKAKVQPY
LDDFQKKWQEEMELYRQKVEPLRAELQEGARQKLHELQEKLSPLGEEMR
DRARAHVDALRTHLAPYSDELRQRLAARLEALKENGGARLAEYHAKATE
HLSTLSEKAKPALEDLRQGLLPVLESFKVSFLSALEEYTKKLNTQ

In other embodiments, the complex comprises ApoA-I that has at least 95% sequence identity to SEQ ID NO:1. In other embodiments, the complex comprises ApoA-I that has at least 98% sequence identity to SEQ ID NO:1. In other embodiments, the complex comprises ApoA-I that has at least 99% sequence identity to SEQ ID NO:1. In other embodiments, the complex comprises ApoA-I that has 100% sequence identity to SEQ ID NO:1.

In some embodiments, the complex comprises 1 to 8 apolipoprotein molecules (e.g., 1 to 6, 1 to 4, 1 to 2, 2 to 8, 2 to 6, 2 to 4, 4 to 8, 4 to 6, or 6 to 8 apolipoprotein molecules). In some embodiments, the complex comprises 1 apolipoprotein molecule. In some embodiments, the complex comprises 2 apolipoprotein molecules. In some embodiments, the complex comprises 3 apolipoprotein molecules. In some embodiments, the complex comprises 4 apolipoprotein molecules. In some embodiments, the complex comprises 5 apolipoprotein molecules. In some embodiments, the complex comprises 6 apolipoprotein molecules. In some embodiments, the complex comprises 7 apolipoprotein molecules. In some embodiments, the complex comprises 8 apolipoprotein molecules.

The apolipoprotein molecule(s) can comprise a chimeric apolipoprotein comprising an apolipoprotein and one or more attached functional moieties, such as for example, one or more CRN-001 complex(es), one or more targeting moieties, a moiety having a desired biological activity, an affinity tag to assist with purification, and/or a reporter molecule for characterization or localization studies. An attached moiety with biological activity may have an activity that is capable of augmenting and/or synergizing with the biological activity of a compound incorporated into a complex of the disclosure. For example, a moiety with biological activity may have antimicrobial (for example, antifungal, antibacterial, anti-protozoal, bacteriostatic, fungistatic, or antiviral) activity. In one embodiment, an attached functional moiety of a chimeric apolipoprotein is not in contact with hydrophobic surfaces of the complex. In another embodiment, an attached functional moiety is in contact with hydrophobic surfaces of the complex. In some embodiments, a functional moiety of a chimeric apolipoprotein may be intrinsic to a natural protein. In some embodiments, a chimeric apolipoprotein includes a ligand or sequence recognized by or capable of interaction with a cell surface receptor or other cell surface moiety.

In one embodiment, a chimeric apolipoprotein includes a targeting moiety that is not intrinsic to the native apolipoprotein, such as for example, S. cerevisiae α-mating factor peptide, folic acid, transferrin, or lactoferrin. In another embodiment, a chimeric apolipoprotein includes a moiety with a desired biological activity that augments and/or synergizes with the activity of a compound incorporated into a complex of the disclosure. In one embodiment, a chimeric apolipoprotein may include a functional moiety intrinsic to an apolipoprotein. One example of an apolipoprotein intrinsic functional moiety is the intrinsic targeting moiety formed approximately by amino acids 130-150 of human ApoE, which comprises the receptor binding region recognized by members of the low density lipoprotein receptor family. Other examples of apolipoprotein intrinsic functional moieties include the region of ApoB-100 that interacts with the low density lipoprotein receptor and the region of ApoA-I that interacts with scavenger receptor type B 1. In other embodiments, a functional moiety may be added synthetically or recombinantly to produce a chimeric apolipoprotein. Another example is an apolipoprotein with the prepro or pro sequence from another preproapolipoprotein (e.g., prepro sequence from preproapoA-II substituted for the prepro sequence of preproapoA-I). Another example is an apolipoprotein for which some of the amphipathic sequence segments have been substituted by other amphipathic sequence segments from another apolipoprotein.

As used herein, “chimeric” refers to two or more molecules that are capable of existing separately and are joined together to form a single molecule having the desired functionality of all of its constituent molecules. The constituent molecules of a chimeric molecule may be joined synthetically by chemical conjugation or, where the constituent molecules are all polypeptides or analogs thereof, polynucleotides encoding the polypeptides may be fused together recombinantly such that a single continuous polypeptide is expressed. Such a chimeric molecule is termed a fusion protein. A “fusion protein” is a chimeric molecule in which the constituent molecules are all polypeptides and are attached (fused) to each other such that the chimeric molecule forms a continuous single chain. The various constituents can be directly attached to each other or can be coupled through one or more linkers. One or more segments of various constituents can be, for example, inserted in the sequence of an apolipoprotein, or, as another example, can be added N-terminal or C-terminal to the sequence of an apolipoprotein. For example, a fusion protein can comprise an antibody light chain, an antibody fragment, a heavy-chain antibody, or a single-domain antibody.

In some embodiments, a chimeric apolipoprotein is prepared by chemically conjugating the apolipoprotein and the functional moiety to be attached. Means of chemically conjugating molecules are well known to those of skill in the art. Such means will vary according to the structure of the moiety to be attached, but will be readily ascertainable to those of skill in the art. Polypeptides typically contain a variety of functional groups, e.g., carboxylic acid (—COOH), free amino (—NH2), or sulfhydryl (—SH) groups, that are available for reaction with a suitable functional group on the functional moiety or on a linker to bind the moiety thereto. A functional moiety may be attached at the N-terminus, the C-terminus, or to a functional group on an interior residue (i.e., a residue at a position intermediate between the N- and C-termini) of an apolipoprotein molecule. Alternatively, the apolipoprotein and/or the moiety to be tagged can be derivatized to expose or attach additional reactive functional groups.

In some embodiments, fusion proteins that include a polypeptide functional moiety are synthesized using recombinant expression systems. Typically, this involves creating a nucleic acid (e.g., DNA) sequence that encodes the apolipoprotein and the functional moiety such that the two polypeptides will be in frame when expressed, placing the DNA under the control of a promoter, expressing the protein in a host cell, and isolating the expressed protein.

A nucleic acid encoding a chimeric apolipoprotein can be incorporated into a recombinant expression vector in a form suitable for expression in a host cell. As used herein, an “expression vector” is a nucleic acid which, when introduced into an appropriate host cell, can be transcribed and translated into a polypeptide. The vector may also include regulatory sequences such as promoters, enhancers, or other expression control elements (e.g., polyadenylation signals). Such regulatory sequences are known to those skilled in the art (see, e.g., Goeddel, 1990, Gene Expression Technology: Meth. Enzymol. 185, Academic Press, San Diego, Calif.; Berger and Kimmel, Guide to Molecular Cloning Techniques, Methods in Enzymology 152 Academic Press, Inc., San Diego, Calif.; Sambrook et al., 1989, Molecular Cloning—A Laboratory Manual (2nd ed.) Vol. 1-3, Cold Spring Harbor Laboratory, Cold Spring Harbor Press, NY, etc.).

In some embodiments, an apolipoprotein has been modified such that when the apolipoprotein is incorporated into a complex of the disclosure, the modification will increase stability of the complex, confer targeting ability or increase capacity. In one embodiment, the modification includes introduction of cysteine residues into apolipoprotein molecules to permit formation of intramolecular or intermolecular disulfide bonds, e.g., by site-directed mutagenesis. In another embodiment, a chemical crosslinking agent is used to form intermolecular links between apolipoprotein molecules to enhance stability of the complex. Intermolecular crosslinking prevents or reduces dissociation of apolipoprotein molecules from the complex and/or prevents displacement by endogenous apolipoprotein molecules within an individual to whom the complexes are administered. In other embodiments, an apolipoprotein is modified either by chemical derivatization of one or more amino acid residues or by site directed mutagenesis, to confer targeting ability to or recognition by a cell surface receptor.

Complexes can be targeted to a specific cell surface receptor by engineering receptor recognition properties into an apolipoprotein. For example, complexes may be targeted to a particular cell type known to harbor a particular type of infectious agent, for example by modifying the apolipoprotein to render it capable of interacting with a receptor on the surface of the cell type being targeted. For example, complexes may be targeted to macrophages by altering the apolipoprotein to confer recognition by the macrophage endocytic class A scavenger receptor (SR-A). SR-A binding ability can be conferred to a complex by modifying the apolipoprotein by site directed mutagenesis to replace one or more positively charged amino acids with a neutral or negatively charged amino acid. SR-A recognition can also be conferred by preparing a chimeric apolipoprotein that includes an N- or C-terminal extension having a ligand recognized by SR-A or an amino acid sequence with a high concentration of negatively charged residues. Complexes comprising apoplipoproteins can also interact with apolipoprotein receptors such as, but not limited to, ABCA1 receptors, ABCG1 receptors, Megalin, Cubulin and HDL receptors such as SR-B1.

### 5.1.3.2. Apolipoprotein Mimetics

Peptides, peptide analogs, and agonists that mimic the activity of an apolipoprotein (collectively referred to herein as “apolipoprotein peptide mimetics”) can also be used in the complexes described herein, either alone, in combination with one or more other lipid binding proteins. Non-limiting examples of peptides and peptide analogs that correspond to apolipoproteins, as well as agonists that mimic the activity of ApoA-I, ApoA-I_{M}, ApoA-II, ApoA-IV, and ApoE, that are suitable for inclusion in the complexes and compositions described herein are disclosed in U.S. Pat. Nos. 6,004,925, 6,037,323 and 6,046,166 (issued to Dasseux et al.), U.S. Pat. No. 5,840,688 (issued to Tso), U.S. Pat. No. 6,743,778 (issued to Kohno), U.S. Publication Nos. 2004/0266671, 2004/0254120, 2003/0171277 and 2003/0045460 (to Fogelman), U.S. Publication No. 2006/0069030 (to Bachovchin), U.S. Publication No. 2003/0087819 (to Bielicki), U.S. Publication No. 2009/0081293 (to Murase et al.), and PCT Publication No. WO/2010/093918 (to Dasseux et al.), the disclosures of which are incorporated herein by reference in their entireties. These peptides and peptide analogues can be composed of L-amino acid or D-amino acids or mixture of L- and D-amino acids. They may also include one or more non-peptide or amide linkages, such as one or more well-known peptide/amide isosteres. Such apolipoprotein peptide mimetic can be synthesized or manufactured using any technique for peptide synthesis known in the art, including, e.g., the techniques described in U.S. Pat. Nos. 6,004,925, 6,037,323 and 6,046,166.

In some embodiments, the lipid binding protein molecules comprise apolipoprotein peptide mimetic molecules and optionally one or more apolipoprotein molecules such as those described above.

In some embodiments, the apolipoprotein peptide mimetic molecules comprise an ApoA-I peptide mimetic, ApoA-II peptide mimetic, ApoA-IV peptide mimetic, or ApoE peptide mimetic or a combination thereof.

### 5.1.4. Amphipathic Molecules

An amphipathic molecule is a molecule that possesses both hydrophobic (apolar) and hydrophilic (polar) elements. Amphipathic molecules that can be used in complexes described herein include lipids (e.g., as described in Section 5.1.4.1), detergents (e.g., as described in Section 5.1.4.2), fatty acids (e.g., as described in Section 5.1.4.3), and apolar molecules and sterols covalently attached to polar molecules such as, but not limited to, sugars or nucleic acids (e.g., as described in Section 5.1.4.4).

The complexes can include a single class of amphipathic molecule (e.g., a single species of phospholipids or a mixture of phospholipids) or can contain a combination of classes of amphipathic molecules (e.g., phospholipids and detergents). The complex can contain one species of amphipathic molecules or a combination of amphipathic molecules configured to facilitate solubilization of the lipid binding protein molecule(s).

In some embodiments, the amphipathic molecules included in comprise a phospholipid, a detergent, a fatty acid, an apolar moiety or sterol covalently attached to a sugar, or a combination thereof (e.g., selected from the types of amphipathic molecules discussed above).

In some embodiments, the amphipathic molecules comprise or consist of phospholipid molecules. In some embodiments, the phospholipid molecules comprise negatively charged phospholipids, neutral phospholipids, positively charged phospholipids or a combination thereof. In some embodiments, the phospholipid molecules contribute a net charge of 1-3 per apolipoprotein molecule in the complex. In some embodiments, the net charge is a negative net charge. In some embodiments, the net charge is a positive net charge. In some embodiments, the phospholipid molecules consist of a combination of negatively charged and neutral phospholipids. In some embodiments, the molar ratio of negatively charge phospholipid to neutral phospholipid ranges from 1:1 to 1:3. In some embodiments, the molar ratio of negatively charged phospholipid to neutral phospholipid is about 1:1 or about 1:2.

In some embodiments, the amphipathic molecules comprise neutral phospholipids and negatively charged phospholipids in a weight ratio of 95:5 to 99:1.

### 5.1.4.1. Lipids

Lipid binding protein-based complexes can include one or more lipids. In various embodiments, one or more lipids can be saturated and/or unsaturated, natural and/or synthetic, charged or not charged, zwitterionic or not. In some embodiments, the lipid molecules (e.g., phospholipid molecules) can together contribute a net charge of 1-3 (e.g., 1-3, 1-2, 2-3, 1, 2, or 3) per lipid binding protein molecule in the complex. In some embodiments, the net charge is negative. In other embodiments, the net charge is positive.

In some embodiments, the lipid comprises a phospholipid. Phospholipids can have two acyl chains that are the same or different (for example, chains having a different number of carbon atoms, a different degree of saturation between the acyl chains, different branching of the acyl chains, or a combination thereof). The lipid can also be modified to contain a fluorescent probe (e.g., as described at avantilipids.com/product-category/products/fluorescent-lipids/). Preferably, the lipid comprises at least one phospholipid.

Phospholipids can have unsaturated or saturated acyl chains ranging from about 6 to about 24 carbon atoms (e.g., 6-20, 6-16, 6-12, 12-24, 12-20, 12-16, 16-24, 16-20, or 20-24). In some embodiments, a phospholipid used in a complex of the disclosure has one or two acyl chains of 12, 14, 16, 18, 20, 22, or 24 carbons (e.g., two acyl chains of the same length or two acyl chains of different length).

Non-limiting examples of acyl chains present in commonly occurring fatty acids that can be included in phospholipids are provided in Table 1, below:

TABLE 1

| Length:Number of Unsaturations | Common Name |
|---|---|
| 14:0 | myristic acid |
| 16:0 | palmitic acid |
| 18:0 | stearic acid |
| 18:1 cisΔ⁹ | oleic acid |
| 18:2 cisΔ^{9, 12} | linoleic acid |
| 18:3 cisΔ^{9, 12, 15} | linonenic acid |
| 20:4 cisΔ^{5, 8, 11, 14} | arachidonic acid |
| 20:5 cisΔ^{5, 8, 11, 14, 17} | eicosapentaenoic acid |
| | (an omega-3 fatty acid) |

Lipids that can be present in the complexes of the disclosure include, but are not limited to, small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, phosphatidylglycerols, diphosphatidylglycerols such as dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, palmitoylsphingomyelin, dipalmitoylsphingomyelin, egg sphingomyelin, milk sphingomyelin, phytosphingomyelin, distearoylsphingomyelin, dipalmitoylphosphatidylglycerol salt, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives. Synthetic lipids, such as synthetic palmitoylsphingomyelin or N-palmitoyl-4-hydroxysphinganine-1-phosphocholine (a form of phytosphingomyelin) can be used to minimize lipid oxidation.

In some embodiments, a lipid binding protein-based complex includes two types of phospholipids: a neutral lipid, e.g., lecithin and/or sphingomyelin (abbreviated SM), and a charged phospholipid (e.g., a negatively charged phospholipid). A “neutral” phospholipid has a net charge of about zero at physiological pH. In many embodiments, neutral phospholipids are zwitterions, although other types of net neutral phospholipids are known and can be used. In some embodiments, the molar ratio of the charged phospholipid (e.g., negatively charged phospholipid) to neutral phospholipid ranges from 1:1 to 1:3, for example, about 1:1, about 1:2, or about 1:3.

The neutral phospholipid can comprise, for example, one or both of the lecithin and/or SM, and can optionally include other neutral phospholipids. In some embodiments, the neutral phospholipid comprises lecithin, but not SM. In other embodiments, the neutral phospholipid comprises SM, but not lecithin. In still other embodiments, the neutral phospholipid comprises both lecithin and SM. All of these specific exemplary embodiments can include neutral phospholipids in addition to the lecithin and/or SM, but in many embodiments do not include such additional neutral phospholipids.

As used herein, the expression “SM” includes sphingomyelins derived or obtained from natural sources, as well as analogs and derivatives of naturally occurring SMs that are impervious to hydrolysis by LCAT, as is naturally occurring SM. SM is a phospholipid very similar in structure to lecithin, but, unlike lecithin, it does not have a glycerol backbone, and hence does not have ester linkages attaching the acyl chains. Rather, SM has a ceramide backbone, with amide linkages connecting the acyl chains. SM can be obtained, for example, from milk, egg or brain. SM analogues or derivatives can also be used. Non-limiting examples of useful SM analogues and derivatives include, but are not limited to, palmitoylsphingomyelin, N-palmitoyl-4-hydroxysphinganine-1-phosphocholine (a form of phytosphingomyelin), palmitoylsphingomyelin, stearoylsphingomyelin, D-erythro-N-16:0-sphingomyelin and its dihydro isomer, D-erythro-N-16:0-dihydro-sphingomyelin. Synthetic SM such as synthetic palmitoylsphingomyelin or N-palmitoyl-4-hydroxysphinganine-1-phosphocholine (phytosphingomyelin) can be used in order to produce more homogeneous complexes and with fewer contaminants and/or oxidation products than sphingolipids of animal origin. Methods for synthesizing SM are described in U.S. Publication No. 2016/0075634.

Sphingomyelins isolated from natural sources can be artificially enriched in one particular saturated or unsaturated acyl chain. For example, milk sphingomyelin (Avanti Phospholipid, Alabaster, Ala.) is characterized by long saturated acyl chains (i.e., acyl chains having 20 or more carbon atoms). In contrast, egg sphingomyelin is characterized by short saturated acyl chains (i.e., acyl chains having fewer than 20 carbon atoms). For example, whereas only about 20% of milk sphingomyelin comprises C16:0 (16 carbon, saturated) acyl chains, about 80% of egg sphingomyelin comprises C16:0 acyl chains. Using solvent extraction, the composition of milk sphingomyelin can be enriched to have an acyl chain composition comparable to that of egg sphingomyelin, or vice versa.

The SM can be semi-synthetic such that it has particular acyl chains. For example, milk sphingomyelin can be first purified from milk, then one particular acyl chain, e.g., the C16:0 acyl chain, can be cleaved and replaced by another acyl chain. The SM can also be entirely synthesized, by e.g., large-scale synthesis. See, e.g., Dong et al., U.S. Pat. No. 5,220,043, entitled Synthesis of D-erythro-sphingomyelins, issued Jun. 15, 1993; Weis, 1999, Chem. Phys. Lipids 102 (1-2):3-12. SM can be fully synthetic, e.g., as described in U.S. Publication No. 2014/0275590.

The lengths and saturation levels of the acyl chains comprising a semi-synthetic or a synthetic SM can be selectively varied. The acyl chains can be saturated or unsaturated, and can contain from about 6 to about 24 carbon atoms. Each chain can contain the same number of carbon atoms or, alternatively each chain can contain different numbers of carbon atoms. In some embodiments, the semi-synthetic or synthetic SM comprises mixed acyl chains such that one chain is saturated and one chain is unsaturated. In such mixed acyl chain SMs, the chain lengths can be the same or different. In other embodiments, the acyl chains of the semi-synthetic or synthetic SM are either both saturated or both unsaturated. Again, the chains can contain the same or different numbers of carbon atoms. In some embodiments, both acyl chains comprising the semi-synthetic or synthetic SM are identical. In a specific embodiment, the chains correspond to the acyl chains of a naturally-occurring fatty acid, such as for example oleic, palmitic or stearic acid. In another embodiment, SM with saturated or unsaturated functionalized chains is used. In another specific embodiment, both acyl chains are saturated and contain from 6 to 24 carbon atoms. Non-limiting examples of acyl chains present in commonly occurring fatty acids that can be included in semi-synthetic and synthetic SMs are provided in Table 1, above.

In some embodiments, the SM is palmitoyl SM, such as synthetic palmitoyl SM, which has C16:0 acyl chains, or is egg SM, which includes as a principal component palmitoyl SM.

In a specific embodiment, functionalized SM, such as phytosphingomyelin, is used.

Lecithin can be derived or isolated from natural sources, or it can be obtained synthetically. Examples of suitable lecithins isolated from natural sources include, but are not limited to, egg phosphatidylcholine and soybean phosphatidylcholine. Additional non-limiting examples of suitable lecithins include, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, 1-palmitoyl-2-oleoylphosphatidylcholine, 1-oleoyl-2-palmitylphosphatidylcholine, dioleoylphosphatidylcholine and the ether derivatives or analogs thereof.

Lecithins derived or isolated from natural sources can be enriched to include specified acyl chains. In embodiments employing semi-synthetic or synthetic lecithins, the identity(ies) of the acyl chains can be selectively varied, as discussed above in connection with SM. In some embodiments of the complexes described herein, both acyl chains on the lecithin are identical. In some embodiments of complexes that include both SM and lecithin, the acyl chains of the SM and lecithin are all identical. In a specific embodiment, the acyl chains correspond to the acyl chains of myristitic, palmitic, oleic or stearic acid.

The complexes of the disclosure can include one or more negatively charged phospholipids (e.g., alone or in combination with one or more neutral phospholipids). As used herein, “negatively charged phospholipids” are phospholipids that have a net negative charge at physiological pH. The negatively charged phospholipid can comprise a single type of negatively charged phospholipid, or a mixture of two or more different, negatively charged, phospholipids. In some embodiments, the charged phospholipids are negatively charged glycerophospholipids. Specific examples of suitable negatively charged phospholipids include, but are not limited to, a 1,2-dipalmitoyl-sn-glycero-3-[phospho-rac-(1-glycerol)], a phosphatidylglycerol, a phospatidylinositol, a phosphatidylserine, a phosphatidic acid, and salts thereof (e.g., sodium salts or potassium salts). In some embodiments, the negatively charged phospholipid comprises one or more of phosphatidylinositol, phosphatidylserine, phosphatidylglycerol and/or phosphatidic acid. In a specific embodiment, the negatively charged phospholipid comprises or consists of a salt of a phosphatidylglycerol or a salt of a phosphatidylinositol. In another specific embodiment, the negatively charged phospholipid comprises or consists of 1,2-dipalmitoyl-sn-glycero-3-[phospho-rac-(1-glycerol)], or DPPG, or a salt thereof.

The negatively charged phospholipids can be obtained from natural sources or prepared by chemical synthesis. In embodiments employing synthetic negatively charged phospholipids, the identities of the acyl chains can be selectively varied, as discussed above in connection with SM. In some embodiments of the complexes of the disclosure, both acyl chains on the negatively charged phospholipids are identical. In some embodiments, the acyl chains all types of phospholipids included in a complex of the disclosure are all identical. In a specific embodiment, the complex comprises negatively charged phospholipid(s), and/or SM all having C16:0 or C16:1 acyl chains. In a specific embodiment the fatty acid moiety of the SM is predominantly C16:1 palmitoyl. In one specific embodiment, the acyl chains of the charged phospholipid(s), lecithin and/or SM correspond to the acyl chain of palmitic acid. In yet another specific embodiment, the acyl chains of the charged phospholipid(s), lecithin and/or SM correspond to the acyl chain of oleic acid.

Examples of positively charged phospholipids that can be included in the complexes of the disclosure include N1-[2-((1S)-1-[(3-aminopropyl)amino]-4-[di(3-amino-propyl)amino]butylcarboxamido)ethyl]-3,4-di[oleyloxy]-benzamide, 1,2-di-O-octadecenyl-3-trimethylammonium propane, 1,2-dimyristoleoyl-sn-glycero-3-ethylphosphocholine, 1-palmitoyl-2-oleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, 1,2-dilauroyl-sn-glycero-3-ethylphosphocholine, 1,2-dioleoyl-3-dimethylammonium-propane1,2-dimyristoyl-3-dimethylammonium-propane, 1,2-dipalmitoyl-3-dimethylammonium-propane, N-(4-carboxybenzyl)-N,N-dimethyl-2,3-bis(oleoyloxy)propan-1-aminium, 1,2-dioleoyl-3-trimethylammonium-propane, 1,2-dioleoyl-3-trimethylammonium-propane, 1,2-stearoyl-3-trimethylammonium-propane, 1,2-dipalmitoyl-3-trimethylammonium-propane, 1,2-dimyristoyl-3-trimethylammonium-propane, N-[1-(2,3-dimyristyloxy)propyl]-N, N-dimethyl-N-(2-hydroxyethyl) ammonium bromide, N,N,N-trimethyl-2-bis[(1-oxo-9-octadecenyl)oxy]-(Z,Z)-1propanaminium methyl sulfate, and salts thereof (e.g., chloride or bromide salts).

The lipids used are preferably at least 95% pure, and/or have reduced levels of oxidative agents (such as but not limited to peroxides). Lipids obtained from natural sources preferably have fewer polyunsaturated fatty acid moieties and/or fatty acid moieties that are not susceptible to oxidation. The level of oxidation in a sample can be determined using an iodometric method, which provides a peroxide value, expressed in milli-equivalent number of isolated iodines per kg of sample, abbreviated meq O/kg. See, e.g., Gray, 1978, Measurement of Lipid Oxidation: A Review, Journal of the American Oil Chemists Society 55:539-545; Heaton, F. W. and Ur, Improved Iodometric Methods for the Determination of Lipid Peroxides, 1958, Journal of the Science of Food and Agriculture 9:781-786. Preferably, the level of oxidation, or peroxide level, is low, e.g., less than 5 meq O/kg, less than 4 meq O/kg, less than 3 meq O/kg, or less than 2 meq O/kg.

Complexes can in some embodiments include small quantities of additional lipids. Virtually any type of lipids can be used, including, but not limited to, lysophospholipids, galactocerebroside, gangliosides, cerebrosides, glycerides, triglycerides, and sterols and sterol derivatives (e.g., a plant sterol, an animal sterol, such as cholesterol, or a sterol derivative, such as a cholesterol derivative). For example, a complex of the disclosure can contain cholesterol or a cholesterol derivative, e.g., a cholesterol ester. The cholesterol derivative can also be a substituted cholesterol or a substituted cholesterol ester. The complexes of the disclosure can also contain an oxidized sterol such as, but not limited to, oxidized cholesterol or an oxidized sterol derivative (such as, but not limited to, an oxidized cholesterol ester). In some embodiments, the complexes do not include cholesterol and/or its derivatives (such as a cholesterol ester or an oxidized cholesterol ester).

### 5.1.4.2. Detergents

The complexes can contain one or more detergents. The detergent can be zwitterionic, nonionic, cationic, anionic, or a combination thereof. Exemplary zwitterionic detergents include 3-[(3-Cholamidopropyl)dimethylammonio]-1-propanesulfonate (CHAPS), 3-[(3-Cholamidopropyl)dimethylammonio]-2-hydroxy-1-propanesulfonate (CHAPSO), and N,N-dimethyldodecylamine N-oxide (LDAO). Exemplary nonionic detergents include D-(+)-trehalose 6-monooleate, N-octanoyl-N-methylglucamine, N-nonanoyl-N-methylglucamine, N-decanoyl-N-methylglucamine, 1-(7Z-hexadecenoyl)-rac-glycerol, 1-(8Z-hexadecenoyl)-rac-glycerol, 1-(8Z-heptadecenoyl)-rac-glycerol, 1-(9Z-hexadecenoyl)-rac-glycerol, 1-decanoyl-rac-glycerol. Exemplary cationic detergents include (S)-O-methyl-serine dodecylamide hydrochloride, dodecylammonium chloride, decyltrimethylammonium bromide, and cetyltrimethylammonium sulfate. Exemplary anionic detergents include cholesteryl hemisuccinate, cholate, alkyl sulfates, and alkyl sulfonates.

### 5.1.4.3. Fatty Acids

The complexes can contain one or more fatty acids. The one or more fatty acids can include short-chain fatty acids having aliphatic tails of five or fewer carbons (e.g. butyric acid, isobutyric acid, valeric acid, or isovaleric acid), medium-chain fatty acids having aliphatic tails of 6 to 12 carbons (e.g., caproic acid, caprylic acid, capric acid, or lauric acid), long-chain fatty acids having aliphatic tails of 13 to 21 carbons (e.g., myristic acid, palmitic acid, stearic acid, or arachidic acid), very long chain fatty acids having aliphatic tails of 22 or more carbons (e.g., behenic acid, lignoceric acid, or cerotic acid), or a combination thereof. The one or more fatty acids can be saturated (e.g., caprylic acid, capric acid, lauric acid, myristic acid, palmitic acid, stearic acid, arachidic acid, behenic acid, lignoceric acid, or cerotic acid), unsaturated (e.g., myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, or docosahexaenoic acid) or a combination thereof. Unsaturated fatty acids can be cis or trans fatty acids. In some embodiments, unsaturated fatty acids used in the complexes of the disclosure are cis fatty acids.

### 5.1.4.4. Apolar Molecules and Sterols Attached to a Sugar

The complexes can contain one or more amphipathic molecules that comprise an apolar molecule or moiety (e.g., a hydrocarbon chain, an acyl or diacyl chain) or a sterol (e.g., cholesterol) attached to a sugar (e.g., a monosaccharide such as glucose or galactose, or a disaccharide such as maltose or trehalose). The sugar can be a modified sugar or a substituted sugar. Exemplary amphipathic molecules comprising an apolar molecule attached to a sugar include dodecan-2-yloxy-β-D-maltoside, tridecan-3-yloxy-β-D-maltoside, tridecan-2-yloxy-β-D-maltoside, n-dodecyl-β-D-maltoside (DDM), n-octyl-β-D-glucoside, n-nonyl-β-D-glucoside, n-decyl-β-D-maltoside, n-dodecyl-β-D-maltopyranoside, 4-n-Dodecyl-α,α-trehalose, 6-n-dodecyl-α,α-trehalose, and 3-n-dodecyl-α,α-trehalose.

In some embodiments, the apolar moiety is an acyl or a diacyl chain.

In some embodiments, the sugar is a modified sugar or a substituted sugar.

### 5.1.5. Formulations

Lipid binding protein-based complexes can be formulated for the intended route of administration, for example according to techniques known in the art (e.g., as described in Allen et al., eds., 2012, Remington: The Science and Practice of Pharmacy, 22nd Edition, Pharmaceutical Press, London, UK).

CER-001 intended for administration by infusion can be formulated in a phosphate buffer with sucrose and mannitol excipients, for example as described in WO 2012/109162.

### 5.2. Subject Populations

Subjects who can be treated according to the methods described herein are preferably mammals, most preferably human.

In some aspects, the subject has or at risk of developing leukocytosis. Leukocytosis can be due to various conditions such as infections, inflammatory processes, and primary bone marrow disorders (e.g., acute leukemias, chronic leukemias and myeloproliferative disorders). Physical stress, for example from seizures, anesthesia or overexertion, and emotional stress can also elevate white blood cell counts. Leukocytosis can also be caused by medications, for example corticosteroids, lithium and beta agonists. Increased eosinophil or basophil counts, resulting from a variety of infections, allergic reactions and other causes, can also sometimes lead to leukocytosis.

In some embodiments, a subject with leukocytosis has a white blood cell count greater than 11×10⁹ per L and/or below 100×10⁹ per L. In some embodiments, the subject has a white blood cell count between 11×10⁹ per L and 50×10⁹ per L, between 11×10⁹ per L and 25×10⁹ per L, between 25×10⁹ per L and 50×10⁹ per L, or between 50×10⁹ per L and 100×10⁹ per L.

In some embodiments, the subject has a white blood cell count more than 11×10⁹ per L, more than 12×10⁹ per L, more than 15×10⁹ per L, or more than 20×10⁹ per L. Alternatively or in addition, in some embodiments, the subject has a white blood cell count less than 100×10⁹ per L, less than 50×10⁹ per L, less than 25×10⁹ per L, or less than 15×10⁹ per L.

In some embodiments, the subject has a white blood cell count between 11×10⁹ per L and 15×10⁹ per L, or between 15×10⁹ per L and 20×10⁹ per L

Subjects can have one or more symptoms associated with leukocytosis. Exemplary symptoms associated with leukocytosis include fever, bleeding or bruising, sweating, pain or tingling in the legs, arms, or abdomen, a vision problem (e.g., blurred vision, double vision, blind spots, cloudy vision, dimmed vision, or a combination thereof), unclear thinking, loss of appetite, or trouble breathing (e.g., shortness of breath, below normal blood oxygen levels).

In some embodiments, a subject has or is at risk of developing leukocytosis due to inflammation, an infection, a white blood cell disorder, physical stress, emotional stress, medication, or an allergic reaction.

In some embodiments, a subject having or at risk of developing leukocytosis has an infection, for example a bacterial, fungal, parasitic, or viral infection (e.g., a coronavirus infection such as COVID-19 or influenza).

In some embodiments, a subject having or at risk of developing leukocytosis has diabetes.

In some embodiments, a subject having or at risk of developing leukocytosis has received or is receiving a medication having leukocytosis as a side effect, for example a steroid, corticosteroid, lithium, or a beta agonist.

In some embodiments, a subject having or at risk of developing leukocytosis has a white blood cell disorder.

In some embodiments, the while blood cell disorder is a primary bone marrow disorder (e.g., an acute leukemia, chronic leukemia, or myeloproliferative disorder).

In some embodiments, the while blood cell disorder is neutrophilia (for example idiopathic neutrophilia, which in some embodiments is chronic idiopathic neutrophilia).

In some embodiments, the while blood cell disorder is hemolytic anemia.

In some embodiments, the while blood cell disorder is thrombocytopenia (for example idiopathic thrombocytopenia or essential thrombocytopenia).

In some embodiments, the while blood cell disorder is a cancer (for example a bone cancer or blood cancer).

In some embodiments, the while blood cell disorder is a myeloproliferative disorder.

In some embodiments, the while blood cell disorder is a blood cancer.

In some embodiments, the while blood cell disorder is lymphoma.

In some embodiments, the while blood cell disorder is leukemia (for example lymphocytic leukemia, chronic eosinophilic leukemia, chronic myelogenous leukemia, or chronic neutrophilic leukemia).

In some embodiments, the while blood cell disorder is polycythemia vera.

In some embodiments, the while blood cell disorder is myelofibrosis, for example primary myelofibrosis.

In some embodiments of the methods of treating a subject having or at risk of developing leukocytosis and/or methods of treating a subject having one or more symptoms associated with leukocytosis, administration of the lipid binding protein-based complex is effective to reduce the subject's white blood cell count and/or ameliorate one or more symptoms associated with leukocytosis.

In some aspects, the subject has endothelial dysfunction (for example a subject experiencing acute coronary syndrome or stroke or who has experienced acute coronary syndrome or stroke). Examples of acute coronary syndrome include myocardial infarction (for example ST-elevation myocardial infarction, non-ST elevation myocardial infarction) and unstable angina.

In some embodiments, the subject is experiencing acute coronary syndrome. In other embodiments, the subject has experienced acute coronary syndrome. In other embodiments, the subject is experiencing a stroke. In other embodiments, the subject has experienced a stroke.

In some aspects, the subject having or at risk of developing carditis (for example myocarditis and/or pericarditis). In some embodiments, the subject is male. In some embodiments, the subject is less than 60 years old, less than 50 years old, less than 40 years old, less than 30 years old, less than 20 years old and/or at least 5 years old, at least 8 years old, at least 10 years old, at least 12 years old, at least 15 years old, or at least 18 years old.

In some embodiments, the subject having or at risk of developing carditis has an infection, for example a viral infection or a bacterial infection. Exemplary viral infections include influenza and coronavirus infections. In some embodiments, the subject has a COVID-19 infection. COVID-19 vaccines, for example, mRNA COVID-19 vaccines, have been associated with carditis, especially in young males. Thus, in some embodiments, the subject at risk of developing carditis is a young male (for example, at least 5, 8, 10, 12, 15, or 18 years of age and less than 30 or 20 years of age). In some embodiments, the subject has received a vaccine (e.g., a COVID-19 vaccine) prior to (e.g., 1 to 10 days) administration of the lipid binding protein-based complex. In other embodiments, the subject receives a vaccine (e.g., a COVID-19 vaccine) after (e.g., 1 to 10 days) administration of the lipid binding protein-based complex.

In other embodiments, the subject receives a vaccine (e.g., a COVID-19 vaccine) concurrently with administration of the lipid binding protein-based complex (e.g., on the same day).

In some embodiments of the methods of the disclosure, the subject has a SOFA score of 1 to 4 before treatment with a lipid binding protein-based complex, e.g., a score of 1, 2, 3, or 4 (see, Vincent et al. 1996, Intensive Care Med, 22:707-710).

In some embodiments of the methods of the disclosure, the subject has an endotoxin activity level as measured by the Endotoxin Activity Assay (EEA™) (Spectral Medical) of >0.6 prior to administration of the lipid binding protein-based complex (see, Marshall et al., 2004, J Infect Dis. 190(3):527-34).

In another aspect, the subject is a subject in need of a reduction in serum levels of one or more inflammatory markers, for example a subject with elevated levels of the one or more inflammatory markers compared to normal levels. Exemplary inflammatory cytokines include interleukin 6 (IL-6), C-reactive protein, D-dimer, ferritin, interleukin 8 (IL-8), granulocyte-macrophage colony stimulating factor (GM-CSF), monocyte chemoattractant protein (MCP) 1, and tumor necrosis factor α (TNFα). In some embodiments, the one or more cytokines comprise IL-6. In some embodiments, the one or more cytokines comprise a combination of the foregoing, for example, 2, 3, 4, 5, 6, 7, or all 8 of interleukin 6 (IL-6), C-reactive protein, D-dimer, ferritin, interleukin 8 (IL-8), granulocyte-macrophage colony stimulating factor (GM-CSF), monocyte chemoattractant protein (MCP) 1, and tumor necrosis factor α (TNFα).

### 5.3. Dosing Regimens

The methods of the disclosure typically entail multiple administrations of a lipid binding protein-based complex (e.g., CER-001), e.g., two to 20 individual doses (e.g., 2 to 16, 2 to 12, 2 to 10, 2 to 8, 2 to 6, 2 to 4, 4 to 20, 4 to 16, 4 to 10, 4 to 6, 6 to 20, 6 to 16, 6 to 10, 6 to 8, 8 to 20, 8 to 16, 8 to 10, 10 to 20, 10 to 16, or 16 to 20 individual doses). In some embodiments, an administration regimen can include four or more doses of a lipid binding protein-based complex (e.g., CER-001), e.g., five, six, seven, eight, nine, ten, eleven, twelve, or more than twelve doses.

In some embodiments, the lipid binding protein-based complex is administered according to an induction and, optionally, a consolidation regimen as described in Sections 5.3.1 and 5.3.2, respectively. In some embodiments, the lipid binding protein-based complex can be administered in a single phase, e.g., according to an administration regimen described in this Section. In some embodiments, the subject is not treated with the lipid binding protein-based complex according to a maintenance regimen, e.g., a regimen comprising long-term (e.g., one month or longer) administration of the lipid binding protein-based complex.

The lipid binding protein-based complex (e.g., CER-001) administration regimens of the disclosure can last up to one week, one week, or more than one week (e.g., two weeks).

For example, a lipid binding protein-based complex (e.g., CER-001) administration regimen can comprise administering:
five doses of CER-001 over one week;
six doses of CER-001 over one week;
seven doses of CER-001 over one week;
ten doses of CER-001 over two weeks;
twelve doses of CER-001 over two weeks;
fourteen doses of CER-001 over two weeks.

In an embodiment, the methods of the disclosure comprise administering seven doses of CER-001 over one week, e.g., on days 1, 2, 3, 4, 5, 6, and 7.

In some embodiments of the methods of the disclosure, a lipid binding protein-based complex (e.g., CER-001) is administered daily, e.g., daily for at least 5 days, at least 6 days, at least 7 days, or more than 7 days (e.g., daily for up to one week or daily for up to two weeks). In some embodiments of the methods of the disclosure, a lipid binding protein-based complex (e.g., CER-001) multiple doses approximately 12 hours apart (e.g., four to six doses administered approximately 12 hours apart). In other embodiments, a lipid binding protein-based complex (e.g., CER-001) is administered less frequently, e.g., every other day, two times per week, three times per week, or once a week.

In practice, an administration window can be provided, for example, to accommodate slight variations to a multi-dosing per week dosing schedule. For example, a window of ±2 days or ±1 day around the dosage date can be used.

A lipid binding protein-based complex (e.g., CER-001) can be administered in the methods of the disclosure for a pre-determined period of time, e.g., for one week. Alternatively, administration of a lipid binding protein-based complex (e.g., CER-001) can be continued until one or more symptoms of condition are reduced or continued until the serum levels of one or more inflammatory markers are reduced, for example reduced to a normal level or reduced relative to a baseline value for the subject, e.g., a baseline value measured prior to the start of lipid binding protein-based complex (e.g., CER-001) therapy. Reference or “normal” levels of various inflammatory markers are known in the art. For example, the Mayo Clinic Laboratories test catalog (www.mayocliniclabs.com/test-catalog) provides the following reference values: IL-6: ≤1.8 pg/mL; C-reactive protein: ≤8.0 mg/mL; D-dimer: ≤500 ng/mL Fibrinogen Equivalent Units (FEU); ferritin: 24-336 mcg/L (males), 11-307 mcg/L (females); IL-8<57.8 pg/mL; TNF-α <5.6 pg/mL.

The methods of the disclosure (e.g., methods for treating a condition or symptom described herein) typically comprise administering a high dose of a lipid binding protein-based complex (e.g., CER-001). The high dose can be the aggregate of multiple individual doses (e.g., two, three, four, five, six, seven, eight, nine, 10, or more than 10 individual doses), for example administered over multiple days (e.g., a period of three days, four days, five days, six days, seven days, eight days, nine days, 10 days, eleven days, 12 days, 13 days, 14 days or 15 days). The individual doses of a high dose are in some embodiments administered daily, twice daily (e.g., approximately 12 hours apart), or two to three days apart.

In some embodiments, a high dose is effective to ameliorate one or more symptoms associated with leukocytosis.

In some embodiments, the high dose is an amount effective to increase the subject's HDL and/or ApoA-I blood levels and/or improve the subject's vascular endothelial function, e.g., measured by circulating vascular cell adhesion molecule 1 (VCAM-1) and/or intercellular adhesion molecule 1 (ICAM-1) levels. In some embodiments, the high dose or an individual dose is an amount which increases the subject's HDL and/or ApoA-I levels by at least 25%, at least 30%, or at least 35% 2 to 4 hours after administration.

In embodiments, a high dose is effective to reduce the subject's circulating VCAM-1 by at least 100 ng/mL, at least 200 ng/mL, at least 300 ng/mL, and/or by up to 400 ng/mL within two days of the first administration of the lipid binding protein-based complex. In embodiments, a high dose is effective to reduce the subject's circulating VCAM-1 by at least 100 ng/mL, at least 200 ng/mL, at least 300 ng/mL, and/or by up to 400 ng/mL within five days of the first administration of the lipid binding protein-based complex.

In embodiments, a high dose is effective to reduce the subject's circulating ICAM-1 by at least 50 ng/mL, at least 75 ng/mL, at least 100 ng/mL and/or by up to 100 ng/mL or up to 125 ng/mL within two days of the first administration of the lipid binding protein-based complex. In embodiments, a high dose is effective to reduce the subject's circulating ICAM-1 by at least 50 ng/mL, at least 75 ng/mL, at least 100 ng/mL and/or by up to 125 ng/mL within five days of the first administration of the lipid binding protein-based complex.

In some embodiments, the high dose is an amount effective to reduce serum levels of one or more inflammatory markers, for example, one or more of IL-6, C-reactive protein, D-dimer, ferritin, IL-8, GM-CSF, MCP1, and TNF-α. In some embodiments, the serum levels of the one or more inflammatory markers are reduced from an elevated range to a normal range, and/or reduced by at least 20%, at least 40%, or at least 60%.

In some embodiments, a high dose is effective to reduce serum levels of IL-6 by at least 20 μg/mL, at least 30 μg/mL, or at least 40 μg/mL and/or by up to 40 μg/mL within two days of the first administration of the lipid binding protein-based complex. In some embodiments, a high dose is effective to reduce serum levels of IL-6 by at least 20 μg/mL, at least 30 μg/mL, or at least 40 μg/mL and/or by up to 50 μg/mL or up to 60 μg/mL within five days of the first administration of the lipid binding protein-based complex.

In some embodiments, a high dose is effective to reduce serum levels of C-reactive protein.

In some embodiments, a high dose is effective to reduce serum levels of D-dimer.

In some embodiments, a high dose is effective to reduce serum levels of ferritin. In some embodiments, the high dose is effective to reduce serum levels of ferritin by at least 200 ng/mL, at least 300 ng/mL, or at least 400 ng/mL and/or by up to 700 ng/mL within two days of the first administration of the lipid binding protein-based complex. In some embodiments, the high dose is effective to reduce serum levels of ferritin by at least 200 ng/mL, at least 300 ng/mL, or at least 400 ng/mL and/or by up to 700 ng/mL within five days of the first administration of the lipid binding protein-based complex.

In some embodiments, a high dose is effective to reduce serum levels of interleukin 8 (IL-8). In some embodiments, a high dose is effective to reduce serum levels of IL-8 by at least 100 μg/mL, at least 150 μg/mL and/or by up to 300 μg/mL within two days of the first administration of the lipid binding protein-based complex. In some embodiments, a high dose is effective to reduce serum levels of IL-8 by at least 100 μg/mL, at least 150 μg/mL and/or by up to 300 μg/mL within five days of the first administration of the lipid binding protein-based complex.

In some embodiments, the high dose is effective to reduce serum levels of monocytechemoattractant protein (MCP) 1 and/or tumor necrosis factor α (TNF-α) and/or KIM-1.

In some embodiments, a high dose is effective to reduce the subject's white blood cell count. In some embodiments, a high dose is effective to reduce the subject's white blood cell count by at least 2000 WBCs/mL, at least 3000 WBCs/mL, at least 4000 WBCs/mL, at least 5000 WBCs/mL and/or by up to 8000 WBCs/mL within two days of the first administration of the lipid binding protein-based complex. In some embodiments, a high dose is effective to reduce the subject's white blood cell count by at least 2000 WBCs/mL, at least 3000 WBCs/mL, at least 4000 WBCs/mL, at least 5000 WBCs/mL and/or by up to 6000 WBCs/mL within five days of the first administration of the lipid binding protein-based complex.

In some embodiments, the high dose is effective to transiently increase serum triglyceride levels, for example for up to 9 days.

In some embodiments, a high dose is a dose effective to prevent carditis in a subject at risk for carditis or reduce the severity of the carditis in a subject having carditis or at risk of carditis.

The dose of a lipid binding protein-based complex (e.g., CER-001) administered to a subject (e.g., an individual dose which when aggregated with one or more other individual doses forms a high dose) can in some embodiments range from 4 to 40 mg/kg (e.g., 10 to 40 mg/kg) on a protein weight basis (e.g., 5, 10, 15, 20, 25, 30, 35, or 40 mg/kg or any range bounded by any two of the foregoing values, e.g., 10 to 20 mg/kg, 15 to 25 mg/kg, 20 to 40 mg/kg, 25 to 35 mg/kg, or 30 to 40 mg/kg). As used herein, the expression “protein weight basis” means that a dose of a lipid binding protein-based complex (e.g., CER-001) to be administered to a subject is calculated based upon the amount of ApoA-I in the lipid binding protein-based complex (e.g., CER-001) to be administered and the weight of the subject. For example, a subject who weighs 70 kg and is to receive a 20 mg/kg dose of CER-001 would receive an amount of CER-001 that provides 1400 mg of ApoA-I (70 kg×20 mg/kg).

In yet other aspects, a lipid binding protein-based complex (e.g., CER-001) can be administered on a unit dosage basis. The unit dosage used in the methods of the disclosure can in some embodiments vary from 300 mg to 4000 mg (e.g., 600 mg to 4000 mg) per administration (on a protein weight basis).

In particular embodiments, the dosage of a lipid binding protein-based complex (e.g., CER-001) is 600 mg to 3000 mg, 800 mg to 3000 mg, 1000 mg to 2400 mg, or 1000 mg to 2000 mg per administration (on a protein weight basis).

In some aspects, a high dose of a lipid binding protein-based complex (e.g., CER-001), e.g., the aggregate of multiple individual doses, is 600 mg to 40 g (on a protein weight basis). In particular embodiments, a high dose is 3 g to 35 g or 5 g to 30 g (on a protein weight basis).

A lipid binding protein-based complex (e.g., CER-001) is preferably administered as an IV infusion. For example, a stock solution of CER-001 can be diluted in normal saline such as physiological saline (0.9% NaCl) to a total volume between 125 and 250 ml. In some embodiments, subjects weighing less than 80 kg will have a total volume of 125 ml whereas subjects weighing at least 80 kg will have a total volume of 250 ml. In some embodiments, doses of CER-001 are administered in a total volume of 250 ml. A lipid binding protein-based complex (e.g., CER-001) may be administered over a period ranging from one-hour to 24-hours. Depending on the needs of the subject, administration can be by slow infusion with a duration of more than one hour (e.g., up to 2 hours or up to 24 hours), by rapid infusion of one hour or less, or by a single bolus injection. In an embodiment, a lipid binding protein-based complex (e.g., CER-001) is administered over a one-hour period, e.g., using an infusion pump at a fixed rate of 125 ml/hr or 250 ml/hr. In an embodiment, a dose of a lipid binding protein-based complex (e.g., CER-001) is administered as an infusion over a 24-hour period.

### 5.3.1. Induction Regimen

In one embodiment, induction regimens suitable for use in the methods of the disclosure entail administering multiple doses of a lipid binding protein-based complex (e.g., CER-001) over multiple consecutive days, e.g., three consecutive days.

In some embodiments, induction regimens suitable for use in the methods of the disclosure entail twice daily administration of a lipid binding protein-based complex (e.g., CER-001) such as twice daily administration on multiple consecutive days. Twice daily administration can comprise, for example, two doses approximately 12 hours apart or a morning dose and an evening dose (which may be more or less than 12 hours apart).

In an embodiment, the induction regimen comprises two doses of a lipid binding protein-based complex (e.g., CER-001) per day for 3 consecutive days.

A therapeutic dose of a lipid binding protein-based complex (e.g., CER-001) administered by infusion in the induction regimen can range from 4 to 40 mg/kg (e.g., 4 to 30 mg/kg) on a protein weight basis (e.g., 4, 5, 6, 7, 8, 9, 10, 12 15, 20, 25, 30 or 40 mg/kg, or any range bounded by any two of the foregoing values, e.g., 5 to 15 mg/kg, 10 to 20 mg/kg, or 15 to 25 mg/kg). In some embodiments, the dose of a lipid binding protein-based complex (e.g., CER-001) used in the induction regimen is 5 mg/kg. In some embodiments, the dose of a lipid binding protein-based complex (e.g., CER-001) used in the induction regimen is 10 mg/kg. In some embodiments, the dose of a lipid binding protein-based complex (e.g., CER-001) used in the induction regimen is 15 mg/kg. In some embodiments, the dose of a lipid binding protein-based complex (e.g., CER-001) used in the induction regimen is 20 mg/kg. In some embodiments, the induction regimen comprises six doses of a lipid binding protein-based complex (e.g., CER-001) administered over three days at a dose of 5 mg/kg, 10 mg/kg, 15 mg/kg or 20 mg/kg.

In yet other aspects, a lipid binding protein-based complex (e.g., CER-001) can be administered on a unit dosage basis. The unit dosage used in the induction phase can vary from 300 mg to 4000 mg (e.g., 300 mg to 3000 mg) (on a protein weight basis) per administration by infusion.

In particular embodiments, the dosage of a lipid binding protein-based complex (e.g., CER-001) used during the induction phase is 300 mg to 1500 mg, 400 mg to 1500 mg, 500 mg to 1200 mg, or 500 mg to 1000 mg (on a protein weight basis) per administration by infusion.

### 5.3.2. Consolidation Regimen

Consolidation regimens suitable for use in the methods of the disclosure entail administering one dose or multiple doses of a lipid binding protein-based complex (e.g., CER-001) following an induction regimen.

In one embodiment, the consolidation regimen comprises administering two doses of a lipid binding protein-based complex (e.g., CER-001). For example, the two doses can be administered approximately 12 hours apart, or administered as a morning dose and an evening dose (which may be more or less than 12 hours apart).

The dose(s) of a lipid binding protein-based complex (e.g., CER-001) in a consolidation regimen can in some embodiments be administered on day 6 of a dosing regimen that begins with an induction regimen on day 1. The dose(s) of a lipid binding protein-based complex (e.g., CER-001) in a consolidation regimen can in some embodiments be administered on day 4 of a dosing regimen that begins with an induction regimen on day 1. The dose(s) of a lipid binding protein-based complex (e.g., CER-001) in a consolidation regimen can in some embodiments be administered on day 5 of a dosing regimen that begins with an induction regimen on day 1. The dose(s) of a lipid binding protein-based complex (e.g., CER-001) in a consolidation regimen can in some embodiments be administered on day 7 of a dosing regimen that begins with an induction regimen on day 1.

A therapeutic dose of a lipid binding protein-based complex (e.g., CER-001) administered by infusion in the consolidation regimen can range from 4 mg/kg to 40 mg/kg (e.g., 4 to 30 mg/kg) on a protein weight basis (e.g., 4, 5, 6, 7, 8, 9, 10, 12, 15, 20, 25, 30, or 40 mg/kg, or any range bounded by any two of the foregoing values, e.g., 5 to 15 mg/kg, 10 to 20 mg/kg, or 15 to 25 mg/kg). In some embodiments, the dose of a lipid binding protein-based complex (e.g., CER-001) used in the consolidation regimen is 5 mg/kg. In some embodiments, the dose of a lipid binding protein-based complex (e.g., CER-001) used in the consolidation regimen is 10 mg/kg. In some embodiments, the dose of a lipid binding protein-based complex (e.g., CER-001) in the consolidation regimen is 15 mg/kg. In some embodiments, the dose of a lipid binding protein-based complex (e.g., CER-001) used in the consolidation regimen is 20 mg/kg. In some embodiments, the consolidation regimen comprises two doses of a lipid binding protein-based complex (e.g., CER-001) administered on one day at a dose of 5 mg/kg, 10 mg/kg, 15 mg/kg or 20 mg/kg.

In yet other aspects, a lipid binding protein-based complex (e.g., CER-001) can be administered on a unit dosage basis. The unit dosage used in the consolidation phase can vary from 300 mg to 4000 mg (e.g., 300 mg to 3000 mg) (on a protein weight basis) per administration by infusion.

In particular embodiments, the dosage of a lipid binding protein-based complex (e.g., CER-001) used during the consolidation phase is 300 mg to 1500 mg, 400 mg to 1500 mg, 500 mg to 1200 mg, or 500 mg to 1000 mg (on a protein weight basis) per administration by infusion.

The lipid binding protein-based complex (e.g., CER-001) can be administered during the consolidation phase in the same manner as described in Section 5.3, e.g., as an IV infusion over a one-hour period.

### 5.4. Combination Therapies

A lipid binding protein-based complex (e.g., CER-001) can be administered to a subject as described herein as a monotherapy or a part of a combination therapy regimen. For example, a combination therapy may comprise a lipid binding protein-based complex (e.g., CER-001) in combination with a standard of care treatment for sepsis or other infection. See, e.g., Rhodes et al., 2017, Intensive Care Med 43:304-377; Dugar et al., 2020, Cleveland Clinic Journal of Medicine 87(1):53-64.

In some embodiments, the subject is treated with a lipid binding protein-based complex (e.g., CER-001) in combination with fluid replacement therapy. In some embodiments, the subject is treated with a lipid binding protein-based complex (e.g., CER-001) in combination with an antimicrobial. In some embodiments, the subject is treated with a lipid binding protein-based complex (e.g., CER-001) in combination with an antibiotic (e.g., ceftriaxone, meropenem, ceftazidime, cefotaxime, cefepime, piperacillin and tazobactam, ampicillin and sulbactam, imipenem and cilastatin, levofloxacin, or clindamycin). In some embodiments, the subject is treated with a lipid binding protein-based complex (e.g., CER-001) in combination with an antiviral. In some embodiments, the subject is treated with a lipid binding protein-based complex (e.g., CER-001) in combination with a medication that raises blood pressure (e.g., norepinephrine or epinephrine).

A combination therapy regimen can in some embodiments comprise one or more anti-IL-6 agents and/or one or more other agents for treating CRS such as corticosteroids (e.g., methylprednisolone and/or dexamethasone). Exemplary anti-IL6 agents include tocilizumab, siltuximab, olokizumab, elsilimomab, BMS-945429, sirukumab, levilimab, and CPSI-2364. In some embodiments, a lipid binding protein-based complex (e.g., CER-001) is administered in combination with tocilizumab. Subjects who have or have had a COVID-19 infection can be treated with a lipid binding protein-based complex (e.g., CER-001) in combination with one or more additional therapies such as antibodies from recovered COVID-19 patients, antibodies against the spike protein of COVID-19, one or more antiviral agents (e.g., lopinavir, remdesivir, danoprevir, galidesivir, darunavir, ritonavir), chloroquine, hydroxychloroquine, azithromycin, an interferon (e.g., an interferon alpha or an interferon beta, each of which can be pegylated), or a combination thereof.

In certain embodiments, an antihistamine (e.g., diphenhydramine, cetirizine, fexofenadine, or loratadine) can be administered before administration of a lipid binding protein-based complex (e.g., CER-001). The antihistamine can reduce the likelihood of allergic reactions.

### 6. EXAMPLES

### 6.1. Example 1: CER-001 Therapy in a Swine Model of LPS-Induced AKI

The ability of CER-001 to mitigate sepsis-related AKI was evaluated in a lipopolysaccharide (LPS)-induced swine model of AKI.

### 6.1.1. Materials and Methods

Pigs were randomized into three groups: LPS (endotoxemic pigs, n=3), single dose CER-001 treated pigs (endotoxemic pigs treated with a single dose of CER-001 at 20 mg/kg; n=3), and multiple dose CER-001 treated pigs (endotoxemic pigs treated with two doses of CER-001 at 20 mg/kg; n=3).

Sepsis was induced in the pigs by intravenous infusion of a saline solution containing 300 μg/kg of LPS at TO. Single dose CER-001 treated pigs and CER-001 multiple dose treated pigs received a 20 mg/kg dose of CER-001 at TO. CER-001 multiple dose treated pigs received a second 20 mg/kg dose of CER-001 three hours later (T3). Serum IL-6, LPS, MCP-1, sVCAM-1 and sICAM-1 levels were monitored overtime. Renal tissue damage and fibrosis were assessed at the end of the study period.

### 6.1.2. Results

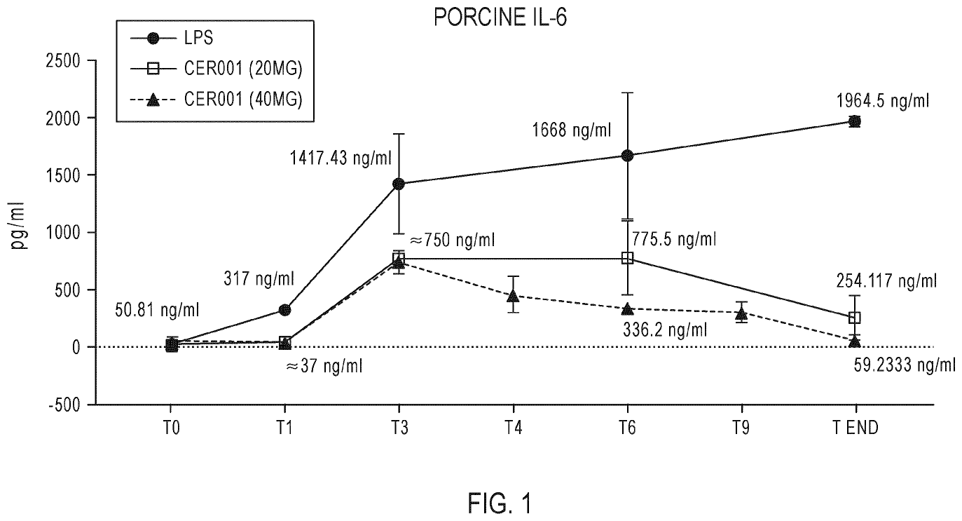

An increased survival rate was observed in both CER-001 treated groups compared to LPS group (data not shown). LPS injection led to a time-dependent increase of IL-6 in endotoxemic animals (FIG. 1) compared to the basal condition (TO). CER-001 treatment was able to reverse LPS effects, as shown by reduced IL-6 levels (FIG. 1, “20 MG” and “40MG”). The second infusion of CER-001 three hours from the first dose (T3) strongly reduced IL-6 serum levels to basal level by the end of the study (T end) (FIG. 1, “40MG”). Similarly, high levels of MCP-1 in endotoxemic pigs were observed relative to the basal condition, while MCP-1 levels were lower in the pigs treated with CER-001 (data not shown).

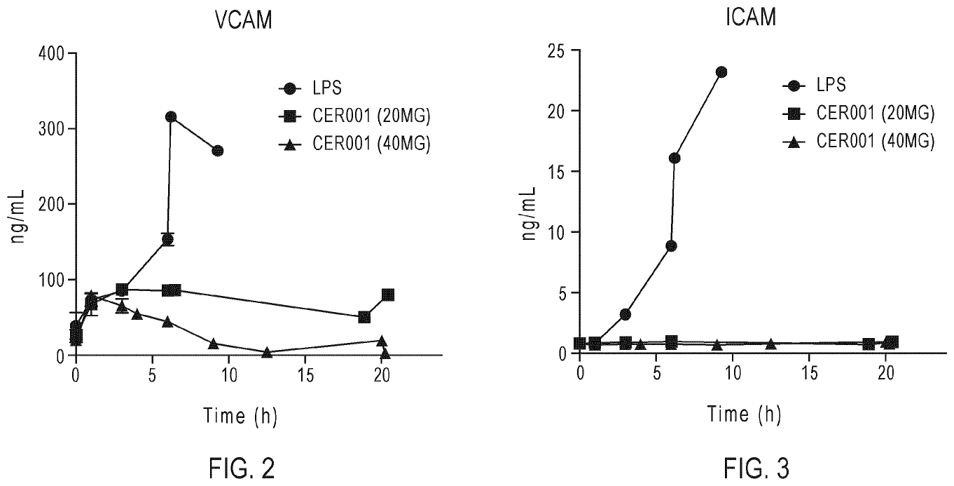

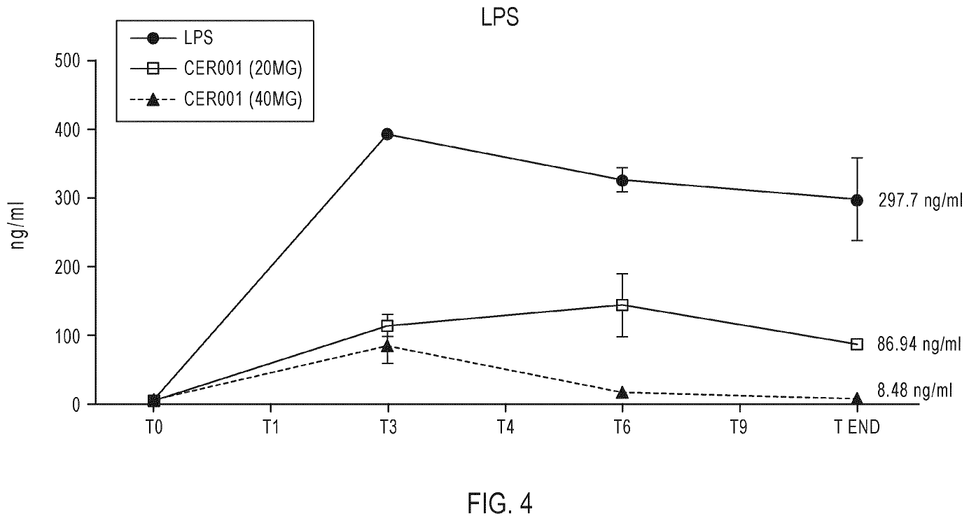

Endothelial dysfunction was evaluated by measuring sVCAM-1 and sICAM-1 serum levels. Time-dependent increases of sVCAM-1 and sICAM-1 were observed in endotoxemic animals, while CER-001 treatment strongly decreased sVCAM-1 and sICAM-1 levels in both treated groups (FIG. 2 and FIG. 3, respectively). In line with IL-6 results, the infusion of two doses of CER-001 (FIG. 2, “40 MG”) was more efficient in decreasing sVCAM-1 to basal levels. LPS levels were strongly reduced in CER-001 treated animals (FIG. 4, “20MG” and “40 MG”) and the effects were more evident after the second infusion of CER-001 (FIG. 4, “40 MG”).

The endotoxemic renal biopsies presented tubular vacuolization, epithelial flattening, and some apoptotic tubular cells. CER-001 treatment significantly decreased inflammatory processes and tubular damage. In endotoxemic animals, Masson's trichrome staining revealed extensive collagen deposition at the interstitial level. In both CER-001 treated groups, there were significantly fewer collagen deposits in renal parenchymal compared to the LPS group.

This preclinical data indicates that CER-001 treatment reduces systemic inflammation and endothelial dysfunction, thereby limiting renal damage in the LPS-induced swine model of AKI.

### 6.2. Example 2: Randomized Pilot Study Comparing Short-Term CER-001 Infusions at Different Doses to Prevent Sepsis-Induced Acute Kidney Injury

Currently, there are no approved treatments for sepsis-related AKI. Considering that the inflammatory response to endotoxemia is a major cause for hemodynamic destabilization and progression to AKI in septic patients, the main objective of the study is to investigate whether the use of CER-001 at different doses in combination with standard of care (SOC) treatment is safe and effective, providing a new strategy to treat septic patients, reducing the inflammatory response and preventing the progression to AKI. Without being bound by theory, the anticipated mechanism of action is two-fold, comprising both the binding of endotoxin by CER-001 and a direct anti-inflammatory effect of CER-001.

### 6.2.1. Study Protocol

Study population: This is a single-center, randomized, dose-ranging (phase II) study including patients with sepsis due to intra-abdominal cavity infection or urosepsis, admitted at the Intensive Care Unit (ICU) of the participating center. The investigators ensure that all patients meeting the following inclusion and exclusion criteria are offered enrollment in the study.

### Inclusion Criteria:

Male or non-pregnant female adult ≥18 years of age at time of enrollment;
Meets Sepsis 3 criteria, defined as an acute increase of at least 2 points in SOFA Score relative to the SOFA score upon admission;
Endotoxin level (measured by Endotoxin Activity Assay (EEA™); Spectral Medical) >0.6 (see, Marshall et al., 2004, J Infect Dis. 190(3):527-34);
Signed and dated informed consent by the patient itself or by a legal representative.

### Exclusion Criteria:

Patients weighing more than 100 kg;
Alanine transaminase/aspartate transaminase (ALT/AST) >5 times the upper limit of normal;
Stage 4 severe chronic kidney disease or requiring dialysis (i.e. estimated glomerular filtration rate (eGFR)<30 ml/min/1.73 m²);
Leukocytes<2.0×10\hat{ }9;
Pregnancy or breast feeding;
Undergone organ transplantation during the past one year;
Anticipated transfer to another hospital, which is not a study site within 72 hours;
Terminally ill, including metastases or hematological malignancy, with a life expectancy less than 30 days (as assessed by the attending physician) or have been classified as “Do Not Resuscitate”;
Previous history of end stage chronic organ failure(s);
Diagnosed with HIV;
Uncontrolled hemorrhage within the last 24 h;
Patients who have used an investigational drug or device within 30 days of the first dose of CER-001.

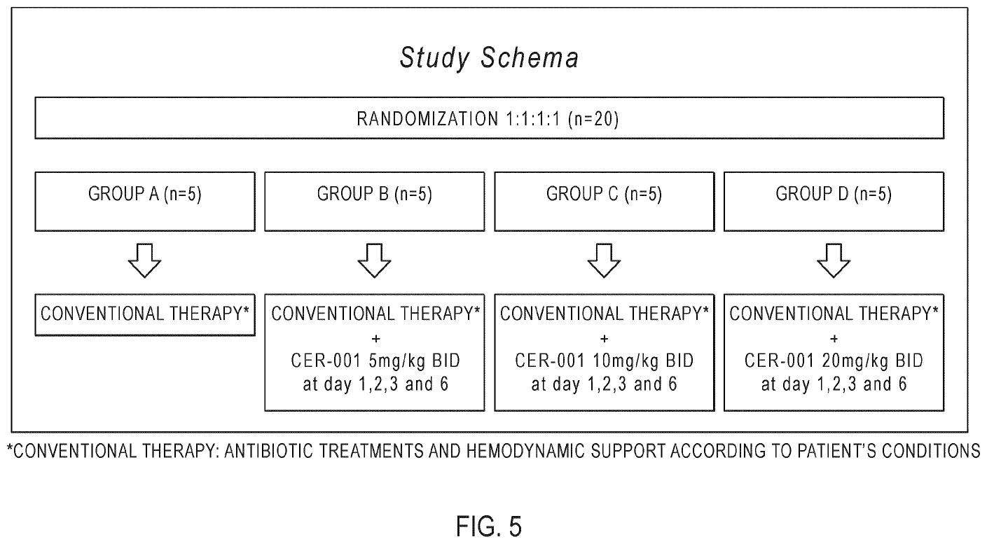

Number of subjects: Twenty subjects are enrolled and randomized (1:1:1:1) into four experimental groups: Group A patients continue to receive conventional therapy, Group B: patients add CER-001 5 mg/kg BID for 3 days to conventional therapy, followed by 5 mg/kg BID on Day 6; Group C: patients add CER-001 10 mg/kg BID for 3 days to conventional therapy, followed by 10 mg/kg BID on Day 6; Group D patients add CER-001 20 mg/kg BID for 3 days to conventional therapy, followed by 20 mg/kg BID on Day 6 (FIG. 5).

Duration of study: This study is completed in 24 weeks (6 months). The enrolment period is approximately 20 weeks (5 months) from the first subject enrolled. The end of the study is the last visit of the last subject.

Primary endpoint: The primary end-point of the study is to define the safety and the optimal dose of CER-001 in combination with standard of care in patients with sepsis sustained by Gram negative bacteria.

Secondary endpoint: Secondary end-points are:
Change in endotoxin and IL-6 levels from baseline to Day 3, Day 6 and Day 9.
Baseline is defined as the last measurements taken prior to dosing on Day 1.
Change in the SOFA score (Vincent et al. 1996, Intensive Care Med, 22:707-710) from baseline to Day 3, Day 6 and Day 9.
Changes to the key inflammatory markers (CRP, D-dimer, Ferritin, IL-8, GM-CSF, MCP 1 and TNF-α) from baseline to Day 3, Day 6 and Day 9.
Changes in AKI biomarkers and onset of AKI according to KDIGO criteria (Kidney Disease Improving Global Outcomes. KDIGO Clinical Practice Guideline for Acute Kidney Injury. Kidney International Supplements 2012; 2: 1-138)
Mortality at Day 30
An independent medical expert review outcome data during the trial.

Intervention/exposure: Twenty patients meeting the eligibility criteria, who sign and date an ethical committee (EC)-approved informed consent form, are randomized and assigned (1:1:1:1) ratio to conventional therapy (Group A), low dose CER-001 (Group B) or medium dose CER-001 (Group C) or high dose CER-001 (Group D). Conventional therapy is modulated according to the clinical conditions. All non-experimental treatments are allowed to be administered concomitantly during the patient's participation in this study: any medication the patient takes, other than study drugs specified per protocol, is considered a concomitant medication and is recorded in the study records.

Each patient is identified at the screening by a patient number. Once assigned to a patient, the patient number is not reused. The randomization list and the allocation assignment sequence is produced and the investigators that enroll do not have any participation in this task. The randomization list divided into blocks is adequately concealed to prevent attempts at subversion of randomization.

Treatment group: All patients receive conventional therapy. Treated groups receive an additional therapy with the study drugs. In particular:
Group A: Conventional therapy (i.e., antibiotic treatments and hemodynamic support according to patient's conditions).
Group B: Conventional therapy+CER-001 5 mg/kg BID for 3 consecutive days, followed by 5 mg/kg BID on Day 6.
Group C: Conventional therapy+CER-001 10 mg/kg BID for 3 consecutive days, followed by 10 mg/kg BID on Day 6.
Group D: Conventional therapy+CER-001 20 mg/kg BID for 3 consecutive days, followed by 20 mg/kg BID on Day 6.

Patients are pretreated with antihistamine prior to each CER-001 dose (e.g. dexchlorpheniramine 5 mg or hydroxyzine 100 mg) to avoid any potential infusion reactions. Patients may be interrupted or discontinued from study medication if any of the following occur:

Any drug-related adverse event or other reason which, in the Investigator's opinion, jeopardizes the patient's participation in the trial or the interpretation of trial data (e.g., severe inter-current illness requiring additional care measures or preventing further dosing); significant tolerability issues.

At the time of study medication interruption, the study site documents the reason for drug interruption. The patient continues to be followed clinically and all attempts are made to re-institute study medication within 2 days of the study drug interruption if not otherwise contraindicated.

Reasons for withdrawal from study drug may include, but are not limited to, the following:
Investigator's request, for safety reasons, such as severe adverse reactions;
Investigator's request, for other reasons, such as patient non-compliance;
Patient's request, for tolerability reasons;
Patient's request, for other reasons, such as withdrawal of informed consent.

Discontinuation of study drug alone does not constitute discontinuation or withdrawal from the study. Patients continue to be followed as though they had completed the treatment phase. Patients who prematurely discontinue study medication (e.g., prior to completion of the 3th dose) undergo end of study evaluations whenever possible.

Statistical analysis: Comparison between groups is performed using the appropriate statistical tests: dichotomous variables (baseline characteristics, mortality, development of AKI) are compared by the use of Chi-square or Fisher's exact test, continuous baseline characteristics by ANOVA or Kruskall-Wallis test, t Student or Mann-Whitney U test, as appropriate. Changes in inflammatory markers are compared between groups by ANOVA and are graphically represented. Proportion of patients of AKI and mortality rate are calculated for each group. All analyses is performed using SPSS 12.0 for Windows; p<0.05 is considered statistically significant.

Procedures: The following procedures are performed during the screening visit. Following randomization, subjects initiate treatment within 2 business days.
Informed consent
Medical history—includes: recording past and present illnesses and collection of the subjects demographic data (birth date, sex, and race).
Physical examination with a review of systems, height and weight, BMI and wait circumference
Vital signs (pulse, blood pressure, and oral, auricular, axillary, or core temperature).
Review of inclusion/exclusion criteria.
Adverse events are recorded starting from the time informed consent is obtained.
Prior medications are collected from 4 weeks before the first dose of test article. All current medications are recorded.
Complete blood count (CBC)—includes white blood cell count (WBC) with differential, platelet count, red blood cell count (RBC), haemoglobin (Hb), hematocrit (Hct).
Fasting chemistry panel/electrolytes: includes sodium, potassium, chloride, blood urea nitrogen (BUN; or urea), serum creatinine, calculated clearance creatinine (CKD-EPI), glucose, calcium, phosphorus, total protein, uric acid, AST, ALT, γ GT, ALP, total and direct bilirubine, albumin, total cholesterol, HDL, LDL, triglycerides, LDH, CPK,
ABG (for assessing respiratory and/or metabolic disorders)
ApoA-I (for pharmacokinetic and pharmacodynamic assessment)
Coagulation tests—includes prothrombin time (PT) (expressed as international normalized ratio [INR]), and partial thromboplastin time (PTT).
Urinalysis—includes specific gravity, pH, assessment of protein/albumin, glucose, ketones, and haemoglobin/blood.
Microalbumunuria and Proteinuria g/24 h
Serum or urine pregnancy test (for women of childbearing potential) within 7 days before randomization.
Pharmacokinetic and pharmacodynamic assessment includes apoA-I and total cholesterol levels.
Endotoxin levels are measured using the EAA™ kit. AKI Biomarkers (TIMP-2 and IGFBP-7) are measured using the Nephrocheck® kit. Inflammatory markers include: CRP, D-dimer, Ferritin, IL-6, IL-8, GM-CSF, MCP 1 and TNF-α.

In addition to biological samples collected for the daily routine laboratory assessments performed at the Central laboratory, biological samples for research purposes are collected, including:
2 tubes 5 ml of serum
1 tube 3 ml of plasma
urine 30 ml

These samples are used to assess additional inflammatory cytokines and urinary biomolecules in order to obtain a more comprehensive characterization of patients enrolled, to better evaluate response to treatment, to provide more information in the follow-up and more importantly, to discover new potential biomarkers that could be useful for early diagnosis of sepsis-induced AKI. The analysis is performed by ELISA test and protein arrays.

On therapy visits (Treatment period): Treatment period is defined as from the start of treatment. The visit is planned at Day 3, Day 6 and Day 9. A final visit is planned on Day 30. The following procedures are performed during the therapy visits:
Recording of adverse events and concomitant medications
Review of appropriate laboratory information
Physical examination
Vital signs (pulse, blood pressure and oral, auricular, axillary, or core temperature) will be assessed
Record adverse events and concomitant medications continually
Complete blood count (CBC)—includes white blood cell count (WBC) with differential, platelet count, red blood cell count (RBC), haemoglobin (Hb), hematocrit (Hct).
Fasting chemistry panel/electrolytes: includes sodium, potassium, chloride, blood urea nitrogen (BUN; or urea), serum creatinine, calculated clearance creatinine (CKD-EPI),
glucose, calcium, phosphorus, total protein, uric acid, AST, ALT, γGT, ALP, total and direct bilirubine, albumin, total cholesterol, HDL, LDL, triglycerides, LDH, CPK
ABG (for assessing respiratory and/or metabolic disorders)
ApoA-I (for pharmacokinetic and pharmacodynamic assessment)
Coagulation tests—includes prothrombin time (PT) (expressed as international normalized ratio [INR]), and partial thromboplastin time (PTT).
Urinalysis—includes specific gravity, pH, assessment of protein/albumin, glucose, ketones, and haemoglobin/blood.
Microalbumunuria and Proteinuria g/24 h
Serum or urine pregnancy test (for women of childbearing potential) within 7 days before randomization.
Pharmacokinetic and pharmacodynamic assessment will include apoA-I and total cholesterol levels.
Endotoxin levels are measured using the EAA™ kit. AKI Biomarkers (TIMP-2 and IGFBP-7) are measured using the Nephrocheck® kit. Inflammatory markers include: CRP, D-dimer, Ferritin, IL-6, IL-8, GM-CSF, MCP 1 and TNF-α.

In addition to biological samples collected for the daily routine laboratory assessments performed at the Central laboratory, biological samples for research purposes are collected, including
2 tubes 5 ml of serum
1 tube 3 ml of plasma
urine 30 ml

Clinical scores include the SOFA score (Table 2) and the KDIGO criteria for AKI assessment and staging (Table 3). Individual components of each score are documented.

TABLE 2
The Sequential Organ Failure Assessment (SOFA) score

| SOFA Score: | 0 | 1 | 2 | 3 | 4 |
|---|---|---|---|---|---|
| Respiration | | | | | |
| PaO₂/FIO₂ (mmHg) | ≥400 | <400 | <300 | <220 and | <100 and |
| | | | | mechanically | mechanically |
| | | | | ventilated | ventilated |
| Coagulation | | | | | |
| Platelets × 10³/mm³ | ≥150 | <150 | <100 | <50 | <20 |
| Liver | | | | | |
| Bilirubin (mg/dL) | <1.2 | 1.2-1.9 | 2.0-5.9 | 6.0-11.9 | ≥12.0 |
| Cardiovascular^{a} | | | | | |
| Hypotension | MAP ≥ 70 | MAP < 70 | Dopamine ≤ 5 or | Dopamine > 5 or | Dopamine > 15 or |
| | | | dobutamine | norepinephrine ≤ 0.1 | norepinephrine > 0.1 |
| | | | (any) | | |
| CNS | | | | | |
| Glasgow Coma Score | 15 | 13-14 | 10-12 | 6-9 | <6 |
| Renal | | | | | |
| Creatinine (mg/dL) | <1.2 | 1.2-1.9 | 2.0-3.4 | 3.5-4.9 or <500 | >5.0 or <200 |
| or urine output | | | | | |
| (mL/day) | | | | | |

MAP = Mean arterial pressure;
CNS = central nervous system;
SaO₂ = peripheral arterial oxygen saturation
^{a}Vasoactive medications administered for at least 1 hr (dopamine and norepinephrine μg/kg/min)

TABLE 3
KDIGO classification for AKI

| Stage | Serum creatinine | Urine output |
|---|---|---|
| 1 | 1.5-1.9 times baseline | <0.5 ml/kg/h for 6-12 hours |
| | OR ≥0.3 mg/dl (≥26.5 μmol/l) | |
| | increase | |
| 2 | 2.0-2.9 times baseline | <0.5 ml/kg/h for ≥12 hours |
| 3 | 3.0 times baseline | <0.3 ml/kg/h for ≥24 hours |
| | OR | OR |
| | Increase in serum creatinine to ≥4.0 | Anuria for ≥12 hours |
| | mg/dl (≥353.6 μmol/l) | |
| | OR | |
| | Initiation of renal replacement | |
| | therapy OR, in patients <18 years, | |
| | decrease in eGFR to <35 ml/min | |
| | per 1.73 m³ | |

Table 4 provides a summary of the study protocol of this Example.

TABLE 4
Overview of study protocol

| | | | | | | | | | | Final |
|---|---|---|---|---|---|---|---|---|---|---|
| | | Treatment visits | | | | | | | | visit |
| | Base- | Day 1 | | Day 2 | | Day 3 | | Day | Day | Day |
| Procedure | line | am | pm | am | pm | am | pm | 6 | 9 | 30 |
| CER-001 | | X | X | X | X | X | X | X | | |
| Dosing | | | | | | | | | | |
| Endotoxin | X | X | | X | | X | | X | X | X |
| IL-6 | X | X | | X | | X | | X | X | X |
| Additional | X | | | | | | | | | X |
| Inflam- | | | | | | | | | | |
| matory | | | | | | | | | | |
| Markers^{a} | | | | | | | | | | |
| SOFA Score | X | X | | X | | X | | X | X | X |
| RIFLE Score | X | X | | X | | X | | X | X | X |
| apoA-I and | | X^{b} | | | | | | | | |
| Total | | | | | | | | | | |
| Cholesterol | | | | | | | | | | |
| Safety Labs^{c} | X | | | | | | | | | X |
| Optional | X | X | | X | | X | | X | X | X |
| samples | | | | | | | | | | |
| Concomitant | X | X | | X | | X | | X | X | X |
| Medication | | | | | | | | | | |
| Monitoring | | | | | | | | | | |
| Adverse | X | X | | X | | X | | X | X | X |
| Event | | | | | | | | | | |
| Monitoring | | | | | | | | | | |

^{a}Includes CRP, D-dimer, Ferritin, IL-8, VCAM-1, ICAM-1, GM-CSF, MCP 1 and TNF-α.
^{b}On dosing days, drawn prior to and 2 hours after the start of each infusion.
^{c}Tested at local hospital laboratory.

Safety Evaluations: Safety evaluations are attained utilizing information collected from the following assessments: physical examination (including weight), vital signs (blood pressure, pulse, temperature), CBC with differential, platelet count, blood chemistries, and fasting lipid profiles [including HDL-cholesterol, LDL-cholesterol and Lipoprotein (a)], urea, glucose, 24 hour urine protein determination, serum creatinine and calculated creatinine clearance (CKD-EPI) and adverse events monitoring. All women of childbearing potential have a qualitative serum pregnancy test during pre-study screening/baseline evaluation and subsequently, if clinically indicated. Patients are monitored throughout the study for the occurrence of adverse events, that are recorded. Adverse events volunteered by the subject or discovered, as a result of general questioning by the investigator or by physical examination, are recorded. The duration (start and end dates), severity, cause and relationship to study medication, patient outcome, action taken, and an assessment of whether the event was serious are recorded for each reported adverse event.

Adverse events: Definitions

The term “adverse event,” is synonymous with the term “adverse experience,” which is used by the FDA. An adverse event (AE) is any untoward, undesired, unplanned clinical event in the form of signs, symptoms, disease, or laboratory or physiological observations occurring in a human being participating in a clinical study regardless of causal relationship. This includes the following:
Any clinically significant worsening of a pre-existing condition.
Any reoccurrence of a pre-existing condition.
An AE occurring from overdose of an investigator test article whether accidental or intentional (i.e., a dose higher than that prescribed by a health care professional for clinical reasons).
An AE occurring from abuse of an investigator test article (i.e., use for no clinical reasons).
An AE that has been associated with the discontinuation of the use of an investigator test article.

A procedure is not an AE, but the reason for a procedure may be an AE.

A “preexisting condition” is a clinical condition (including a condition being treated) that is diagnosed before the subject signs the informed consent form and that is documented as part of the subject's medical history. The questions concerning whether the condition existed before the start of the active phase of the study and whether it has increased in severity and/or frequency are used to determine whether an event is a treatment-emergent adverse event (TEAE). An AE is considered to be treatment emergent if (1) it was not present when the active phase of the study began and is not a chronic condition that is part of the subject's medical history, or (2) it was present at the start of the active phase of the study or as part of the subject's medical history, but the severity or frequency increased during the active phase. The active phase of the study begins at the time of the first dose of the drug.

A “serious adverse event” is any AE occurring at any dose that meets 1 or more of the following criteria:
Results in death
Is life-threatening (see below)
Requires in subject hospitalization or prolongation of an existing hospitalization (see below)
Results in a persistent or significant disability or incapacity (see below)
Results in a new malignancy
Results in a congenital anomaly or birth defect

Additionally, important medical events that may not result in death, be life-threatening, or require hospitalization may be considered SAEs when, based on appropriate medical judgment, they may jeopardize the subject and may require medical or surgical intervention to prevent one of the outcomes listed above. Examples of such events include allergic bronchospasm requiring intensive treatment in an emergency room or at home, blood dyscrasias or convulsions that do not require hospitalization, or development of drug dependency or drug abuse.

A “life-threatening adverse event” is any AE that places the subject at immediate risk of death from the event as it occurred. A life-threatening event does not include an event that might have caused death had it occurred in a more severe form but that did not create an immediate risk of death as it actually occurred. For example, drug-induced hepatitis that resolved without evidence of hepatic failure would not be considered life-threatening, even though drug-induced hepatitis of a more severe nature can be fatal.

Hospitalization or prolongation of a hospitalization is a criterion for considering an AE to be serious. In the absence of an AE, the participating investigator should not report hospitalization or prolongation of hospitalization on a form. This is the case in the following situations: Hospitalization or prolongation of hospitalization is needed for a procedure required by the protocol. Day or night survey visits required by the protocol are not considered serious.

Timing for reporting serious adverse events: Any SAE, regardless of causal relationship, is reported to medical monitor immediately (no later than 24 hours after the investigator becomes aware of the SAE) by faxing a completed serious adverse event form. Follow-up information relating to an SAE is reported to medical monitor (or designee) within 24 hours of receipt by the investigator by faxing a completed serious adverse event form. The subject is observed and monitored carefully until the condition resolves or stabilizes or its cause is identified. Any emergency is reported to medical monitor (or designee) immediately (within 24 hours) by contacting a medical monitor.

Reportable events/information: An AE or SAE can occur from the time that the subject signs the informed consent form to 15 days from the subject's last dose, regardless of test article or protocol relationship. This includes events that emerge during the screening and placebo run-in periods. All AEs and SAEs are recorded on source documents and recorded on CRFs. All AEs and SAEs that occur after the screening period are recorded on the CRFs.

For SEAs: The investigator provides all documentation pertaining to the event (e.g., additional laboratory tests, consultation reports, discharge summaries, postmortem reports, etc.) to the Medical monitor in a timely manner. Reports relative to the subject's subsequent course are submitted to the investigator until the event has subsided or, in case of permanent impairment, until the condition stabilizes.

The following events are recorded and reported in the same time frame and following the same process as for SAEs:

Test article abuse and overdose (i.e. use for nonclinical reasons) with or without AEs. An overdose is a dose higher than that prescribed by a health care professional for clinical reasons. It is up to the participating investigator to decide whether a dose is an overdose.

Inadvertent or accidental exposure to test article with or without an AE.

Post study test article-related SAEs.

SAEs occurring after unauthorized or accidental use in persons not participating in the study.

Abnormal biological or vital signs values that are considered clinically relevant by the participating investigator. These are reported in the same time frame and following the same process as for an AE or an SAE

Recording and reporting: At each required study visit, all AEs that have occurred since the previous visit are recorded in the adverse event record of the subject's CRF. The information recorded is based on the signs or symptoms detected during the physical examination and clinical evaluation of the subject. In addition to the information obtained from those sources, the subject is asked the following non specific question: “How have you been feeling since your last visit?” Signs and symptoms are recorded using standard medical terminology. The health outcomes assessment surveys administered to study subjects are intended to explore the subject's own perceptions about their quality of life. However, the investigator reviews the survey for the presence of potential AEs or SAEs and considers the subject's perceptions when determining the occurrence of an AE or SAE. The subject's assessments are not intended to be influenced by the clinical investigator. Every effort is made to maintain an unbiased assessment. The following AE information is included (when applicable): the specific condition or event and direction of change; whether the condition was pre-existing (i.e. an acute condition present at the start of the study or history of a chronic condition) and, if so, whether it has worsened (in severity and/or frequency); the dates and times of occurrence; severity; causal relationship to test article; action taken; and outcome. Any laboratory abnormality, which in the opinion of the investigator is clinically significant, is reported as an AE.

The causal relation between an AE and the test article is determined by the investigator on the basis of his or her clinical judgement and the following definitions:
Definitely related: Event can be fully explained by administration of the test article.
Probably related: Event is most likely to be explained by administration of the test article, rather than the subject's clinical state or other agents/therapies.
Possibly related: Event may be explained by administration of the test article, or by the subject's clinical state or other agents/therapies.
Probably not related: Event is most likely to be explained by the subject's clinical state or other agents/therapies, rather than the test article.
Definitely not related: Event can be fully explained by the subject's clinical state or other agents/therapies.

When assessing the relationship between administration of a test article and an AE, the following are considered:
Temporal relationship between administration of the test article and the AE
Biological plausibility of relationship
Subject's underlying clinical state or concomitant agents and/or therapies

When applicable, whether the AE abates on discontinuation of the test article

When applicable, whether the AE reappears on repeat exposure to the test article SAEs that are not test article-related may nevertheless be considered by the participating investigator or the medical monitor (or designee) to be related to the conduct of the clinical study, i.e., to a subject's participation in the study. For example, a protocol-related SAE may be an event that occurs during a washout period or that is related to a procedure required by the protocol. The severity of AEs is assessed according to the National Cancer Institute (NCI) Common Toxicity Criteria for Adverse Events (CTCAE) version 5.0. The following definitions are used for toxicities that are not defined in the NCI CTCAE:
Mild (Grade 1): The AE is noticeable to the subject but does not interfere with routine activity. The AE does not require discontinuing administration or reducing the dose of the test article.
Moderate (Grade 2): The AE interferes with routine activity but responds to symptomatic therapy or rest. The AE may require reducing the dose but not discontinuing administration of the test article.
Severe (Grade 3): The AE significantly limits the subject's ability to perform routine activities despite symptomatic therapy. In addition, the AE leads to discontinuing administration or reducing the dose of the test article.
Life-Threatening (Grade 4): The AE requires discontinuing administration of the test article. The subject is at immediate risk of death.

### 6.2.2. Results

A first cohort of 10 subjects have been treated.

Cohort 1

| Treatment Group | N | Nephrology | ICU |
|---|---|---|---|
| Standard of Care | 2 | 2 | 0 |
| CER-001 5 mg/kg | 3 | 1 | 2 |

Cohort 1

| Treatment Group | N | Nephrology | ICU |
|---|---|---|---|
| CER-001 10 mg/kg | 3 | 2 | 1 |
| CER-001 20 mg/kg | 2 | 2 | 0 |

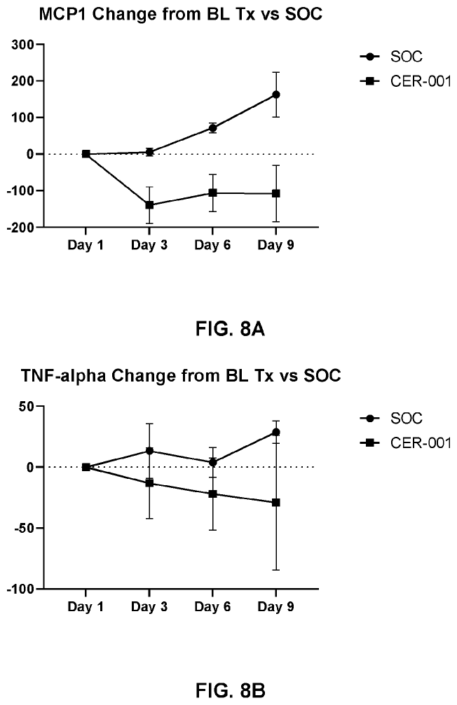

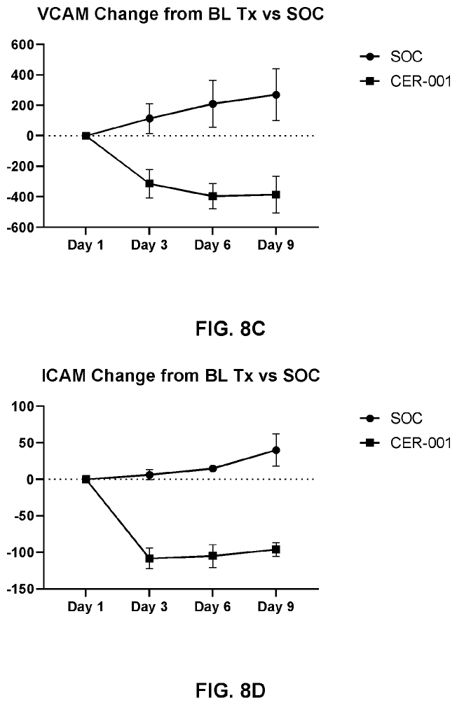

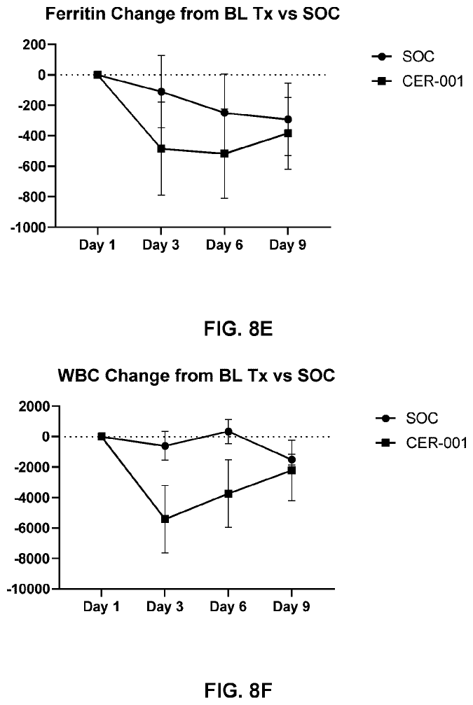

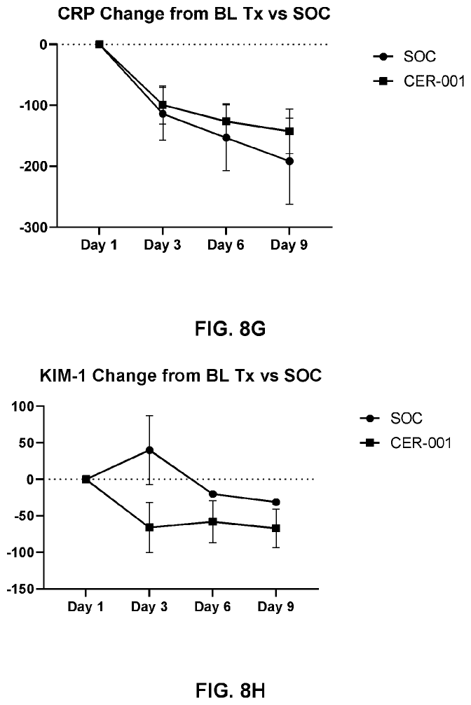

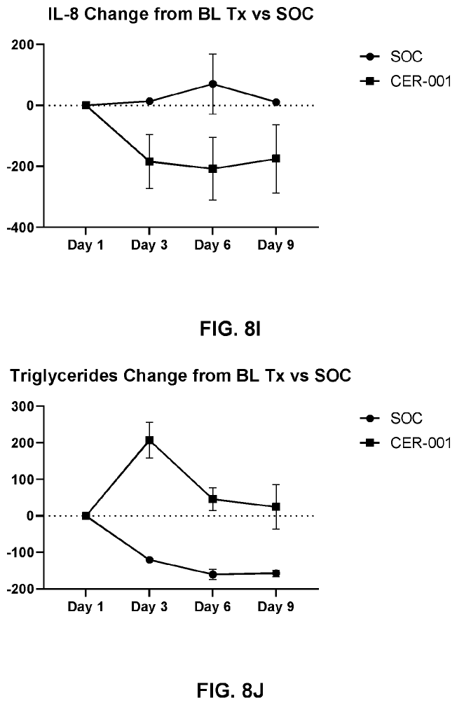

Results from the first cohort are shown in FIGS. 8A-8J.

Compared to standard of care therapy, CER-001 rapidly improved biomarkers of inflammation, leukocytosis, and endothelial dysfunction, preventing subjects' decline into acute kidney injury. CER-001 treatment was well tolerated at all dose levels (5, 10 and 20 mg/kg, twice a day). No treatment-related serious side effects were seen in this critically ill patient population.

### 6.2.2.1. VCAM and ICAM

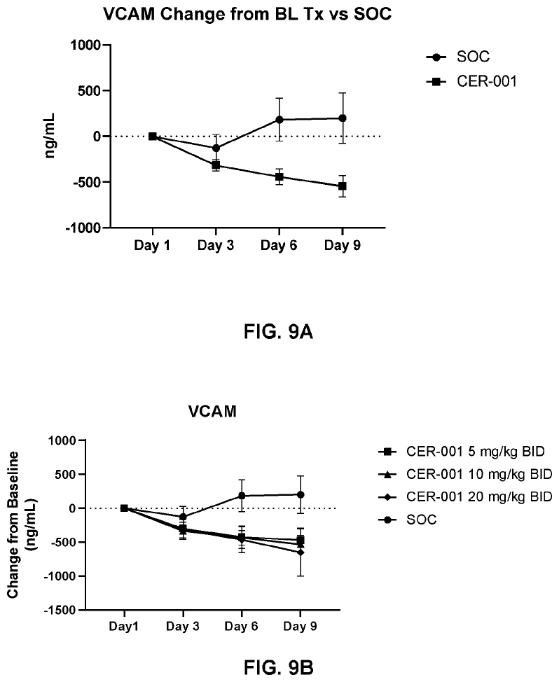

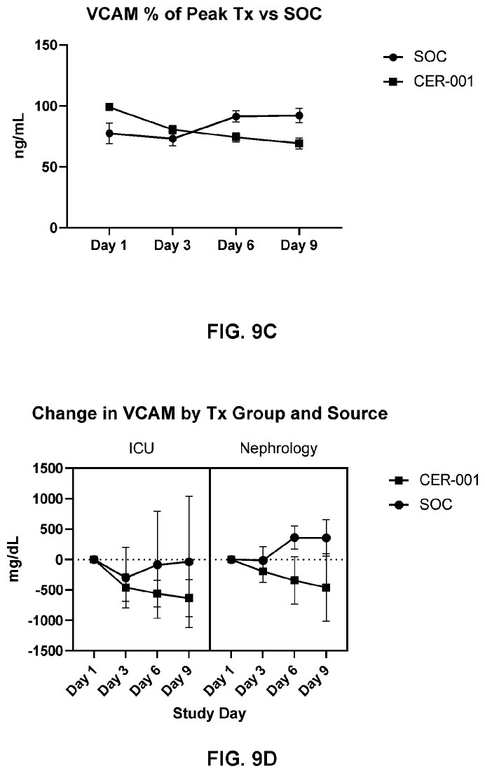

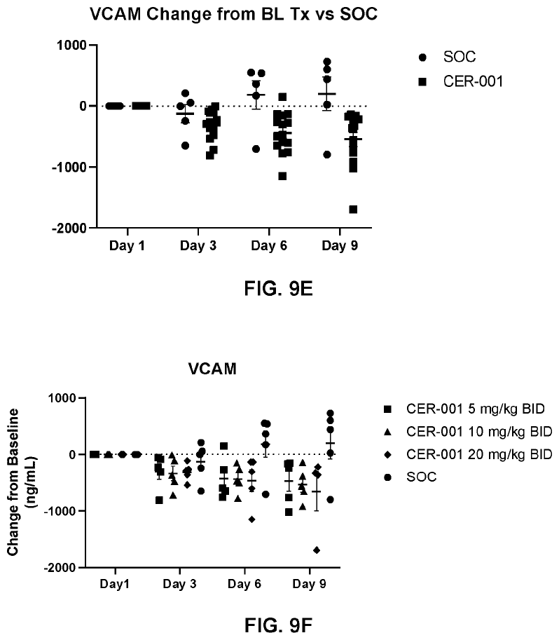

The study was continued with a total of 20 subjects, n=5 per treatment group. FIGS. 9A-FIG. 9F show VCAM changes for the standard of care group and the CER-001 groups measured by ELISA. Statistically significant differences were assessed by using a mixed model ANOVA (ns: p>0.05). FIG. 9A: VCAM changes from baseline for SOC group and aggregated CER-001 groups. FIG. 9B: VCAM changes from baseline for SOC group and each study group. FIG. 9C: VCAM changes, reported as a percentage relative to peak VCAM levels (peak=100%), for SOC group and aggregated CER-001 groups. The treatment x study day effect relative to peak was p<0.0001. FIG. 9D: VCAM changes for SOC group and aggregated CER-001 groups, broken out by whether the subject was enrolled from the ICU or the nephrology department of the center. FIG. 9E: VCAM changes from baseline for each subject in SOC group and aggregated CER-001 groups. FIG. 9F: VCAM changes from baseline for each subject in each group.

Generally, treatment regimens providing CER-001 in addition to the SOC reduced VCAM more than the SOC alone. Results are summarized in the following table:

| Treatment Effect | Overall | Day 3 | Day 6 | Day 9 |
|---|---|---|---|---|
| CER-001 vs SOC | p = 0.0090 | NS | p = 0.0520 | p = 0.0510 |
| Treatment Groups | p = 0.0870 | | | |
| SOC vs 5 | | NS | p = 0.0687 | p = 0.0805 |
| SOC vs 10 | | NS | p = 0.0556 | p = 0.0558 |
| SOC vs 20 | | NS | p = 0.0646 | NS |
| 5 vs 10 | | NS | NS | NS |
| 5 vs 20 | | NS | NS | NS |
| 10 vs 20 | | NS | NS | NS |

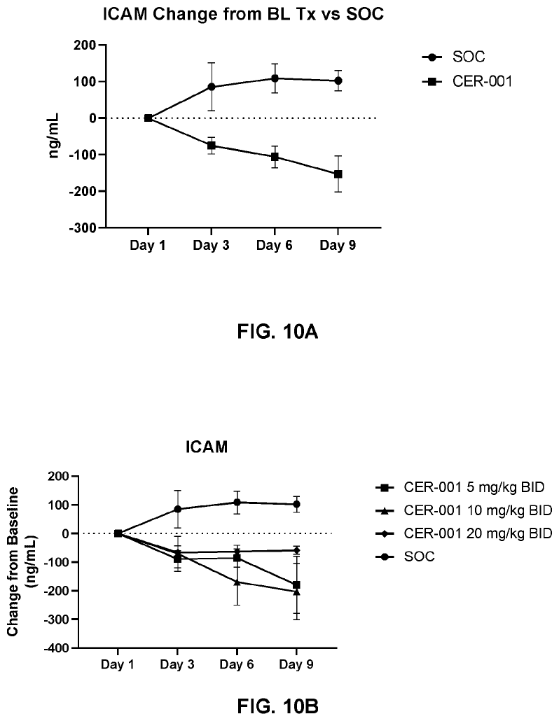

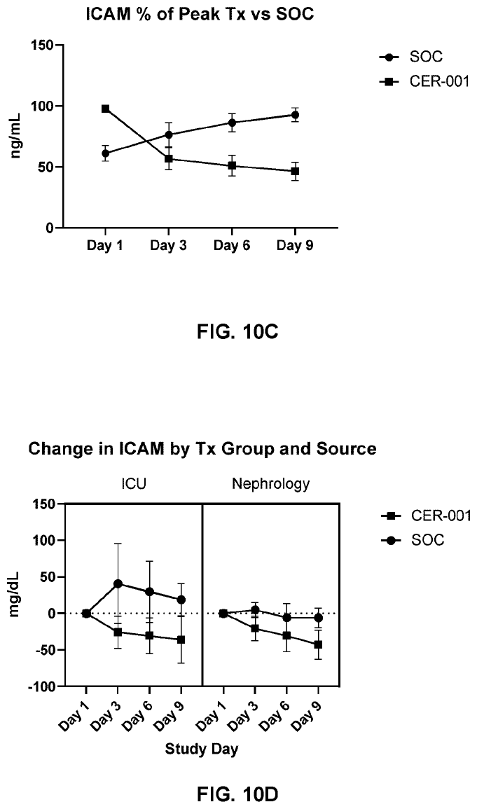

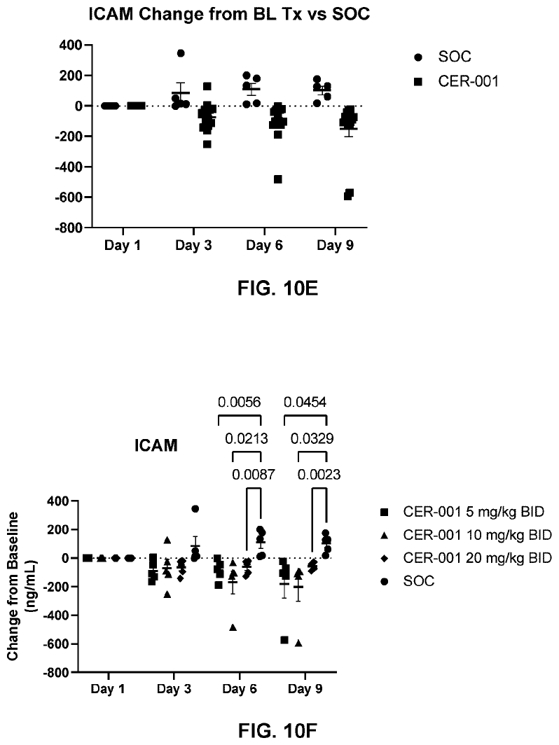

FIG. 10A-FIG. 10F show ICAM changes for the standard of care group (SOC group) and the experimental groups (CER-001 groups) measured by ELISA. Statistically significant differences were assessed by using a mixed model ANOVA (ns: p>0.05). FIG. 10A: ICAM changes from baseline for SOC group and aggregated CER-001 groups. FIG. 10B: CAM changes from baseline for SOC group and each study group. FIG. 10C: ICAM changes, reported as a percentage relative to peak ICAM levels (peak=100%), for SOC group and aggregated CER-001 groups. The treatment x study day effect relative to peak was p<0.0001. FIG. 10D: ICAM changes for SOC group and aggregated CER-001 groups, broken out by whether the subject was enrolled from the ICU or the nephrology department of the center. FIG. 10E: ICAM changes from baseline for each subject in SOC group and aggregated CER-001 groups. FIG. 10F: ICAM changes from baseline for each subject in each study group.

Generally, treatment regimens providing CER-001 in addition to the SOC reduced ICAM more than the SOC alone. Results are summarized in the following table:

| Treatment | | | | |
|---|---|---|---|---|
| Effect | Overall | Day 3 | Day 6 | Day 9 |
| CER-001 vs | p = 0.0015 | NS | p = 0.0020 | p = 0.0003 |
| SOC | | | | |
| Treatment | p = 0.0127 | | | |
| Groups | | | | |
| SOC vs 5 | | p = 0.0552 | p = 0.0056 | p = 0.0454 |
| SOC vs 10 | | NS | p = 0.0213 | p = 0.0329 |
| SOC vs 20 | | p = 0.0831 | p = 0.0087 | p = 0.0023 |
| 5 vs 10 | | NS | NS | NS |
| 5 vs 20 | | NS | NS | NS |
| 10 vs 20 | | NS | NS | NS |

### 6.2.2.2. White Blood Cells

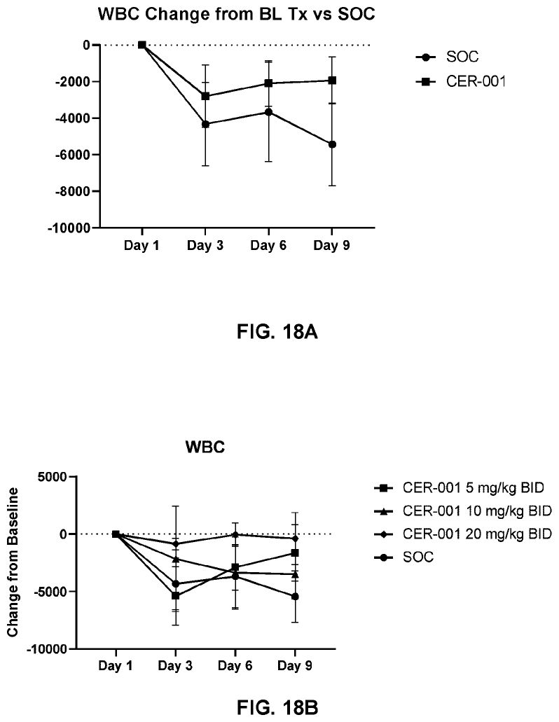

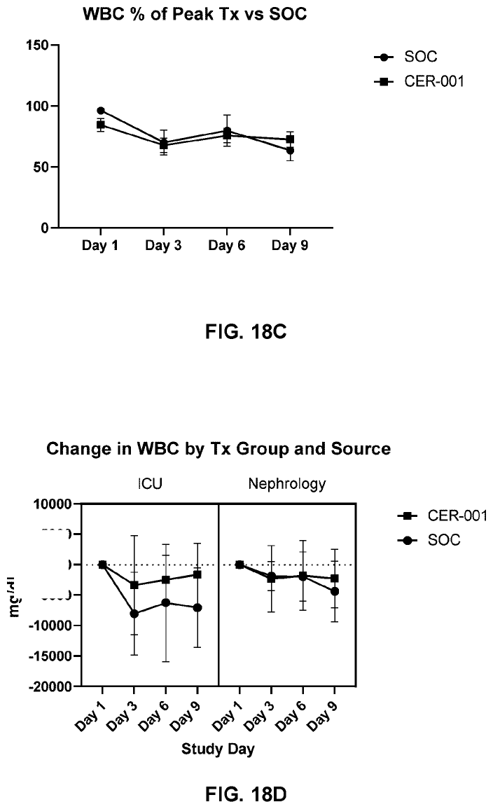

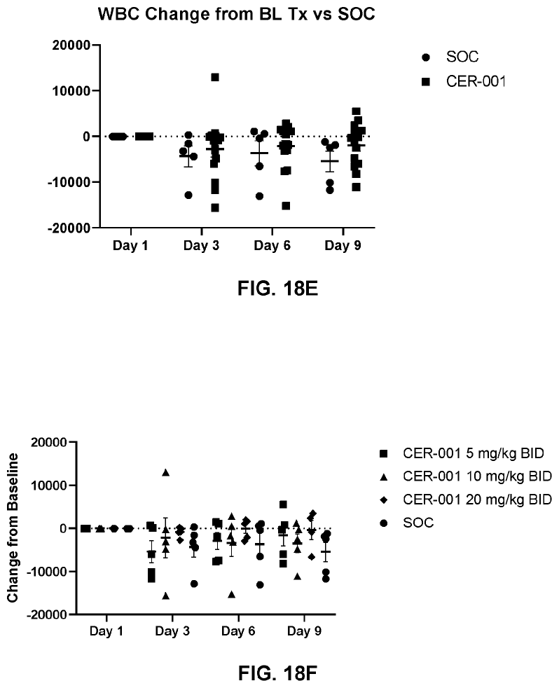

FIG. 18A-FIG. 18F show white blood cell count changes for the standard of care (SOC) group and the CER-001 groups. FIG. 18A: white blood cell count changes from baseline for SOC group and aggregated CER-001 groups. FIG. 18E shows the individual data points summarized in FIG. 18A. FIG. 18B: white blood cell count changes from baseline for SOC group and each CER-001 group. FIG. 18F shows the individual data points summarized in FIG. 18B. FIG. 18C: white blood cell count changes, reported as a percentage relative to peak white blood cell counts (peak=100%), for SOC group and aggregated CER-001 groups. The treatment x study day effect relative to peak was p=0.5492. FIG. 18D: white blood cell count changes for SOC group and aggregated CER-001 groups, broken out by whether the subject was enrolled from the ICU or the nephrology department of the center. Units for FIG. 18A, FIG. 18B, and FIG. 18D: cells per microliter.

### 6.3. Example 3: CER-001 Therapy for Treating CRS Secondary to Covid-19 Infection

COVID-19 is infects host cells through binding of the viral spike protein (SARS-2-S) to the cell-surface receptor angiotensin-converting enzyme 2 (ACE2), and the HDL scavenger receptor B type 1 (SR-B1) facilitates ACE2-dependent entry of the virus. (Wei et al., Nature Metabolism doi.org/10.1038/s42255-020-00324-0). Without being bound by theory, it is believed that lipid binding protein-based complexes such as CER-001 may provide a therapeutic benefit (e.g., reducing the severity and/or duration of CRS) in subjects having a COVID-19 infection through competitive binding to SR-B1, thereby limiting the virus's ability to infect additional cells.

A pilot study is conducted to investigate the safety and efficacy of seven CER-001 infusions in patients with CRS secondary to COVID-19 infection. The study consists of 9 visits:
Pre-Dosing (Baseline) Visit: Assessment of baseline inflammatory markers and safety labs.
Dosing Visits: Seven doses (Doses 1 through 7) are administered as a once daily infusion over a 7-day period. IL-6 is measured daily from a pre-infusion sample.
Follow-Up Visit: Patients have their final evaluation on Day 8. Inflammatory markers and safety labs are measured.

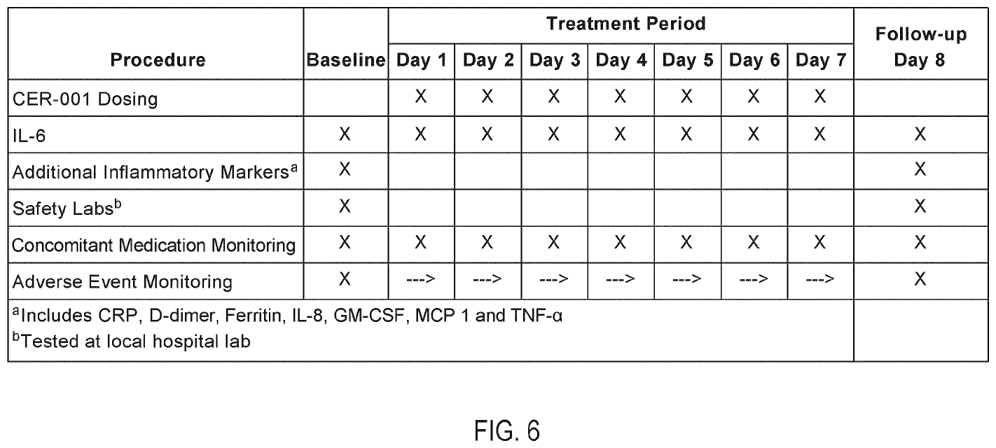

A flowchart for the study is shown in FIG. 6.

### 6.3.1. Selection of Study Subjects

### 6.3.1.1. Inclusion Criteria

Eligible patients meeting the following criteria are enrolled into the study:
1. Male or non-pregnant female adult >18 years of age at time of enrollment.
2. Has laboratory-confirmed novel coronavirus (COVID-19) infection as determined by polymerase chain reaction (PCR), or other commercial or public health assay in oropharyngeal or anal specimen within 72 hours prior to hospitalization.
3. Illness of any duration, and at least one of the following:
   a. Radiographic infiltrates by imaging (chest x-ray, CT scan, etc.), OR
   b. Clinical assessment (evidence of rales/crackles on physical examination) AND SpO2≤93% on room air, OR
   c. Requiring mechanical ventilation and/or supplemental oxygen, OR
   d. Sustained fever in the past 24 hours and unresponsive to NSAID or steroid
4. Serum IL-6≥3 times the upper limit of normal
5. Females of childbearing potential that agree and commit to use an acceptable form of birth control for the entire study. Acceptable forms of birth control for this study are defined as a barrier method plus hormonal therapy (implants, injections, oral contraceptives and IUDs) or abstinence.

### 6.3.1.1. Exclusion Criteria

Patients meeting the following criteria are excluded from the study:
1. Patients weighing more than 100 kg
2. Alanine transaminase/aspartate transaminase (ALT/AST) >5 times the upper limit of normal.
3. Stage 4 severe chronic kidney disease or requiring dialysis (i.e. estimated glomerular filtration rate (eGFR)<30 ml/min/1.73 m\hat{ }2)
4. Hemoglobin<80 g/L
5. Leukocytes<2.0×10\hat{ }9
6. Platelets<50×10\hat{ }9
7. Pregnancy or breast feeding.
8. Anticipated transfer to another hospital which is not a study site within 72 hours.
9. Expected life span does not exceed 7 days.
10. Patients who have used an investigational agent within 30 days of the first dose of CER-001.

### 6.3.1.2. Restrictions During the Study

There are no patient restrictions other than those outlined in the Inclusion/Exclusion criteria above.

### 6.3.1.3. Withdrawal Criteria

Reasons for withdrawal of a patient from study drug may include, but are not limited to, the following:
Investigator's request, for safety reasons, such as severe adverse reactions;
Investigator's request, for other reasons, such as patient non-compliance;
Patient's request, for tolerability reasons;
Patient's request, for other reasons, such as withdrawal of informed consent;

Discontinuation of study drug alone does not constitute discontinuation or withdrawal from the study. Patients continue to be followed as though they had completed the treatment phase. Patients who prematurely discontinue study medication (e.g., prior to completion of the 7th dose) undergo end of study evaluations whenever possible.

### 6.3.2. Treatment of Patients

### 6.3.2.1. Investigational Product

CER-001 is provided frozen in 20 mL vials containing approximately 18 mL of product at a concentration of 8 mg/mL (ApoA-I content). CER-001 is dosed by weight. All doses are thawed and then diluted with normal saline to a volume of 250 mL.

Dosing occurs at each of the seven dosing visits. At each of these visits, patients are given a single IV infusion CER 001 (20 mg/kg) over a period of 24 hours using an infusion pump. Patients are pretreated with antihistamine prior to each CER-001 dose (e.g. dexchlorpheniramine 5 mg or hydroxyzine 100 mg) to avoid any potential infusion reactions.

### 6.3.2.2. Interruption or Discontinuation of Study Medication

Patients are interrupted or discontinued from study medication if any of the following occur:
Any drug-related adverse event or other reason which, in the Investigator's opinion, jeopardize the patient's participation in the trial or the interpretation of trial data (e.g., severe inter-current illness requiring additional care measures or preventing further dosing)
Significant tolerability issues

At the time of study medication interruption, the study site documents the reason for drug interruption. The patient continues to be followed clinically and all attempts are made to re-institute study medication within 2 days of the study drug interruption if not otherwise contraindicated.

### 6.3.3. Concomitant Treatments

All non-experimental treatments are allowed to be administered concomitantly during the patient's participation in this study. Any medication the patient takes, other than study drugs specified per protocol, is considered a concomitant medication and is recorded in the study records.

### 6.3.4. Prohibited Medication

There are no excluded medications.

### 6.3.5. Monitoring Patient Compliance

CER-001 is administered in the hospital under direct observation.

### 6.3.6. Assessment of Efficacy

### 6.3.6.1. Efficacy Assessments

Inflammatory markers include: CRP, D-dimer, Ferritin, IL-6, IL-8, GM-CSF, MCP 1 and TNF-α.

### 6.3.6.2. Efficacy Parameters

### (a) Primary Efficacy Parameters

The primary efficacy parameter is the change in IL-6 from baseline to Day 8. Baseline is defined as the average of the measurements taken at the baseline visit and prior to dosing on Day 1.

### (b) Secondary Efficacy Parameters

Secondary efficacy parameters include changes to the inflammatory markers CRP, D-dimer, Ferritin, IL-8, GM-CSF, MCP 1 and TNF-α from baseline to Day 8.

### 6.3.7. Assessment of Safety

### 6.3.7.1. Safety Parameters

### (a) Pregnancy Tests (if Applicable)

Females of child bearing potential have a documented negative pregnancy test performed any time during hospitalization and prior to dosing.

### (b) Safety Laboratory Tests

Blood samples are drawn for chemistry and hematology analyses at two time points: baseline and Day 8. The following tests are performed by the local hospital laboratory:

| Chemistry Profile | Hematology |
|---|---|
| Albumin | White Blood Count |
| Alkaline Phosphatase | Red Blood Count |
| Alanine Aminotransferase (ALT/SGPT) | Hemoglobin |
| Aspartame Aminotransferase (AST/SGOT) | Hematocrit |
| Urea | Neutrophils |
| Calcium | Lymphocytes |
| Chloride | Monocytes |
| Bicarbonate | Eosinophils |
| Creatinine | Basophils |
| Glucose | Platelets |
| Potassium | |
| Sodium | |
| Total Bilirubin | |
| Total Protein | |

### 6.3.8. Results

IL-6 levels are reduced from baseline to day 8. Secondary efficacy parameters are also reduced from baseline to day 8, indicating that CER-001 therapy can be used to treat CRS and reduce serum levels of inflammatory markers.

### 6.4. Example 4: CER-001 Therapy for Treating CRS Secondary to Covid-19 Infection—Additional Treatment Protocol

This Example is a study of CER-001 therapy in COVID-19 patients with severe cytokine release syndrome and renal injury.

### 6.4.1. Selection of Subjects

### 6.4.1.1. Inclusion Criteria

Eligible patients meet the following criteria before they are enrolled into the study:
1. Male or non-pregnant female adult 18 years of age at time of enrollment.
2. Has laboratory-confirmed novel coronavirus infection as determined by polymerase chain reaction (PCR), or other commercial or public health assay in oropharyngeal or anal specimen within 72 hours prior to hospitalization.
3. Illness of any duration, and at least one of the following:
Radiographic infiltrates by imaging (chest x-ray, CT scan, etc.), OR
Clinical assessment (evidence of rales/crackles on physical examination) AND SpO2<93% on room air, OR
Requiring mechanical ventilation and/or supplemental oxygen, OR
Sustained fever in the past 24 hours and unresponsive to NSAID or steroid
4. Serum IL-6>3 times the upper limit of normal
5. Females of childbearing potential that agree and commit to use an acceptable form of birth control for the entire study. Acceptable forms of birth control for this study are defined as a barrier method plus hormonal therapy (implants, injections, oral contraceptives and IUDs) or abstinence.

### 6.4.1.2. Exclusion Criteria

Patients who meet any of the following criteria are excluded from this study.
1. Clinical history suggesting allergies to CER-001
2. Pregnancy or breast feeding.
3. Anticipated transfer to another hospital within 72 hours.
4. Expected life span does not exceed 7 days.
5. Patients who have used an investigational agent within 30 days of the first dose of CER-001.

### 6.4.2. Treatments

### 6.4.2.1. Treatments Administered

Patients are pretreated with antihistamine prior to each CER-001 dose (e.g. dexchlorpheniramine 5 mg or hydroxyzine 100 mg) to avoid any potential infusion reactions.

Patients receive IV infusion of CER-001 at the dosage of 15 mg/kg BID for 3 consecutive days. At the discretion of the investigator, patients may receive up to two additional doses.

Patients may be interrupted or discontinued from study medication if any of the following occur:
1. Any drug-related adverse event or other reason which, in the Investigator's opinion, jeopardizes the patient's participation in the trial or the interpretation of trial data (e.g., severe inter-current illness requiring additional care measures or preventing further dosing);
2. Significant tolerability issues.

At the time of study medication interruption, the study site documents the reason for drug interruption. The patient continues to be followed clinically and all attempts are made to re-institute study medication within 2 days of the study drug interruption if not otherwise contraindicated.

Reasons for withdrawal from study drug may include, but are not limited to, the following:
1. Investigator's request, for safety reasons, such as severe adverse reactions
2. Investigator's request, for other reasons, such as patient non-compliance
3. Patient's request, for tolerability reasons
4. Patient's request, for other reasons, such as withdrawal of informed consent

Discontinuation of study drug alone does not constitute discontinuation or withdrawal from the study. Patients continue to be followed as though they had completed the treatment phase. Patients who prematurely discontinue study medication (e.g., prior to completion of the 3th dose) undergo end of study evaluations whenever possible.

### 6.4.2.2. Dose Changes

In case of clinical needs defined by the main investigator, the dose of the drug may be reduced or increased

### 6.4.2.3. Concomitant Medications/Therapies

All non-experimental treatments are allowed to be administered concomitantly during a patient's participation in this study. Any medication the patient takes, other than study drugs specified per protocol, is considered a concomitant medication and is recorded in the study records.

### 6.4.3. Study Assessments

The following procedures are performed during the Baseline visit. The following tests are performed by the local hospital laboratory.
1. Informed consent
2. Medical history includes recording past and present illnesses and collection of the subject's demographic data (birth date, sex, and race).
3. Physical examination with a review of systems, height and weight, BMI and wait circumference
4. Vital signs (pulse, blood pressure, and oral, auricular, axillary, or core temperature).
5. Review of inclusion/exclusion criteria.
6. Adverse events are recorded starting from the time informed consent is obtained.
7. Prior medications are collected from 4 weeks before the first dose of test article. All current medications are recorded.
8. Complete blood count (CBC)—includes white blood cell count (WBC) with differential, platelet count, red blood cell count (RBC), haemoglobin (Hb), hematocrit (Hct).
9. Fasting chemistry panel/electrolytes: includes sodium, potassium, chloride, blood urea nitrogen (BUN; or urea), serum creatinine, calculated clearance creatinine (CKD-EPI), glucose, calcium, phosphorus, total protein, uric acid, AST, ALT, QGT, ALP, total and direct bilirubine, albumin, total cholesterol, HDL, LDL, triglycerides, LDH, CPK,
10. ABG (for assessing respiratory and/or metabolic disorders)
11. ApoA-I (for pharmacokinetic and pharmacodynamic assessment)
12. Coagulation tests—includes prothrombin time (PT) (expressed as international normalized ratio [INR]), and partial thromboplastin time (PTT).
13. Urinalysis—includes specific gravity, pH, assessment of protein/albumin, glucose, ketones, and haemoglobin/blood.
14. Microalbumunuria and Proteinuria g/24 h
15. Serum or urine pregnancy test (for women of childbearing potential) within 7 days before randomization.
16. Pharmacokinetic and pharmacodynamic assessment include apoA-I and total cholesterol levels.
17. Inflammatory markers include CRP, PCT, D-dimer, Ferritin, IL-6, IL-8, GM-CSF, MCP 1 and TNF-α.

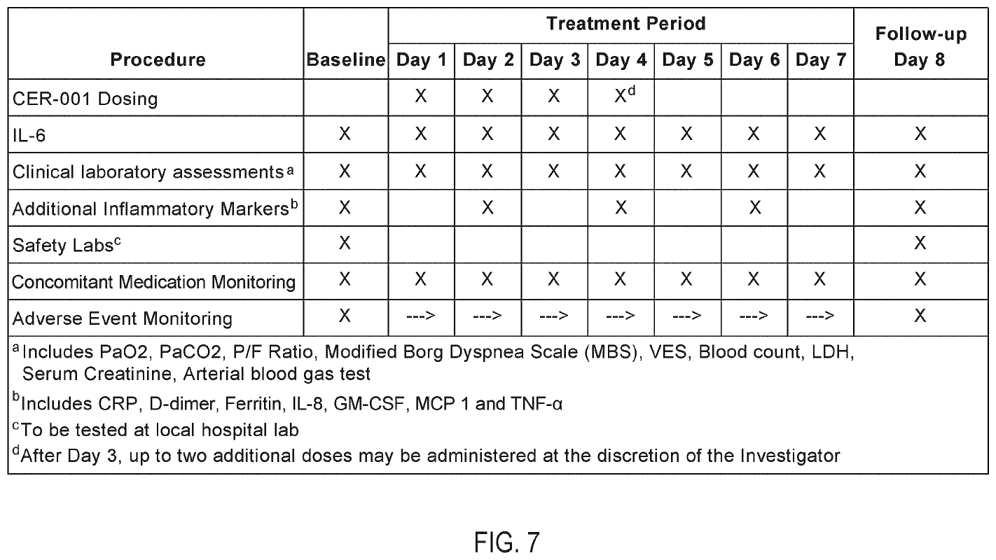

Clinical and laboratory parameters are monitored from baseline to the Final visit at Day 8 as reported in FIG. 7 and include the following procedures:
1. Recording of adverse events and concomitant medications
2. Review of appropriate laboratory information
3. Physical examination
4. Vital signs (pulse, blood pressure and oral, auricular, axillary, or core temperature) are assessed
5. Record adverse events and concomitant medications continually
6. Complete blood count (CBC)—includes white blood cell count (WBC) with differential, platelet count, red blood cell count (RBC), haemoglobin (Hb), hematocrit (Hct).
7. Fasting chemistry panel/electrolytes: includes sodium, potassium, chloride, blood urea nitrogen (BUN; or urea), serum creatinine, calculated clearance creatinine (CKD-EPI), glucose, calcium, phosphorus, total protein, uric acid, AST, ALT, QGT, ALP, total and direct bilirubine, albumin, total cholesterol, HDL, LDL, triglycerides, LDH, CPK
8. ABG (for assessing respiratory and/or metabolic disorders)
9. ApoA-I (for pharmacokinetic and pharmacodynamic assessment)
10. Coagulation tests—includes prothrombin time (PT) (expressed as international normalized ratio [INR]), and partial thromboplastin time (PTT).
11. Urinalysis—includes specific gravity, pH, assessment of protein/albumin, glucose, ketones, and haemoglobin/blood.
12. Microalbumunuria and Proteinuria g/24 h
13. Inflammatory markers include CRP, PCT, D-dimer, Ferritin, IL-6, IL-8, GM-CSF, MCP 1 and TNF-α

### 6.4.4. Adverse Event (AE) Reporting

An AE is any untoward medical occurrence associated with the use of the investigational product (active or placebo drug, biologic, or device) in a clinical investigation patient, which does not necessarily have a causal relationship with the product. An AE can, therefore, be any unfavorable and unintended sign (e.g., an abnormal laboratory finding), symptom, or disease temporally associated with the use of an investigational product, whether or not considered related to the investigational product.

Adverse events may include:
Symptoms described by the patient
Clinically significant changes in the patient's physical exam or other signs observed by the Investigator or medical staff
Test abnormalities (laboratory tests) that reflect a change from baseline and/or that may result in changes in administration of investigational product or in an alteration in medical care (diagnostic or therapeutic)
Conditions present at baseline that have either worsened or recurred following resolution

The patients are evaluated for new AEs and the status of existing AEs at each study visit.

### 6.4.5. Results

IL-6 levels and other inflammatory markers are reduced from baseline to day 8.

### 6.5. Example 5: CER-001 Therapy for Treating Ischemia/reperfusion AKI

This Example is a study of CER-001 therapy for treating ischemia/reperfusion AKI.

### 6.5.1. Materials and Methods

Pigs, with a body weight of 45-60 kg, are fasted for 24 hours before the study. All animals are premedicated with an intramuscular mixture of azaperone (8 mg kg⁻¹) and atropine (0.03 mg kg⁻¹) to reduce pharyngeal and tracheal secretions and prevent post-intubation bradycardia. After anesthesia, both kidneys are approached through a midline abdominal incision. Then, the renal arteries and vein are isolated and a vessel loop is positioned around the renal artery with a right angle clamp. Warm ischemia is induced for 60 minutes by pulling on the vessel loop. Ischemia is followed by 3 hours of reperfusion, with one half of the animals receiving CER-001 administered directly through the renal artery 5 minutes before the beginning of reperfusion. The animals are euthanized after 24 hours by an IV administration of 1 mL/kg BW pentobarbital. Kidneys are then harvested for analysis.

### 6.5.2. Results

CER-001 attenuates ischemia/reperfusion AKI.

### 6.6. Example 6: Lipid Binding Protein Molecule Therapy in a Model of LPS-Induced Vascular Endothelial Injury

The ability of the ApoA-I containing complex CER-001 to mitigate sepsis-induced injury to vascular endothelium was evaluated in a lipopolysaccharide (LPS)-induced in vitro model.

### 6.6.1. Materials and Methods

### 6.6.1.1. Cell Culture

Human umbilical vein endothelial cells (HUVEC, EC) were purchased from American Type Culture Collection (ATCC-LGC Standards S.r.l., Sesto San Giovanni, Milan, Italy). EC were maintained in their recommended medium, EndGro (Merck Millipore, Darmstadt, Germany).

Peripheral blood mononuclear cells (PBMCs) were isolated by gradient centrifugation with the Ficoll-Hypaque method from buffy coats of healthy donors (selected from a research repository) as previously described (Sallustio, et al., 2021, Nephrol Dial Transplant 36, 452-464). PBMCs were maintained in their recommended media (Ibid.).

When cells became confluent, they were stimulated with LPS 0.3 μg/ml, 4 μg/ml (E. Coli O111::B4, Sigma-Aldrich, Milan, Italy) and CER-001 50, 100, and 500 μg/ml for the indicated time period.

PBMCs culture supernatants were collected and analyzed by ELISA for TNF-α (R&D Systems, Minneapolis MN, USA)

### 6.6.1.2. Cell Proliferation Assay

EC and PBMCs were incubated with LPS at 0.3 μg/ml and/or CER-001 at 50 and 500 μg/ml for 60 min and 24 hours. Proliferation rate was measured by MTT Cell Proliferation Assay Kit, according to the manufacturer instructions (Sigma Aldrich). Briefly, 3×10⁴ cells/well were seeded in a 96-well plate, and then cells were treated with LPS and CER-001 as indicated. Absorbance at 570 nm was then measured by a spectrophotometer.

### 6.6.1.3. Immunophenotypic Analysis

After stimulations, EC were permeabilized with IntraPrep kit (Instrumentation Laboratory) and incubated with unconjugated primary antibody p-ENOS (Abcam) for 25 minutes at 4° C. Cells were then washed and labeled with secondary Antibody AlexaFluor 488 (Molecular Probes) for 25 minutes at 4° C. Finally, cells were washed twice and resuspended in FACS buffer for acquisition.

PBMCs were stained with the following monoclonal antibody, CD14 Monoclonal Antibody (61 D3)-PE, (eBioscience™, Thermo Fisher Scientific, Italy), for 20 minutes in the dark at room temperature, washed twice, and resuspended in FACS buffer. Stained PBMCs were then acquired.

Data were obtained by using a FC500 (Beckman Coulter) flow cytometer and analyzed with Kaluza software. Three independent studies were performed for both EC and PBMCs. The area of positivity was determined by using an isotype-matched mAb, and in total, 104 events for each sample were acquired.

### 6.6.1.4. Statistical Analysis

Data shown are representative of three independent studies. Data are shown as mean±standard deviation (SD) and compared with the Student-t test.

### 6.6.2. Results

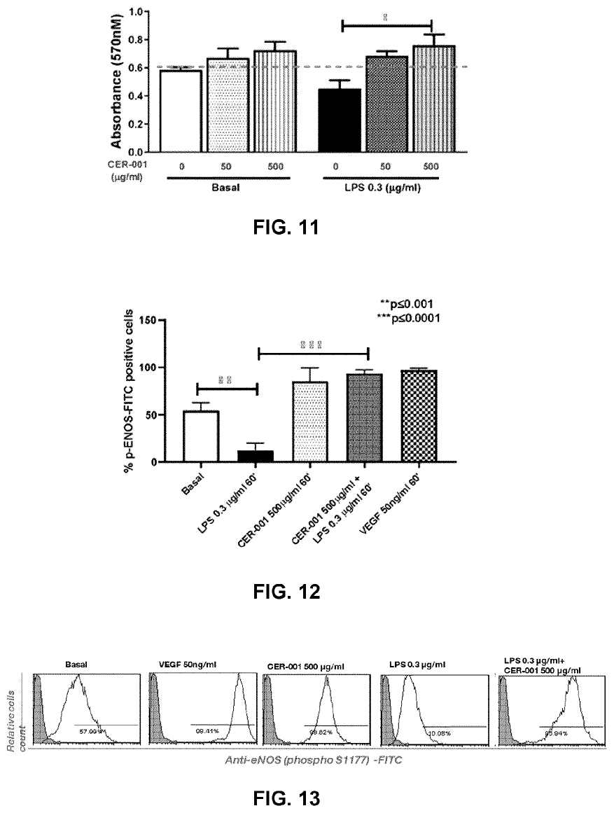

Effects of LPS and CER-001 on endothelial cells and the endothelial nitric oxide synthase (eNOS) activation were analyzed. The MTT cell viability assay results in FIG. 11 showed a slight reduction in proliferation after LPS stimulation. CER-001 at 50 and 500 μg/ml did not affect endothelial viability. Endothelial cells treated with LPS and CER-001, both at 50 and 500 μg/ml, increased the proliferation rate, in particular at the highest concentration, compared to LPS-stimulated cells.

Production of eNOS has been described as a marker of the vascular endothelial integrity (Zhao, et al., 2015, J Pharmacol Sci 129, 83-94). In the in vitro model of this Example, eNOS phosphorylation and activation (FIG. 12-FIG. 13) was altered by LPS and upregulated by CER-001. Specifically, a strong decrease of eNOS (phospho S1177) (p-ENOS) was observed after 60 min of LPS stimulation compared to basal and VEGF (positive control). CER-001 supplementation at 500 μg/ml completely reversed LPS effects. (In FIG. 13, representative data from one out of a total of three studies are shown. Histograms indicate p-ENOS expression levels).

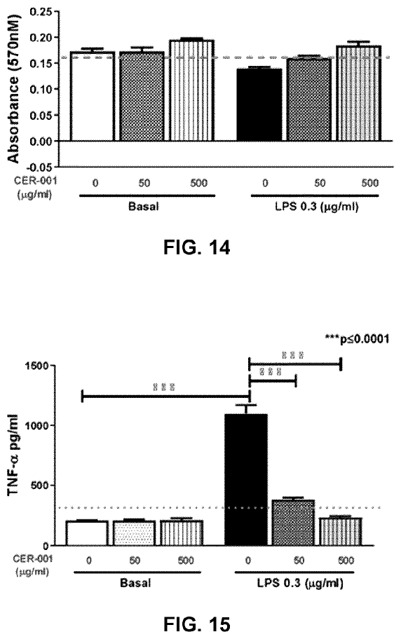

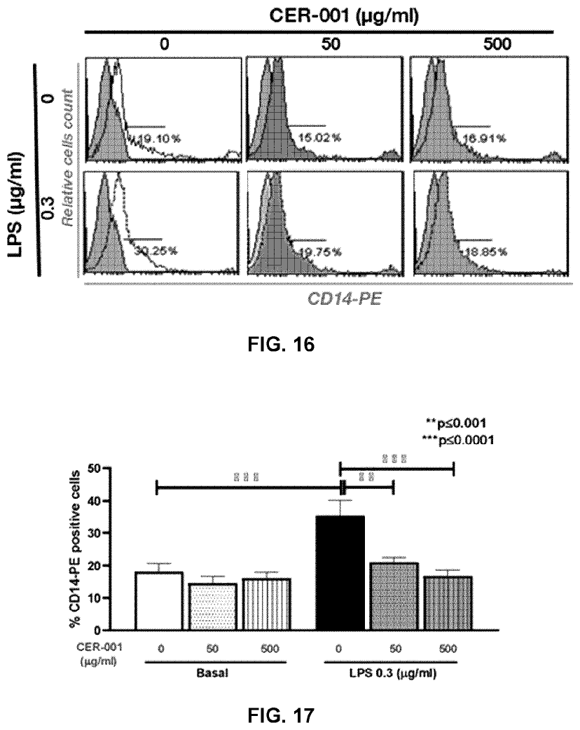

In addition, CER-001 modulated the response of peripheral blood mononuclear cells (PBMC) stimulated with LPS at 0.3 μg/ml and/or CER-001 at 50 and 500 μg/ml for 24 hours, decreasing mCD14 expression and TNF-α secretion. As shown in FIG. 14, MTT assay showed no significant difference in cell viability with respect to the basal for the above conditions. PBMC culture supernatants were analyzed by ELISA with results shown in FIG. 15. After 24 h from LPS stimulation, PBMCs increased TNF-α synthesis. Stimulation of PBMCs with CER-001 at 50 and 500 μg/m alone did not influence TNF-α production. The addition of CER-001, both at 50 and 500 μg/m, in culture media of LPS-activated PBMCs reverted LPS effects. Also, FACS showed a strong upregulation of CD14 surface expression by PCMBs 24 h following LPS stimulation (FIG. 16). PBMCs treated with LPS and CER-001 in combination maintained CD14 expression at basal level (FIG. 17).

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

Any discussion of documents, acts, materials, devices, articles or the like that has been included in this specification is solely for the purpose of providing a context for the present disclosure. It is not to be taken as an admission that any or all of these matters form part of the prior art base or were common general knowledge in the field relevant to the present disclosure as it existed anywhere before the priority date of this application.

### 8. SPECIFIC EMBODIMENTS

Various aspects of the present disclosure are described in the embodiments set forth in the following numbered paragraphs of Group 1.

### Group 1:

1. A method of treating a subject having or at risk of developing leukocytosis, comprising administering to the subject a dose of a lipid binding protein-based complex effective to reduce the subject's white blood cell count.

2. The method of embodiment 1, wherein the subject has one or more symptoms associated with leukocytosis.

3. A method of treating a subject having one or more symptoms associated with leukocytosis, comprising administering to the subject a dose of a lipid binding protein-based complex effective to reduce the subject's white blood cell count and/or ameliorate one or more of the one or more symptoms associated with leukocytosis.

4. The method of embodiment 2 or embodiment 3, wherein the one or more symptoms comprise a fever.

5. The method of any one of embodiments 2 to 4, wherein the one or more symptoms comprise bleeding or bruising.

6. The method of any one of embodiments 2 to 5, wherein the one or more symptoms comprise sweating.

7. The method of any one of embodiments 2 to 6, wherein the one or more symptoms comprise pain or tingling in the legs, arms, or abdomen.

8. The method of any one of embodiments 2 to 7, wherein the one or more symptoms comprise one or more vision problems.

9. The method of embodiment 8, wherein one or more vision problems comprise blurred vision.

10. The method of embodiment 8 or embodiment 9, wherein the one or more vision problems comprise double vision.

11. The method of any one of embodiments 8 to 10, wherein the one or more vision problems comprise blind spots.

12. The method of any one of embodiments 8 to 11, wherein the one or more vision problems comprise cloudy vision.

13. The method of any one of embodiments 8 to 12, wherein the one or more vision problems comprise dimmed vision.

14. The method of any one of embodiments 2 to 13, wherein the one or more symptoms comprise unclear thinking.

15. The method of any one of embodiments 2 to 14, wherein the one or more symptoms comprise loss of appetite.

16. The method of any one of embodiments 2 to 15, wherein the one or more symptoms comprise trouble breathing (e.g., shortness of breath, below normal blood oxygen levels).

17. The method of any one of embodiments 2 to 16, wherein the dose of a lipid binding protein-based complex is effective to ameliorate one or more of the one or more symptoms associated with leukocytosis.

18. The method of any one of embodiments 1 to 17, wherein the subject has or is at risk of developing leukocytosis due to inflammation, an infection, a white blood cell disorder, physical stress, emotional stress, medication, or an allergic reaction.

19. The method of embodiment 18, wherein the subject has or is at risk of developing leukocytosis due to inflammation.

20. The method of embodiment 18, wherein the subject has or is at risk of developing leukocytosis due to an infection.

21. The method of embodiment 18, wherein the subject has or is at risk of developing leukocytosis due to a white blood cell disorder.

22. The method of embodiment 18, wherein the subject has or is at risk of developing leukocytosis due to physical stress, optionally where the physical stress is from a seizure, anesthesia or overexertion.

23. The method of embodiment 18, wherein the subject has or is at risk of developing leukocytosis due to emotional stress.

24. The method of embodiment 18, wherein the subject has or is at risk of developing leukocytosis due to medication, optionally wherein the medication is a steroid, corticosteroid, lithium, or a beta agonist.

25. The method of embodiment 18, wherein the subject has or is at risk of developing leukocytosis due to an allergic reaction.

26. The method of any one of embodiments 1 to 25, wherein the subject has an infection.

27. The method of embodiment 26, wherein the infection is a viral infection, optionally wherein the viral infection is a coronavirus infection, which is optionally COVID-19.

28. The method of embodiment 26, wherein the infection is a bacterial infection.

29. The method of embodiment 26, wherein the infection is a fungal infection.

30. The method of embodiment 26, wherein the infection is a parasitic infection.

31. The method of any one of embodiments 1 to 30, wherein the subject has diabetes.

32. The method of any one of embodiments 1 to 31, wherein the subject has a white blood cell disorder.

33. The method of embodiment 32, wherein the white blood cell disorder is a primary bone marrow disorder (e.g., an acute leukemia, chronic leukemia, or myeloproliferative disorder).

34. The method of embodiment 32, wherein the white blood cell disorder is neutrophilia, optionally wherein the neutrophilia is idiopathic neutrophilia, which is optionally chronic idiopathic neutrophilia.

35. The method of embodiment 32, wherein the white blood cell disorder is hemolytic anemia.

36. The method of embodiment 32, wherein the white blood cell disorder is thrombocytopenia, optionally wherein the thrombocytopenia is idiopathic thrombocytopenia or essential thrombocytopenia.

37. The method of embodiment 32, wherein the white blood cell disorder is a cancer, which is optionally a bone cancer or blood cancer.

38. The method of embodiment 32, wherein the white blood cell disorder is a myeloproliferative disorder or blood cancer.

39. The method of embodiment 38, wherein the white blood cell disorder is a myeloproliferative disorder.

40. The method of embodiment 38, wherein the white blood cell disorder is a blood cancer.

41. The method of embodiment 38, wherein the white blood cell disorder is lymphoma.

42. The method of embodiment 38, wherein the white blood cell disorder is a leukemia.

43. The method of embodiment 42, wherein the leukemia is a chronic leukemia.

44. The method of embodiment 42, wherein the leukemia is an acute leukemia.

45. The method of embodiment 42, wherein the leukemia is lymphocytic leukemia, chronic eosinophilic leukemia, chronic myelogenous leukemia, or chronic neutrophilic leukemia.

46. The method of embodiment 38, wherein the white blood cell disorder is polycythemia vera.

47. The method of embodiment 38, wherein the white blood cell disorder is myelofibrosis, optionally wherein the myelofibrosis is primary myelofibrosis.

48. The method of any one of embodiments 1 to 47, wherein the subject has leukocytosis.

49. The method of embodiment 48, wherein the subject has a white blood cell count above 11×10⁹ per L.

50. The method of embodiment 48 or embodiment 49, wherein the subject has a white blood cell count above 12×10¹ per L.

51. The method of any one of embodiments 48 to 50, wherein the subject has a white blood cell count above 15×10¹ per L.

52. The method of any one of embodiments 48 to 51, wherein the subject has a white blood cell count above 20×10¹ per L.

53. The method of embodiment 48 or embodiment 49, wherein the subject has a white blood cell count below 100×10⁹ per L.

54. The method of any one of embodiments 48 to 52, wherein the subject has a white blood cell count below 100×10⁹ per L.

55. The method of any one of embodiments 48 to 54, wherein the subject has a white blood cell count below 50×10⁹ per L.

56. The method of any one of embodiments 48 to 55, wherein the subject has a white blood cell count below 25×10⁹ per L.

57. The method of any one of embodiments 48 to 56, wherein the subject has a white blood cell count below 15×10⁹ per L.

58. The method of any one of embodiments 48 to 57, wherein the subject has a white blood cell count between 11×10⁹ per L and 50×10⁹ per L.

59. The method of any one of embodiments 48 to 57, wherein the subject has a white blood cell count between 11×10⁹ per L and 25×10⁹ per L.

60. The method of any one of embodiments 48 to 57, wherein the subject has a white blood cell count between 25×10⁹ per L and 50×10⁹ per L.

61. The method of any one of embodiments 48 to 51, wherein the subject has a white blood cell count between 50×10⁹ per L and 100×10⁹ per L.

62. The method of any one of embodiments 48 to 57, wherein the subject has a white blood cell count between 11×10⁹ per L and 15×10⁹ per L.

63. The method of any one of embodiments 48 to 57, wherein the subject has a white blood cell count between 15×10⁹ per L and 20×10⁹ per L.

64. The method of any one of embodiments 1 to 47, wherein the subject is at risk of leukocytosis.

65. A method of treating a subject having endothelial dysfunction, comprising administering to the subject a dose of a lipid binding protein-based complex, wherein the dose is a high dose.

66. A method of treating a subject experiencing acute coronary syndrome or stroke or who has experienced acute coronary syndrome or stroke, comprising administering to the subject a dose of a lipid binding protein-based complex, wherein the dose is a high dose.

67. The method of embodiment 65 or embodiment 66, wherein the subject is experiencing acute coronary syndrome, optionally wherein the acute coronary syndrome is myocardial infarction (for example ST-elevation myocardial infarction or non-ST elevation myocardial infarction) or unstable angina.

68. The method of embodiment 65 or embodiment 66, wherein the subject has experienced acute coronary syndrome, optionally wherein the acute coronary syndrome is myocardial infarction (for example ST-elevation myocardial infarction or non-ST elevation myocardial infarction) or unstable angina.

69. The method of embodiment 65 or embodiment 66, wherein the subject is experiencing a stroke.

70. The method of embodiment 65 or embodiment 66, wherein the subject has experienced a stroke.

71. A method of treating a subject having or at risk of developing carditis, comprising administering to the subject a dose of a lipid binding protein-based complex, optionally wherein the carditis is myocarditis and/or pericarditis.

72. The method of embodiment 71, wherein the carditis comprises myocarditis.

73. The method of embodiment 71 or embodiment 72, wherein the carditis comprises pericarditis.

74. The method of any one of embodiments 71 to 73, wherein the subject is male.

75. The method of any one of embodiments 71 to 74, wherein the subject is less than 60 years old.

76. The method of any one of embodiments 71 to 74, wherein the subject is less than 50 years old.

77. The method of any one of embodiments 71 to 74, wherein the subject is less than 40 years old.

78. The method of any one of embodiments 71 to 74, wherein the subject is less than 30 years old.

79. The method of any one of embodiments 71 to 74, wherein the subject is less than 20 years old.

80. The method of any one of embodiments 71 to 79, wherein the subject is at least 5 years old.

81. The method of any one of embodiments 71 to 79, wherein the subject is at least 8 years old.

82. The method of any one of embodiments 71 to 79, wherein the subject is at least 10 years old.

83. The method of any one of embodiments 71 to 79, wherein the subject is at least 12 years old.

84. The method of any one of embodiments 71 to 79, wherein the subject is at least 15 years old.

85. The method of any one of embodiments 71 to 79, wherein the subject is at least 18 years old.

86. The method of any one of embodiments 71 to 85, wherein the subject has an infection.

87. The method of embodiment 74, wherein the infection is a viral infection, which is optionally a coronavirus infection, which is optionally COVID-19.

88. The method of embodiment 74, wherein the infection is a bacterial infection.

89. The method of any one of embodiments 71 to 88, wherein the lipid binding protein-based complex is administered before a vaccine is administered to the subject.

90. The method of any one of embodiments 71 to 88, wherein the lipid binding protein-based complex is administered to the subject concurrently with a vaccine.

91. The method of any one of embodiments 71 to 88, wherein the lipid binding protein-based complex is administered after a vaccine is administered to the subject.

92. The method of any one of embodiments 89 to 91, wherein the vaccine is a coronavirus vaccine, which is optionally a COVID-19 vaccine.

93. The method of any one of embodiments 71 to 92, wherein the subject has carditis.

94. The method of embodiment 93, wherein the dose of the lipid binding protein-based complex is effective to ameliorate one or more symptoms of the carditis.

95. The method of any one of embodiments 71 to 92, wherein the subject is at risk of carditis.

96. The method of embodiment 95, wherein the dose of the lipid binding protein-based complex is effective to prevent the carditis or reduce the severity of the carditis.

97. The method of any one of embodiments 1 to 96, wherein the dose is effective to reduce the subject's circulating VCAM-1 and/or ICAM-1.

98. The method of embodiment 97, wherein the dose is effective to reduce the subject's circulating VCAM-1 by at least 100 ng/mL within two days of the first administration of the lipid binding protein-based complex.

99. The method of embodiment 97, wherein the dose is effective to reduce the subject's circulating VCAM-1 by at least 200 ng/mL within two days of the first administration of the lipid binding protein-based complex.

100. The method of embodiment 97, wherein the dose is effective to reduce the subject's circulating VCAM-1 by at least 300 ng/mL within two days of the first administration of the lipid binding protein-based complex.

101. The method of any one of embodiments 97 to 100, wherein the dose is effective to reduce the subject's circulating VCAM-1 by up to 400 ng/mL within two days of the first administration of the lipid binding protein-based complex.

102. The method of embodiment 97 to 101, wherein the dose is effective to reduce the subject's circulating VCAM-1 by at least 100 ng/mL within five days of the first administration of the lipid binding protein-based complex.

103. The method of embodiment 97 to 101, wherein the dose is effective to reduce the subject's circulating VCAM-1 by at least 200 ng/mL within five days of the first administration of the lipid binding protein-based complex.

104. The method of embodiment 97 to 101, wherein the dose is effective to reduce the subject's circulating VCAM-1 by at least 300 ng/mL within five days of the first administration of the lipid binding protein-based complex.

105. The method of any one of embodiments 97 to 104, wherein the dose is effective to reduce the subject's circulating VCAM-1 by up to 400 ng/mL within five days of the first administration of the lipid binding protein-based complex.

106. The method of any one of embodiments 97 to 105, wherein the dose is effective to reduce the subject's circulating ICAM-1 by at least 50 ng/mL within two days of the first administration of the lipid binding protein-based complex.

107. The method of any one of embodiments 97 to 105, wherein the dose is effective to reduce the subject's circulating ICAM-1 by at least 75 ng/mL within two days of the first administration of the lipid binding protein-based complex.

108. The method of any one of embodiments 97 to 105, wherein the dose is effective to reduce the subject's circulating ICAM-1 by at least 100 ng/mL within two days of the first administration of the lipid binding protein-based complex.

10⁹. The method of any one of embodiments 97 to 108, wherein the dose is effective to reduce the subject's circulating ICAM-1 by up to 125 ng/mL within two days of the first administration of the lipid binding protein-based complex.

110. The method of any one of embodiments 97 to 110, wherein the dose is effective to reduce the subject's circulating ICAM-1 by at least 50 ng/mL within five days of the first administration of the lipid binding protein-based complex.

111. The method of any one of embodiments 97 to 110, wherein the dose is effective to reduce the subject's circulating ICAM-1 by at least 75 ng/mL within five days of the first administration of the lipid binding protein-based complex.

112. The method of any one of embodiments 97 to 110, wherein the dose is effective to reduce the subject's circulating ICAM-1 by at least 100 ng/mL within five days of the first administration of the lipid binding protein-based complex.

113. The method of any one of embodiments 97 to 112, wherein the dose is effective to reduce the subject's circulating ICAM-1 by up to 125 ng/mL within five days of the first administration of the lipid binding protein-based complex.

114. The method of any one of embodiments 1 to 113, wherein the dose is a high dose.

115. The method of embodiment 114, wherein the high dose is administered over a period of two days to approximately two weeks, optionally wherein the high dose is administered over a period of two days, three days, four days, five days, six days, seven days, eight days, nine days, 10 days, eleven days, 12 days, 13 days, 14 days or 15 days.

116. The method of embodiment 115, wherein the high dose is administered over a period of two days.

117. The method of embodiment 115, wherein the high dose is administered over a period of three days.

118. The method of embodiment 115, wherein the high dose is administered over a period of four days.

119. The method of embodiment 115, wherein the high dose is administered over a period of five days.

120. The method of embodiment 115, wherein the high dose is administered over a period of six days.

121. The method of embodiment 115, wherein the high dose is administered over a period of seven days.

122. The method of embodiment 115, wherein the high dose is administered over a period of eight days.

123. The method of embodiment 115, wherein the high dose is administered over a period of nine days.

124. The method of embodiment 115, wherein the high dose is administered over a period of 10 days.

125. The method of embodiment 115, wherein the high dose is administered over a period of 11 days.

126. The method of embodiment 115, wherein the high dose is administered over a period of 12 days.

127. The method of embodiment 115, wherein the high dose is administered over a period of 13 days.

128. The method of embodiment 115, wherein the high dose is administered over a period of 14 days.

129. The method of embodiment 115, wherein the high dose is administered over a period of 15 days.

130. The method of any one of embodiments 114 to 129, wherein the high dose is the aggregate of two to 20 individual doses, optionally wherein the high dose is an aggregate of two, three, four, five, six, seven, eight, nine or 10 individual doses.

131. The method of embodiment 130, wherein the high dose comprises two to 16 individual doses.

132. The method of embodiment 130, wherein the high dose comprises two to 12 individual doses.

133. The method of embodiment 130, wherein the high dose comprises two to 10 individual doses.

134. The method of embodiment 130, wherein the high dose comprises two to eight individual doses.

135. The method of embodiment 130, wherein the high dose comprises two to six individual doses.

136. The method of embodiment 130, wherein the high dose comprises two to four individual doses.

137. The method of embodiment 130, wherein the high dose comprises four to 20 individual doses.

138. The method of embodiment 130, wherein the high dose comprises four to 16 individual doses.

139. The method of embodiment 130, wherein the high dose comprises four to ten individual doses.

140. The method of embodiment 130, wherein the high dose comprises four to six individual doses.

141. The method of embodiment 130, wherein the high dose comprises six to 20 individual doses.

142. The method of embodiment 130, wherein the high dose comprises six to 16 individual doses.

143. The method of embodiment 130, wherein the high dose comprises six to ten individual doses.

144. The method of embodiment 130, wherein the high dose comprises six to eight individual doses.

145. The method of embodiment 130, wherein the high dose comprises eight to 20 individual doses.

146. The method of embodiment 130, wherein the high dose comprises eight to 16 individual doses.

147. The method of embodiment 130, wherein the high dose comprises eight to ten individual doses.

148. The method of embodiment 130, wherein the high dose comprises 10 to 20 individual doses.

149. The method of embodiment 130, wherein the high dose comprises 10 to 16 individual doses.

150. The method of embodiment 130, wherein the high dose comprises 16 to 20 individual doses.

151. The method of embodiment 130, wherein the high dose comprises two individual doses.

152. The method of embodiment 130, wherein the high dose comprises three individual doses.

153. The method of embodiment 130, wherein the high dose comprises four individual doses.

154. The method of embodiment 130, wherein the high dose comprises five individual doses.

155. The method of embodiment 130, wherein the high dose comprises six individual doses.

156. The method of embodiment 130, wherein the high dose comprises seven individual doses.

157. The method of embodiment 130, wherein the high dose comprises eight individual doses.

158. The method of embodiment 130, wherein the high dose comprises nine individual doses.

159. The method of embodiment 130, wherein the high dose comprises ten individual doses.

160. The method of any one of embodiments 130 to 159, wherein a plurality of individual doses are administered daily or twice daily.

161. The method of any one of embodiments 130 to 159, wherein a plurality of individual doses are administered approximately 12 hours apart.

162. The method of any one of embodiments 130 to 161, wherein a plurality of individual doses are administered two to three days apart.

163. The method of any one of embodiments 130 to 162, wherein each individual dose is effective to increase the subject's HDL levels.

164. The method of embodiment 163, wherein each individual dose is effective to increase the subject's HDL levels by at least 25%, at least 30% or at least 35% 2-4 hours after administration.

165. The method of embodiment 164, wherein each individual dose is effective to increase the subject's HDL levels by at least 25%, at least 30% or at least 35% 2 hours after administration.

166. The method of embodiment 164, wherein each individual dose is effective to increase the subject's HDL levels by at least 25%, at least 30% or at least 35% 3 hours after administration.

167. The method of embodiment 164, wherein each individual dose is effective to increase the subject's HDL levels by at least 25%, at least 30% or at least 35% 4 hours after administration.

168. The method of any one of embodiments 130 to 167, wherein each individual dose is effective to increase the subject's ApoA-I levels.

169. The method of embodiment 168, wherein each individual dose is effective to increase the subject's ApoA-I levels by at least 25%, at least 30% or at least 35% 2-4 hours after administration.

170. The method of embodiment 169, wherein each individual dose is effective to increase the subject's ApoA-I levels by at least 25%, at least 30% or at least 35% 2 hours after administration.

171. The method of embodiment 169, wherein each individual dose is effective to increase the subject's ApoA-I levels by at least 25%, at least 30% or at least 35% 3 hours after administration.

172. The method of embodiment 169, wherein each individual dose is effective to increase the subject's ApoA-I levels by at least 25%, at least 30% or at least 35% 4 hours after administration.

173. The method of any one of embodiments 1 to 172, wherein the dose is effective to improve the subject's vascular endothelial function, optionally wherein vascular endothelial function is measured by circulating VCAM-1 and/or ICAM-1.

174. The method of embodiment 173, wherein the dose is effective to reduce the subject's circulating VCAM-1 by at least 100 ng/mL within two days of the first administration of the lipid binding protein-based complex.

175. The method of embodiment 173, wherein the dose is effective to reduce the subject's circulating VCAM-1 by at least 200 ng/mL within two days of the first administration of the lipid binding protein-based complex.

176. The method of embodiment 173, wherein the dose is effective to reduce the subject's circulating VCAM-1 by at least 300 ng/mL within two days of the first administration of the lipid binding protein-based complex.

177. The method of any one of embodiments 173 to 176, wherein the dose is effective to reduce the subject's circulating VCAM-1 by up to 400 ng/mL within two days of the first administration of the lipid binding protein-based complex.

178. The method of embodiment 173 to 177, wherein the dose is effective to reduce the subject's circulating VCAM-1 by at least 100 ng/mL within five days of the first administration of the lipid binding protein-based complex.

179. The method of embodiment 173 to 177, wherein the dose is effective to reduce the subject's circulating VCAM-1 by at least 200 ng/mL within five days of the first administration of the lipid binding protein-based complex.

180. The method of embodiment 173 to 177, wherein the dose is effective to reduce the subject's circulating VCAM-1 by at least 300 ng/mL within five days of the first administration of the lipid binding protein-based complex.

181. The method of any one of embodiments 173 to 180, wherein the dose is effective to reduce the subject's circulating VCAM-1 by up to 400 ng/mL within five days of the first administration of the lipid binding protein-based complex.

182. The method of any one of embodiments 173 to 181, wherein the dose is effective to reduce the subject's circulating ICAM-1 by at least 50 ng/mL within two days of the first administration of the lipid binding protein-based complex.

183. The method of any one of embodiments 173 to 181, wherein the dose is effective to reduce the subject's circulating ICAM-1 by at least 75 ng/mL within two days of the first administration of the lipid binding protein-based complex.

184. The method of any one of embodiments 173 to 181, wherein the dose is effective to reduce the subject's circulating ICAM-1 by at least 100 ng/mL within two days of the first administration of the lipid binding protein-based complex.

185. The method of any one of embodiments 173 to 184, wherein the dose is effective to reduce the subject's circulating ICAM-1 by up to 125 ng/mL within two days of the first administration of the lipid binding protein-based complex.

186. The method of any one of embodiments 173 to 185, wherein the dose is effective to reduce the subject's circulating ICAM-1 by at least 50 ng/mL within five days of the first administration of the lipid binding protein-based complex.

187. The method of any one of embodiments 173 to 185, wherein the dose is effective to reduce the subject's circulating ICAM-1 by at least 75 ng/mL within five days of the first administration of the lipid binding protein-based complex.

188. The method of any one of embodiments 173 to 185, wherein the dose is effective to reduce the subject's circulating ICAM-1 by at least 100 ng/mL within five days of the first administration of the lipid binding protein-based complex.

189. The method of any one of embodiments 173 to 188, wherein the dose is effective to reduce the subject's circulating ICAM-1 by up to 125 ng/mL within five days of the first administration of the lipid binding protein-based complex.

190. The method of any one of embodiments 1 to 189, wherein the dose is effective to reduce the subject's white blood cell count by at least 2000 WBCs per microliter within two days of the first administration of the lipid binding protein-based complex.

191. The method of any one of embodiments 1 to 189, wherein the dose is effective to reduce the subject's white blood cell count by at least 3000 WBCs per microliter within two days of the first administration of the lipid binding protein-based complex.

192. The method of any one of embodiments 1 to 189, wherein the dose is effective to reduce the subject's white blood cell count by at least 4000 WBCs per microliter within two days of the first administration of the lipid binding protein-based complex.

193. The method of any one of embodiments 1 to 189, wherein the dose is effective to reduce the subject's white blood cell count by at least 5000 WBCs per microliter within two days of the first administration of the lipid binding protein-based complex.

194. The method of any one of embodiments 1 to 189, wherein the dose is effective to reduce the subject's white blood cell count by at least 6000 WBCs per microliter within two days of the first administration of the lipid binding protein-based complex.

195. The method of any one of embodiments 1 to 194, wherein the dose is effective to reduce the subject's white blood cell count by up to 8000 WBCs per microliter within two days of the first administration of the lipid binding protein-based complex.

196. The method of any one of embodiments 1 to 189, wherein the dose is effective to reduce the subject's white blood cell count by at least 2000 WBCs per microliter within five days of the first administration of the lipid binding protein-based complex.

197. The method of any one of embodiments 1 to 189, wherein the dose is effective to reduce the subject's white blood cell count by at least 3000 WBCs per microliter within five days of the first administration of the lipid binding protein-based complex.

198. The method of any one of embodiments 1 to 189, wherein the dose is effective to reduce the subject's white blood cell count by at least 4000 WBCs per microliter within five days of the first administration of the lipid binding protein-based complex.

199. The method of any one of embodiments 1 to 189, wherein the dose is effective to reduce the subject's white blood cell count by at least 5000 WBCs per microliter within five days of the first administration of the lipid binding protein-based complex.

200. The method of any one of embodiments 196 to 199, wherein the dose is effective to reduce the subject's white blood cell count by up to 6000 WBCs per microliter within two days of the first administration of the lipid binding protein-based complex.

201. The method of any one of embodiments 1 to 200, wherein the dose is effective to reduce serum levels of one or more inflammatory markers in the subject.

202. The method of embodiment 201, wherein the dose is effective to reduce serum levels of interleukin-6 (“IL-6”).

203. The method of embodiment 202, wherein the dose is effective to reduce serum levels of IL-6 by at least 20 μg/mL within two days of the first administration of the lipid binding protein-based complex.

204. The method of embodiment 202, wherein the dose is effective to reduce serum levels of IL-6 by at least 30 μg/mL within two days of the first administration of the lipid binding protein-based complex.

205. The method of any one of embodiments 202 to 204, wherein the dose is effective to reduce serum levels of IL-6 by up to 40 μg/mL within two days of the first administration of the lipid binding protein-based complex.

206. The method of any one of embodiments 202 to 205, wherein the dose is effective to reduce serum levels of IL-6 by at least 20 μg/mL within five days of the first administration of the lipid binding protein-based complex.

207. The method of any one of embodiments 202 to 205, wherein the dose is effective to reduce serum levels of IL-6 by at least 30 μg/mL within five days of the first administration of the lipid binding protein-based complex.

208. The method of any one of embodiments 202 to 205, wherein the dose is effective to reduce serum levels of IL-6 by at least 40 μg/mL within five days of the first administration of the lipid binding protein-based complex.

209. The method of any one of embodiments 202 to 208, wherein the dose is effective to reduce serum levels of IL-6 by up to 60 μg/mL within five days of the first administration of the lipid binding protein-based complex.

210. The method of any one of embodiments 202 to 208, wherein the dose is effective to reduce serum levels of IL-6 by up to 50 μg/mL within five days of the first administration of the lipid binding protein-based complex.

211. The method of any one of embodiments 201 to 210, wherein the dose is effective to reduce serum levels of C-reactive protein.

212. The method of any one of embodiments 201 to 211, wherein the dose is effective to reduce serum levels of D-dimer.

213. The method of any one of embodiments 201 to 212, wherein the dose is effective to reduce serum levels of ferritin.

214. The method of embodiment 213, wherein the dose is effective to reduce serum levels of ferritin by at least 200 ng/mL within two days of the first administration of the lipid binding protein-based complex.

215. The method of embodiment 213, wherein the dose is effective to reduce serum levels of ferritin by at least 300 ng/mL within two days of the first administration of the lipid binding protein-based complex.

216. The method of embodiment 213, wherein the dose is effective to reduce serum levels of ferritin by at least 400 ng/mL within two days of the first administration of the lipid binding protein-based complex.

217. The method of any one of embodiments 213 to 216, wherein the dose is effective to reduce serum levels of ferritin by up to 700 ng/mL within two days of the first administration of the lipid binding protein-based complex.

218. The method of any one of embodiments 213 to 217, wherein the dose is effective to reduce serum levels of ferritin by at least 200 ng/mL within five days of the first administration of the lipid binding protein-based complex.

219. The method of any one of embodiments 213 to 217, wherein the dose is effective to reduce serum levels of ferritin by at least 300 ng/mL within five days of the first administration of the lipid binding protein-based complex.

220. The method of any one of embodiments 213 to 217, wherein the dose is effective to reduce serum levels of ferritin by at least 400 ng/mL within five days of the first administration of the lipid binding protein-based complex.

221. The method of any one of embodiments 213 to 220, wherein the dose is effective to reduce serum levels of ferritin by up to 700 ng/mL within two days of the first administration of the lipid binding protein-based complex.

222. The method of any one of embodiments 201 to 221, wherein the dose is effective to reduce serum levels of interleukin 8 (IL-8).

223. The method of embodiment 222, wherein the dose is effective to reduce serum levels of IL-8 by at least 100 μg/mL within two days of the first administration of the lipid binding protein-based complex.

224. The method of embodiment 222, wherein the dose is effective to reduce serum levels of IL-8 by at least 150 μg/mL within two days of the first administration of the lipid binding protein-based complex.

225. The method of any one of embodiments 222 to 224, wherein the dose is effective to reduce serum levels of IL-8 by up to 300 μg/mL within two days of the first administration of the lipid binding protein-based complex.

226. The method of any one of embodiments 222 to 225, wherein the dose is effective to reduce serum levels of IL-8 by at least 100 μg/mL within five days of the first administration of the lipid binding protein-based complex.

227. The method of any one of embodiments 222 to 225, wherein the dose is effective to reduce serum levels of IL-8 by at least 150 μg/mL within five days of the first administration of the lipid binding protein-based complex.

228. The method of any one of embodiments 222 to 227, wherein the dose is effective to reduce serum levels of IL-8 by up to 300 μg/mL within five days of the first administration of the lipid binding protein-based complex.

229. The method of any one of embodiments 201 to 228, wherein the dose is effective to reduce serum levels of granulocyte-macrophage colony stimulating factor (GM-CSF).

230. The method of any one of embodiments 201 to 229, wherein the dose is effective to reduce serum levels of monocyte chemoattractant protein (MCP) 1.

231. The method of any one of embodiments 201 to 230, wherein the dose is effective to reduce serum levels of tumor necrosis factor α (TNF-α).

232. The method of any one of embodiments 201 to 231, wherein the dose is effective to reduce serum levels of KIM-1.

233. The method of any one of embodiments 201 to 232, wherein the dose is effective to reduce serum levels of the one or more inflammatory markers from an elevated range to a normal range.

234. The method of any one of embodiments 201 to 233, wherein the dose is effective to reduce serum levels of the one or more inflammatory markers by at least 20%, by at least 40% or by at least 60%.

235. The method of any one of embodiments 1 to 234, wherein the subject has an endotoxin activity level of >0.6 prior to administration of the lipid binding protein-based complex.

236. The method of any one of embodiments 1 to 235, wherein the dose is effective to reduce the subject's endotoxin activity level.

237. The method of any one of embodiments 1 to 236, wherein the dose is effective to transiently increase serum triglyceride levels.

238. The method of embodiment 237, wherein the transiently increase is an increase for up to 9 days.

239. The method of any one of embodiments 1 to 238, wherein the lipid binding protein-based complex is a reconstituted HDL or HDL mimetic.

240. The method of any one of embodiments 1 to 238, wherein the lipid binding protein-based complex is an Apomer or a Cargomer.

241. The method of any one of embodiments 1 to 240, wherein the lipid binding protein-based complex comprises a sphingomyelin.

242. The method of any one of embodiments 1 to 241, wherein the lipid binding protein-based complex comprises a negatively charged lipid.

243. The method of embodiment 242, wherein the negatively charged lipid is 1,2-dipalmitoyl-sn-glycero-3-[phospho-rac-(1-glycerol) (DPPG) or a salt thereof.

244. The method of embodiment 239, wherein the lipid binding protein-based complex is CER-001, CSL-111, CSL-112, CER-522 or ETC-216.

245. The method of embodiment 244, wherein the lipid binding protein-based complex is CER-001.

246. The method of any one of embodiments 1 to 245, wherein the lipid binding protein-based complex is administered systemically, optionally by infusion.

247. The method of any one of embodiments 1 to 246, wherein the lipid binding protein-based complex is administered until serum levels of one or more inflammatory markers are reduced.

248. The method of embodiment 247, wherein the lipid binding protein-based complex is administered until serum levels of one or more inflammatory markers are reduced to a normal range(s).

249. The method of embodiment 247, wherein the lipid binding protein-based complex is administered until serum levels of one or more inflammatory markers are reduced below a baseline level(s) for the one or more inflammatory markers measured prior to lipid binding protein-based complex administration.

250. The method of any one of embodiments 1 to 249, wherein each individual dose of the lipid binding protein-based complex administered is 4-40 mg/kg (on a protein weight basis).

251. The method of embodiment 250, wherein each individual dose of the lipid binding protein-based complex is 4-30 mg/kg (on a protein weight basis).

252. The method of embodiment 250, wherein each individual dose of the lipid binding protein-based complex is 15-25 mg/kg (on a protein weight basis).

253. The method of embodiment 250, wherein each individual dose of the lipid binding protein-based complex is 10-30 mg/kg (on a protein weight basis).

254. The method of embodiment 250, wherein each individual dose of the lipid binding protein-based complex is 10-20 mg/kg (on a protein weight basis).

255. The method of embodiment 250, wherein each individual dose of the lipid binding protein-based complex is 5 mg/kg (on a protein weight basis).

256. The method of embodiment 250, wherein each individual dose of the lipid binding protein-based complex is 10 mg/kg (on a protein weight basis).

257. The method of embodiment 250, wherein each individual dose of the lipid binding protein-based complex is 15 mg/kg (on a protein weight basis).

258. The method of embodiment 250, wherein each individual dose of the lipid binding protein-based complex is 20 mg/kg (on a protein weight basis).

259. The method of embodiment 250, wherein each individual dose of the lipid binding protein-based complex is 5 to 15 mg/kg (on a protein weight basis).

260. The method of embodiment 250, wherein each individual dose of the lipid binding protein-based complex is 10 to 20 mg/kg (on a protein weight basis).

261. The method of embodiment 250, wherein each individual dose of the lipid binding protein-based complex is 15 to 25 mg/kg (on a protein weight basis).

262. The method of any one of embodiments 1 to 261, wherein the dose is administered according to an induction regimen, optionally followed by a consolidation regimen.

263. The method of embodiment 262, wherein the induction regimen comprises administering the lipid binding protein-based complex once daily or twice daily.

264. The method of embodiment 262 or embodiment 263, wherein the consolidation regimen comprises administering the lipid binding protein-based complex once daily or once every two days.

265. The method of any one of embodiments 1 to 264, wherein the subject is not treated with a maintenance regimen.

266. The method of any one of embodiments embodiment 262 to 265, wherein the consolidation regimen comprises administering one or more doses of the lipid binding protein-based complex to the subject one or more days after administration of the final dose of the induction regimen.

267. The method of embodiment 266, wherein the first dose of the lipid binding protein-based complex administered during the consolidation regimen is administered two or more days after administration of the final dose of the induction regimen.

268. The method of embodiment 266, wherein the first dose of the lipid binding protein-based complex administered during the consolidation regimen is administered three or more days after administration of the final dose of the induction regimen.

269. The method of embodiment 268, wherein the first dose of the lipid binding protein-based complex administered during the consolidation regimen is administered three days after administration of the final dose of the induction regimen.

270. The method of any one of embodiments 262 to 269, which comprises an induction regimen comprising twice daily administration of the lipid binding protein-based complex on days 1, 2, and 3 and a consolidation regimen comprising two doses of the lipid binding protein-based complex on day 6.

271. The method of any one of embodiments 262 to 270, wherein each individual dose of the lipid binding protein-based complex administered in the induction regimen is 4-40 mg/kg (on a protein weight basis).

272. The method of any one of embodiments 262 to 271, wherein each individual dose of the lipid binding protein-based complex administered in the induction regimen is 4-30 mg/kg (on a protein weight basis).

273. The method of any one of embodiments 262 to 271, wherein each individual dose of the lipid binding protein-based complex administered in the induction regimen is 15-25 mg/kg (on a protein weight basis).

274. The method of any one of embodiments 262 to 271, wherein each individual dose of the lipid binding protein-based complex administered in the induction regimen is 10-30 mg/kg (on a protein weight basis).

275. The method of any one of embodiments 262 to 271, wherein each individual dose of the lipid binding protein-based complex administered in the induction regimen is 10-20 mg/kg (on a protein weight basis).

276. The method of any one of embodiments 262 to 271, wherein each individual dose of the lipid binding protein-based complex administered in the induction regimen is 5 mg/kg (on a protein weight basis).

277. The method of any one of embodiments 262 to 271, wherein each individual dose of the lipid binding protein-based complex administered in the induction regimen is 10 mg/kg (on a protein weight basis).

278. The method of any one of embodiments 262 to 271, wherein each individual dose of the lipid binding protein-based complex administered in the induction regimen is 15 mg/kg (on a protein weight basis).

279. The method of any one of embodiments 262 to 271, wherein each individual dose of the lipid binding protein-based complex administered in the induction regimen is 20 mg/kg (on a protein weight basis).

280. The method of any one of embodiments 262 to 279, wherein the dose of the lipid binding protein-based complex administered in the consolidation regimen is 5 to 15 mg/kg (on a protein weight basis).

281. The method of any one of embodiments 262 to 279, wherein the dose of the lipid binding protein-based complex administered in the consolidation regimen is 10 to 20 mg/kg (on a protein weight basis).

282. The method of any one of embodiments 262 to 279, wherein the dose of the lipid binding protein-based complex administered in the consolidation regimen is 15 to 25 mg/kg (on a protein weight basis).

283. The method of any one of embodiments 262 to 279, wherein the dose of the lipid binding protein-based complex administered in the consolidation regimen is 5 mg/kg (on a protein weight basis).

284. The method of any one of embodiments 262 to 279, wherein the dose of the lipid binding protein-based complex administered in the consolidation regimen is 10 mg/kg (on a protein weight basis).

285. The method of any one of embodiments 262 to 279, wherein the dose of the lipid binding protein-based complex administered in the consolidation regimen is 15 mg/kg (on a protein weight basis).

286. The method of any one of embodiments 1 to 285, wherein each individual dose of the lipid binding protein-based complex administered is 300 mg to 4000 mg (on a protein weight basis).

287. The method of embodiment 286, wherein each individual dose of the lipid binding protein-based complex administered is 300 mg to 3000 mg (on a protein weight basis).

288. The method of embodiment 286, wherein each individual dose of the lipid binding protein-based complex administered is 300 mg to 1500 mg (on a protein weight basis).

289. The method of embodiment 286, wherein each individual dose of the lipid binding protein-based complex administered is 400 mg to 4000 mg (on a protein weight basis).

290. The method of embodiment 286, wherein each individual dose of the lipid binding protein-based complex administered is 400 mg to 1500 mg (on a protein weight basis).

291. The method of embodiment 286, wherein each individual dose of the lipid binding protein-based complex administered is 500 mg to 1200 mg (on a protein weight basis).

292. The method of embodiment 286, wherein each individual dose of the lipid binding protein-based complex administered is 500 mg to 1000 mg (on a protein weight basis).

293. The method of embodiment 286, wherein each individual dose of the lipid binding protein-based complex administered is 600 mg to 3000 mg (on a protein weight basis).

294. The method of embodiment 286, wherein each individual dose of the lipid binding protein-based complex administered is 800 mg to 3000 mg (on a protein weight basis).

295. The method of embodiment 286, wherein each individual dose of the lipid binding protein-based complex administered is 1000 mg to 2400 mg (on a protein weight basis).

296. The method of embodiment 286, wherein each individual dose of the lipid binding protein-based complex administered is 1000 mg to 2000 mg (on a protein weight basis).

297. The method of any one of embodiments 1 to 296, wherein the high dose of the lipid binding protein-based complex is 600 mg to 40 g (on a protein weight basis).

298. The method of any one of embodiments 1 to 296, wherein the high dose of the lipid binding protein-based complex is 3 g to 35 g (on a protein weight basis).

299. The method of any one of embodiments 1 to 296, wherein the high dose of the lipid binding protein-based complex is 5 g to 30 g (on a protein weight basis).

300. The method of any one of embodiments 1 to 299, wherein the lipid binding protein-based complex is administered by infusion.

301. The method of embodiment 300, wherein each individual dose is administered over a one to 24-hour period.

302. The method of embodiment 301, wherein each individual dose is administered over a 24-hour period.

303. The method of any one of embodiments 1 to 302, which further comprises administering an antihistamine to the subject prior to each individual dose.

304. The method of embodiment 303, wherein the antihistamine comprises dexchlorpheniramine or hydroxyzine.

305. The method of any one of embodiments 1 to 304, wherein the subject is receiving or has received one or more additional therapies and/or which further comprises administering to the subject one or more additional therapies.

306. The method of embodiment 305, wherein the one or more additional therapies comprise a standard of care therapy.

307. The method of embodiment 305 or embodiment 306, wherein the subject has an infection and the one or more additional therapies comprises antibiotic therapy.

308. The method of embodiment 305 or embodiment 306, wherein the one or more additional therapies comprises hemodynamic support.

309. The method of any one of embodiments 305 to 308, wherein the one or more additional therapies comprise one or more anti-IL-6 agents.

310. The method of embodiment 309, wherein the one or more anti-IL-6 agents comprise tocilizumab, siltuximab, olokizumab, elsilimomab, BMS-945429, sirukumab, levilimab, CPSI-2364, or a combination thereof.

311. The method of embodiment 310, wherein the one or more anti-IL-6 agents comprise tocilizumab.

312. The method of any one of embodiments 305 to 311, wherein the one or more additional therapies comprise one or more corticosteroids.

313. The method of embodiment 312, wherein the one or more corticosteroids comprise methylprednisolone, dexamethasone, or a combination thereof.

314. The method of any one of embodiments 305 to 313, wherein the subject has or has had a COVID-19 infection and the one or more additional therapies comprise antibodies from recovered COVID-19 patients.

315. The method of any one of embodiments 305 to 314, wherein the subject has or has had a COVID-19 infection and the one or more additional therapies comprise antibodies against the spike protein of COVID-19.

316. The method of any one of embodiments 305 to 315, wherein the subject has or has had a COVID-19 infection and the one or more additional therapies comprise one or more antiviral agents.

317. The method of embodiment 316, wherein the one or more antiviral agents comprise lopinavir.

318. The method of embodiment 316 or embodiment 317, wherein the one or more antiviral agents comprise remdesivir.

319. The method of any one of embodiments 316 to 318, wherein the one or more antiviral agents comprise danoprevir.

320. The method of any one of embodiments 316 to 319, wherein the one or more antiviral agents comprise galidesivir.

321. The method of any one of embodiments 316 to 320, wherein the one or more antiviral agents comprise darunavir.

322. The method of any one of embodiments 316 to 321, wherein the one or more antiviral agents comprise ritonavir.

323. The method of any one of embodiments 305 to 322, wherein the subject has or has had a COVID-19 infection and the one or more additional therapies comprise chloroquine or hydroxychloroquine.

324. The method of any one of embodiments 305 to 323, wherein the subject has or has had a COVID-19 infection and the one or more additional therapies comprise azithromycin.

325. The method of any one of embodiments 305 to 324, wherein the subject has or has had a COVID-19 infection and the one or more additional therapies comprise an interferon.

326. The method of embodiment 325, wherein the interferon is an interferon alpha.

327. The method of embodiment 325, wherein the interferon is an interferon beta.

328. The method of any one of embodiments 325 to 327, wherein the interferon is pegylated.

329. The method of any one of embodiments 1 to 328, wherein the lipid binding protein-based complex is CER-001.

330. The method of embodiment 329, wherein the CER-001 is a lipoprotein complex comprising ApoA-I and phospholipids in a ApoA-I weight:total phospholipid weight ratio of 1:2.7+/−20% and the phospholipids sphingomyelin and DPPG in a sphingomyelin:DPPG weight:weight ratio of 97:3+/−20%.

331. The method of embodiment 329, wherein the CER-001 is a lipoprotein complex comprising ApoA-I and phospholipids in a ApoA-I weight:total phospholipid weight ratio of 1:2.7+/−10% and the phospholipids sphingomyelin and DPPG in a sphingomyelin:DPPG weight:weight ratio of 97:3+/−10%.

332. The method of embodiment 329, wherein the CER-001 is a lipoprotein complex comprising ApoA-I and phospholipids in a ApoA-I weight:total phospholipid weight ratio of 1:2.7 and the phospholipids sphingomyelin and DPPG in a sphingomyelin:DPPG weight:weight ratio of 97:3.

333. The method of any one of embodiments 330 to 332, wherein the ApoA-I has the amino acid sequence of amino acids 25-267 of SEQ ID NO:2.

334. The method of any one of embodiments 330 to 333, wherein the ApoA-I is recombinantly expressed.

335. The method of any one of embodiments 330 to 334, wherein the CER-001 comprises natural sphingomyelin.

336. The method of embodiment 335, wherein the natural sphingomyelin is chicken egg sphingomyelin.

337. The method of any one of embodiments 330 to 334, wherein the CER-001 comprises synthetic sphingomyelin.

338. The method of embodiment 337, wherein the synthetic sphingomyelin is palmitoylsphingomyelin.

339. The method of any one of embodiments 329 to 338, wherein CER-001 is administered in the form of a formulation in which the CER-001 is at least 95% homogeneous.

340. The method of embodiment 339, wherein CER-001 is administered in the form of a formulation in which the CER-001 is at least 97% homogeneous.

341. The method of embodiment 339, wherein CER-001 is administered in the form of a formulation in which the CER-001 is at least 98% homogeneous.

342. The method of embodiment 339, wherein CER-001 is administered in the form of a formulation in which the CER-001 is at least 99% homogeneous.

Various aspects of the present disclosure are described in the embodiments set forth in the following numbered paragraphs of Group 2.

### Group 2:

1. A method of treating a subject having one or more symptoms associated with leukocytosis, comprising administering to the subject a dose of an Apolipoprotein A-I (“ApoA-I”) formulation comprising ApoA-I and one or more lipids, wherein the ApoA-I and the lipids are in the form of lipoprotein complexes, effective to reduce the subject's white blood cell count and/or ameliorate one or more of the one or more symptoms associated with leukocytosis.

2. A method of treating a subject having endothelial dysfunction, comprising administering to the subject an ApoA-I formulation comprising ApoA-I and one or more lipids, wherein the ApoA-I and the lipids are in the form of lipoprotein complexes.

3. A method of treating a subject having or at risk of developing carditis, comprising administering to the subject an ApoA-I formulation comprising ApoA-I and one or more lipids, wherein the ApoA-I and the lipids are in the form of lipoprotein complexes.

4. A method of treating a subject having or at risk of developing leukocytosis, comprising administering to the subject a dose of an ApoA-I formulation comprising ApoA-I and one or more lipids, wherein the ApoA-I and the lipids are in the form of lipoprotein complexes, effective to reduce the subject's white blood cell count.

5. A method of treating a subject experiencing acute coronary syndrome or stroke or who has experienced acute coronary syndrome or stroke, comprising administering to the subject an ApoA-I formulation comprising ApoA-I and one or more lipids, wherein the ApoA-I and the lipids are in the form of lipoprotein complexes.

6. The method of any one of embodiments 1 to 5, wherein the ApoA-I has the amino acid sequence of amino acids 25-267 of SEQ ID NO:2.

7. The method of any one of embodiments 1 to 6, wherein the ApoA-I is recombinantly expressed.

8. The method of any one of embodiments 1 to 7, wherein said one or more lipids comprise neutral lipids.

9. The method of embodiment 8, wherein the neutral lipids comprise sphingomyelin.

10. The method of embodiment 9, wherein the neutral lipids consist of sphingomyelin.

11. The method of embodiment 9 or embodiment 10, wherein the sphingomyelin comprises natural sphingomyelin.

12. The method of embodiment 11, wherein the natural sphingomyelin is chicken egg sphingomyelin.

13. The method of embodiment 9 or embodiment 10, wherein the sphingomyelin comprises synthetic sphingomyelin.

14. The method of embodiment 13, wherein the synthetic sphingomyelin is palmitoylsphingomyelin.

15. The method of any one of embodiments 1 to 10, wherein the one or more lipids further comprise negatively charged lipids.

16. The method of embodiment 15, wherein the negatively charged lipids comprise 1,2-dipalmitoyl-sn-glycero-3-[phospho-rac-(1-glycerol)](“DPPG”) or a salt thereof.

17. The method of embodiment 16, wherein the negatively charged lipids consist of DPPG or a salt thereof.

18. The method of any one of embodiments 11 to 17, wherein the molar ratio of the components of the negatively charged lipid to the neutral lipid to the ApoA-I in the formulation is 2-6:90-120:1.

19. The method of any one of embodiments 8 to 18, wherein said lipids consist of 95 to 99 weight % neutral phospholipid and 1 to 5 weight % negatively charged phospholipid.

20. The method embodiment 19, wherein said lipids consist of 96 to 98 weight % neutral phospholipid and 2 to 4 weight % negatively charged phospholipid.

21. The method of embodiment 20, wherein said lipids consists of 97 weight % neutral phospholipid and 3 weight % negatively charged phospholipid.

22. The method of any one of embodiments 1 to 21 which has an ApoA-I to lipid ratio ranging from 1:2 to 1:3 by weight.

23. The method of embodiment 22, which has an ApoA-I to lipid ratio of about 1:2.7 by weight.

24. The method of any one of embodiments 1 to 23, wherein the lipoprotein complexes are at least 95% homogeneous as reflected by a single peak in gel permeation chromatography.

25. An Apolipoprotein A-I (“ApoA-I”) formulation comprising ApoA-I and one or more lipids, wherein the ApoA-I and the lipids are in the form of lipoprotein complexes, for use in treating a subject having one or more symptoms associated with leukocytosis.

26. An Apolipoprotein A-I (“ApoA-I”) formulation comprising ApoA-I and one or more lipids, wherein the ApoA-I and the lipids are in the form of lipoprotein complexes, for use in treating a subject having endothelial dysfunction.

27. An Apolipoprotein A-I (“ApoA-I”) formulation comprising ApoA-I and one or more lipids, wherein the ApoA-I and the lipids are in the form of lipoprotein complexes, for use in treating a subject having or at risk of developing carditis.

28. An Apolipoprotein A-I (“ApoA-I”) formulation comprising ApoA-I and one or more lipids, wherein the ApoA-I and the lipids are in the form of lipoprotein complexes, for use in treating a subject having or at risk of developing leukocytosis.

29. An Apolipoprotein A-I (“ApoA-I”) formulation comprising ApoA-I and one or more lipids, wherein the ApoA-I and the lipids are in the form of lipoprotein complexes, for use in treating a subject experiencing acute coronary syndrome or stroke or who has experienced acute coronary syndrome or stroke.

30. The formulation for use according to any one of embodiments 25 to 29, wherein the ApoA-I has the amino acid sequence of amino acids 25-267 of SEQ ID NO:2.

31. The formulation for use according to any one of embodiments 25 to 30, wherein the ApoA-I is recombinantly expressed.

32. The formulation for use according to any one of embodiments 25 to 31, wherein said one or more lipids comprise neutral lipids.

33. The formulation for use according to embodiment 32, wherein the neutral lipids comprise sphingomyelin.

34. The formulation for use according to embodiment 33, wherein the neutral lipids consist of sphingomyelin.

35. The formulation for use according to embodiment 33 or embodiment 34, wherein the sphingomyelin comprises natural sphingomyelin.

36. The formulation for use according to embodiment 35, wherein the natural sphingomyelin is chicken egg sphingomyelin.

37. The formulation for use according to embodiment 33 or embodiment 34, wherein the sphingomyelin comprises synthetic sphingomyelin.

38. The formulation for use according to embodiment 37, wherein the synthetic sphingomyelin is palmitoylsphingomyelin.

39. The formulation for use according to any one of embodiments 25 to 34, wherein the one or more lipids further comprise negatively charged lipids.

40. The formulation for use according to embodiment 39, wherein the negatively charged lipids comprise 1,2-dipalmitoyl-sn-glycero-3-[phospho-rac-(1-glycerol)](“DPPG”) or a salt thereof.

41. The formulation for use according to embodiment 40, wherein the negatively charged lipids consist of DPPG or a salt thereof.

42. The formulation for use according to any one of embodiments 35 to 41, wherein the molar ratio of the components of the negatively charged lipid to the neutral lipid to the ApoA-I in the formulation is 2-6:90-120:1.

43. The formulation for use according to any one of embodiments 32 to 42, wherein said lipids consist of 95 to 99 weight % neutral phospholipid and 1 to 5 weight % negatively charged phospholipid.

44. The method embodiment 43, wherein said lipids consist of 96 to 98 weight % neutral phospholipid and 2 to 4 weight % negatively charged phospholipid.

45. The formulation for use according to embodiment 44, wherein said lipids consists of 97 weight % neutral phospholipid and 3 weight % negatively charged phospholipid.

46. The formulation for use according to any one of embodiments 25 to 45 which has an ApoA-I to lipid ratio ranging from 1:2 to 1:3 by weight.

47. The formulation for use according to embodiment 46, which has an ApoA-I to lipid ratio of about 1:2.7 by weight.

48. The formulation for use according to any one of embodiments 25 to 47, wherein the lipoprotein complexes are at least 95% homogeneous as reflected by a single peak in gel permeation chromatography.

49. An Apolipoprotein A-I (“ApoA-I”) formulation comprising ApoA-I and one or more lipids, wherein the ApoA-I and the lipids are in the form of lipoprotein complexes, for use in the manufacture of a medicament for one or more symptoms associated with leukocytosis.

50. An Apolipoprotein A-I (“ApoA-I”) formulation comprising ApoA-I and one or more lipids, wherein the ApoA-I and the lipids are in the form of lipoprotein complexes, for use in the manufacture of a medicament for endothelial dysfunction.

51. An Apolipoprotein A-I (“ApoA-I”) formulation comprising ApoA-I and one or more lipids, wherein the ApoA-I and the lipids are in the form of lipoprotein complexes, for use in the manufacture of a medicament for carditis or a risk of developing carditis.

52. An Apolipoprotein A-I (“ApoA-I”) formulation comprising ApoA-I and one or more lipids, wherein the ApoA-I and the lipids are in the form of lipoprotein complexes, for use in the manufacture of a medicament for leukocytosis or a risk of developing leukocytosis.

53. An Apolipoprotein A-I (“ApoA-I”) formulation comprising ApoA-I and one or more lipids, wherein the ApoA-I and the lipids are in the form of lipoprotein complexes, for use in the manufacture of a medicament for acute coronary syndrome or stroke.

54. The formulation for use according to any one of embodiments 49 to 53, wherein the ApoA-I has the amino acid sequence of amino acids 25-267 of SEQ ID NO:2.

55. The formulation for use according to any one of embodiments 49 to 54, wherein the ApoA-I is recombinantly expressed.

56. The formulation for use according to any one of embodiments 49 to 55, wherein said one or more lipids comprise neutral lipids.

57. The formulation for use according to embodiment 56, wherein the neutral lipids comprise sphingomyelin.

58. The formulation for use according to embodiment 57, wherein the neutral lipids consist of sphingomyelin.

59. The formulation for use according to embodiment 57 or embodiment 58, wherein the sphingomyelin comprises natural sphingomyelin.

60. The formulation for use according to embodiment 59, wherein the natural sphingomyelin is chicken egg sphingomyelin.

61. The formulation for use according to embodiment 57 or embodiment 58, wherein the sphingomyelin comprises synthetic sphingomyelin.

62. The formulation for use according to embodiment 61, wherein the synthetic sphingomyelin is palmitoylsphingomyelin.

63. The formulation for use according to any one of embodiments 49 to 58, wherein the one or more lipids further comprise negatively charged lipids.

64. The formulation for use according to embodiment 63, wherein the negatively charged lipids comprise 1,2-dipalmitoyl-sn-glycero-3-[phospho-rac-(1-glycerol)](“DPPG”) or a salt thereof.

65. The formulation for use according to embodiment 64, wherein the negatively charged lipids consist of DPPG or a salt thereof.

66. The formulation for use according to any one of embodiments 59 to 65, wherein the molar ratio of the components of the negatively charged lipid to the neutral lipid to the ApoA-I in the formulation is 2-6:90-120:1.

67. The formulation for use according to any one of embodiments 56 to 66, wherein said lipids consist of 95 to 99 weight % neutral phospholipid and 1 to 5 weight % negatively charged phospholipid.

68. The method embodiment 67, wherein said lipids consist of 96 to 98 weight % neutral phospholipid and 2 to 4 weight % negatively charged phospholipid.

69. The formulation for use according to embodiment 68, wherein said lipids consists of 97 weight % neutral phospholipid and 3 weight % negatively charged phospholipid.

70. The formulation for use according to any one of embodiments 49 to 69 which has an ApoA-I to lipid ratio ranging from 1:2 to 1:3 by weight.

71. The formulation for use according to embodiment 70, which has an ApoA-I to lipid ratio of about 1:2.7 by weight.

72. The formulation for use according to any one of embodiments 49 to 71, wherein the lipoprotein complexes are at least 95% homogeneous as reflected by a single peak in gel permeation chromatography.

73. The method or formulation for use according to any one of embodiments 1 to 72, wherein the dose is administered twice per day.

74. The method or formulation for use according to any one of embodiments 1 to 73, wherein the dose is administered for five days.

75. The method or formulation for use according to any one of embodiments 1 to 74, wherein the dose is effective to reduce the subject's white blood cell count by at least 2000 WBCs per microliter within five days of the first administration of the lipid binding protein-based complex.

76. The method or formulation for use according to any one of embodiments 1 to 75, wherein the dose is effective to reduce the subject's white blood cell count by at least 3000 WBCs per microliter within five days of the first administration of the lipid binding protein-based complex.

What is claimed is:

1. A method of treating a subject having one or more symptoms associated with leukocytosis, comprising administering to the subject a dose of a lipid binding protein-based complex effective to reduce the subject's white blood cell count and/or ameliorate one or more of the one or more symptoms associated with leukocytosis.

2. The method of claim 1, wherein the one or more symptoms comprise a fever; bleeding or bruising; sweating; pain or tingling in the legs, arms, or abdomen; one or more vision problems; unclear thinking; loss of appetite; and/or trouble breathing (e.g., shortness of breath, below normal blood oxygen levels).

3. The method of claim 1 or claim 2, wherein the dose of a lipid binding protein-based complex is effective to ameliorate one or more of the one or more symptoms associated with leukocytosis.

4. The method of any one of claims 1 to 3, wherein the subject has or is at risk of developing leukocytosis due to inflammation, an infection, a white blood cell disorder, physical stress, emotional stress, medication, or an allergic reaction.

5. The method of any one of claims 1 to 4, wherein the subject has an infection.

6. The method of claim 5, wherein the infection is a viral infection, optionally wherein the viral infection is a coronavirus infection, which is optionally COVID-19; a bacterial infection; a fungal infection; or a parasitic infection.

7. The method of any one of claims 1 to 6, wherein the subject has diabetes.

8. The method of any one of claims 1 to 7, wherein the subject has a white blood cell disorder.

9. The method of any one of claims 1 to 8, wherein the subject has leukocytosis.

10. The method of claim 9, wherein the subject has a white blood cell count above 11×10⁹ per L.

11. The method of any one of claims 1 to 10, wherein the subject is at risk of leukocytosis.

12. A method of treating a subject having endothelial dysfunction, comprising administering to the subject a dose of a lipid binding protein-based complex, wherein the dose is a high dose.

13. The method of claim 12, wherein the dose is effective to improve the subject's vascular endothelial function, optionally wherein vascular endothelial function is measured by circulating VCAM-1 and/or ICAM-1.

14. The method of claim 13, wherein the dose is effective to reduce the subject's circulating VCAM-1 by at least 100 ng/mL within two days of the first administration of the lipid binding protein-based complex.

15. The method of claim 13 or claim 14, wherein the dose is effective to reduce the subject's circulating VCAM-1 by up to 400 ng/mL within five days of the first administration of the lipid binding protein-based complex.

16. The method of any one of claims 13 to 15, wherein the dose is effective to reduce the subject's circulating ICAM-1 by at least 50 ng/mL within two days of the first administration of the lipid binding protein-based complex.

17. The method of any one of claims 13 to 16, wherein the dose is effective to reduce the subject's circulating ICAM-1 by up to 125 ng/mL within five days of the first administration of the lipid binding protein-based complex.

18. A method of treating a subject having or at risk of developing carditis, comprising administering to the subject a dose of a lipid binding protein-based complex, optionally wherein the carditis is myocarditis and/or pericarditis.

19. The method of claim 18, wherein the subject has an infection, such as a viral infection, which is optionally a coronavirus infection, which is optionally COVID-19; or a bacterial infection.

20. The method of claim 18 or claim 19, wherein the dose of the lipid binding protein-based complex is effective to ameliorate one or more symptoms of the carditis.

21. The method of any one of claims 18 to 20, wherein the subject is at risk of carditis.

22. The method of claim 21, wherein the dose of the lipid binding protein-based complex is effective to prevent the carditis or reduce the severity of the carditis.

23. The method of any one of claims 1 to 22, wherein the dose is effective to reduce the subject's circulating VCAM-1 and/or ICAM-1.

24. A method of treating a subject having or at risk of developing leukocytosis, comprising administering to the subject a dose of a lipid binding protein-based complex effective to reduce the subject's white blood cell count.

25. A method of treating a subject experiencing acute coronary syndrome or stroke or who has experienced acute coronary syndrome or stroke, comprising administering to the subject a dose of a lipid binding protein-based complex, wherein the dose is a high dose.

26. The method of claim 25, wherein the subject is experiencing or has experienced acute coronary syndrome, optionally wherein the acute coronary syndrome is myocardial infarction (for example ST-elevation myocardial infarction or non-ST elevation myocardial infarction) or unstable angina.

27. The method of claim 25, wherein the subject is experiencing or has experienced a stroke.

28. A method of treating a subject having one or more symptoms associated with leukocytosis, comprising administering to the subject a dose of an Apolipoprotein A-I (“ApoA-I”) formulation comprising ApoA-I and one or more lipids, wherein the ApoA-I and the lipids are in the form of lipoprotein complexes, effective to reduce the subject's white blood cell count and/or ameliorate one or more of the one or more symptoms associated with leukocytosis.

29. A method of treating a subject having endothelial dysfunction, comprising administering to the subject an ApoA-I formulation comprising ApoA-I and one or more lipids, wherein the ApoA-I and the lipids are in the form of lipoprotein complexes.

30. A method of treating a subject having or at risk of developing carditis, comprising administering to the subject an ApoA-I formulation comprising ApoA-I and one or more lipids, wherein the ApoA-I and the lipids are in the form of lipoprotein complexes.

31. A method of treating a subject having or at risk of developing leukocytosis, comprising administering to the subject a dose of an ApoA-I formulation comprising ApoA-I and one or more lipids, wherein the ApoA-I and the lipids are in the form of lipoprotein complexes, effective to reduce the subject's white blood cell count.

32. A method of treating a subject experiencing acute coronary syndrome or stroke or who has experienced acute coronary syndrome or stroke, comprising administering to the subject an ApoA-I formulation comprising ApoA-I and one or more lipids, wherein the ApoA-I and the lipids are in the form of lipoprotein complexes.

33. The method of any one of claims 28 to 32, wherein the ApoA-I has the amino acid sequence of amino acids 25-267 of SEQ ID NO:2.

34. The method of any one of claims 28 to 33, wherein the ApoA-I is recombinantly expressed.

35. The method of any one of claims 28 to 34, wherein said one or more lipids comprise neutral lipids.

36. The method of claim 35, wherein the neutral lipids comprise sphingomyelin.

37. The method of claim 36, wherein the neutral lipids consist of sphingomyelin.

38. The method of claim 36 or claim 37, wherein the sphingomyelin comprises natural sphingomyelin.

39. The method of claim 38, wherein the natural sphingomyelin is chicken egg sphingomyelin.

40. The method of claim 36 or claim 37, wherein the sphingomyelin comprises synthetic sphingomyelin.

41. The method of claim 40, wherein the synthetic sphingomyelin is palmitoylsphingomyelin.

42. The method of any one of claims 28 to 37, wherein the one or more lipids further comprise negatively charged lipids.

43. The method of claim 42, wherein the negatively charged lipids comprise 1,2-dipalmitoyl-sn-glycero-3-[phospho-rac-(1-glycerol)](“DPPG”) or a salt thereof.

44. The method of claim 43, wherein the negatively charged lipids consist of DPPG or a salt thereof.

45. The method of any one of claims 38 to 44, wherein the molar ratio of the components of the negatively charged lipid to the neutral lipid to the ApoA-I in the formulation is 2-6:90-120:1.

46. The method of any one of claims 35 to 45, wherein said lipids consist of 95 to 99 weight % neutral phospholipid and 1 to 5 weight % negatively charged phospholipid.

47. The method claim 46, wherein said lipids consist of 96 to 98 weight % neutral phospholipid and 2 to 4 weight % negatively charged phospholipid.

48. The method of claim 47, wherein said lipids consists of 97 weight % neutral phospholipid and 3 weight % negatively charged phospholipid.

49. The method of any one of claims 28 to 48 which has an ApoA-I to lipid ratio ranging from 1:2 to 1:3 by weight.

50. The method of claim 49, which has an ApoA-I to lipid ratio of about 1:2.7 by weight.

51. The method of any one of claims 28 to 50, wherein the lipoprotein complexes are at least 95% homogeneous as reflected by a single peak in gel permeation chromatography.

52. The method of any one of claims 1 to 51, wherein the dose is administered twice per day.

53. The method of any one of claims 1 to 52, wherein the dose is administered for five days.

54. The method of any one of claims 1 to 53, wherein the dose is effective to reduce the subject's white blood cell count by at least 2000 WBCs per microliter within five days of the first administration of the lipid binding protein-based complex.

55. The method of any one of claims 1 to 54, wherein the dose is effective to reduce the subject's white blood cell count by at least 3000 WBCs per microliter within five days of the first administration of the lipid binding protein-based complex.


SEQUENCE LISTING (2 sequences)

SEQ ID NO: 1 - protein, 190 aa, synthetic construct
PPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEY

SEQ ID NO: 2 - protein, 267 aa, synthetic construct
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ